(19) United States
(12) Patent Application Publication
(10) Pub. No.: US 2011/0262407 A1
(43) Pub. Date: Oct. 27, 2011

(54) TREATMENT WITH ALPHA7 SELECTIVE LIGANDS

(75) Inventors: Merouane Bencherif, Winston-Salem, NC (US); Kristen Jordan, Clemmons, NC (US); Terry Hauser, Winston-Salem, NC (US); Steven M. Toler, Winston-Salem, NC (US); Sharon Rae Letchworth, Kernersville, NC (US); David C. Kombo, Winston-Salem, NC (US)

(73) Assignee: Targacept, Inc., Winston-Salem, NC (US)

(21) Appl. No.: 13/124,829

(22) PCT Filed: Nov. 9, 2009
(86) PCT No.: PCT/US0209/063727

§ 371 Date: Jun. 21, 2011

Related U.S. Application Data
(60) Provisional application No. 61/113,282, filed on Nov. 11, 2008.
(60) Provisional application No. 61/179,136, filed on May 18, 2009.

(51) Int. Cl. A61K 35/12; A61P 37/06; A61P 35/00; A61P 9/00; A61P 17/02; A61P 27/16; A61P 27/02; A61P 3/10; A61P 19/00; A61K 31/439; A61P 17/14

(52) U.S. Cl. 424/93.7; 514/305

(57) ABSTRACT

The present invention includes methods, uses, and selective alpha7 nAChR ligands for treating or preventing disease and disorders in which stimulation of neurogenesis is ameliorative; namely, wherein the recruitment of neurogenesis is therapeutic.

### FIELD OF THE INVENTION

The present invention includes methods, uses, and selective α7 nAChR ligands for treating or preventing disease and disorders in which stimulation of neurogenesis is ameliorative, namely, the recruitment of neurogenesis is therapeutic.

### BACKGROUND

Neurogenesis is the process by which new nerve cells are generated. In neurogenesis, there is active production of new neurons, astrocytes, glia and other neural lineages from undifferentiated neural progenitor or stem cells. Until recently, neurogenesis in mammals was believed to occur only during the embryonic and early post-natal periods and do not play a significant role in the adult nervous system. However, it is now accepted that neurogenesis occurs in at least two brain regions in adult mammals, the hippocampus and olfactory bulb (Ehninger and Kempermann, Cell Tissue Res 331: 243-50 (2008)). In both regions new neurons arise from endogenous progenitor cells that are viable throughout adult life. Hippocampal neurogenesis is required for some types of hippocampal-dependent learning (Bruel-Jungerman et al., Rev Neurosci 18: 93-114 (2007)). Recently the relevance of hippocampal neurogenesis to the pathophysiology and treatment of mood disorders has received much attention. It is now known that all major pharmacological and non-pharmacological treatments for depression increase hippocampal neurogenesis (Malber et al., J Neurosci 20: 9104-9110 (2000); Santarelli et al., Science 301: 805-809 (2003)). Conversely, suppression of hippocampal neurogenesis in rodents blocks behavioral responses in some antidepressant-sensitive tests (Santarelli et al., Science 301: 805-809 (2003)). Altered hippocampal neurogenesis may also play a pathophysiological role in neurodegenerative disorders such as Alzheimer's disease (Abdipranoto et al., CNS Neurol Disord Drug Targets 7: 187-210 (2008)). It is not clear how much neurogenesis occurs normally in other brain regions. However, neural progenitors are found throughout the brain and nervous system, including both neurogenic and non-neurogenic regions. The existence of endogenous neural progenitors even in non-neurogenic brain regions suggests that the potential of these cells may be unlocked to repair cellular injuries resulting from stroke, trauma, neurodegenerative diseases, radiation and chemotherapy-induced damage and many other neural insults.

It has been shown that various neurotransmitter systems in the brain can regulate or trigger the processes involved in neurogenesis. Specifically, cholinergic systems in the brain appear to figure prominently in regulating neurogenesis (Kotani et al., Neuroscience 142: 505-14 (2006)). Data showing that lesions of the forebrain cholinergic projections in the brain inhibit neurogenesis (Cooper-Kuhn et al., J Neurosci Res 77: 155-65 (2004)) supports the role of the cholinergic system in promoting survival of neuronal progenitors and immature neurons within regions of adult neurogenesis. There is evidence that pharmacological manipulation of the cholinergic system (e.g., with cholinesterase inhibitors) can modulate adult hippocampal neurogenesis. For example, activation of the cholinergic system promotes survival of newborn neurons in the adult hippocampal dentate gyrus and olfactory bulb under both normal and stressed conditions (Kaneko et al., Genes Cells 11: 1145-59 (2006)). The hippocampus appears to be a focal point for cholinergic control of neurogenic processes. For example, hippocampus-mediated learning enhances neurogenesis in the adult dentate gyrus, and this process has been suggested to be involved in memory formation (Bruel-Jungerman et al., Rev Neurosci 18: 93-114 (2007)). The hippocampus receives abundant cholinergic innervation and acetylcholine (ACh) plays an important role in learning and Alzheimer's disease (AD) pathophysiology. Impaired cholinergic function in AD may in part contribute to deficits in learning and memory through reductions in the formation of new hippocampal neurons.

Despite the implication of acetylcholine and cholinergic systems in neurogenesis, the specific acetylcholine receptor target(s) involved have not been characterized. One of the primary cholinergic receptor systems that regulate neurotransmitter release in the CNS is the family of ligand-gated ion channel receptors, nicotinic acetylcholine receptors (nAChRs). There is some indication that nAChRs may contribute to neurogenesis in that nicotine has been shown to significantly enhance neuronal precursor cell proliferation in the subventricular zone of the brain (from which cells can migrate to the olfactory bulb) of adult rat brain, and pre-treatment with mecamylamine, a nonselective nAChR antagonist, blocks the enhanced precursor proliferation by nicotine (Mudo et al., Neuroscience 145: 470-83 (2007)). Although, some studies have shown that nicotine blocks neurogenic activity in the brain (Shingo and Kito, J Neural Transm 112: 1475-8 (2005)), because nicotine is a relatively non-selective nAChR agonist the involvement of specific nAChR subtypes, if any, cannot be inferred from such studies.

Based on data presented herein, we identify the alpha7 nAChR subtype as a potential target for therapeutic intervention in conditions that require activation of neurogenesis and demonstrate that alpha7-selective compounds will stimulate neurogenic activity in the brain. Previous work on the pattern of development of the alpha7 receptor suggests that it may influence processes as diverse as cell migration, dendritic elaboration and apoptosis during hippocampal development and maturation (Adams et al., Brain Res Dev Brain Res 139: 175-87 (2002)). The modulatory role of alpha7 nAChRs also extends to processes involved in neuroprotection, inhibition of apoptosis and anti-inflammation, all of which could potentially influence the process of neurogenesis either directly or indirectly (Suzuki et al., J Neurosci Res 83: 1461-70 (2006)). Studies have shown that these processes can be activated by the endogenous neurotransmitter acetylcholine since treatment with acetylcholinesterase inhibitors can attenuate inflammation (Nizri et al., Drug News Perspect. 20: 421-9 (2007)), presumably by increasing the acetylcholine (ACh) concentration near immune cells and making it available for interaction with alpha7 nAChRs expressed on these cells. Exogenously administered nAChR agonists can exert similar effects. For example, the prototypical nAChR agonist nicotine has been found to inhibit death of PC12 cells in vitro (Yamashita et al., Neurosci Lett 213: 145-7 (1996)). In rat primary cultured microglia, nicotine enhances P2X(7) receptor-mediated tumor necrosis factor (TNF) release and suppresses lipopolysaccharide (LPS)-induced TNF release (Suzuki et al., 2006). These effects are thought to be mediated by alpha7 nAChRs and involve modification of microglia activation towards a neuroprotective role by suppressing the inflammatory state and strengthening the protective function. The selective alpha7 receptor agonists, 3-[2,4-dimethoxybenzylidene]anabaseine (DMXB) (deFiebre and deFiebre, Alcohol 31: 149-53 (2003)), and TC-1698 (Marrero et al., J Pharmacol Exp Ther 309: 16-27 (2004)) have been reported to exert cytoprotective effects. It was also recently shown that nicotine activates the growth promoting enzyme janus kinase 2 (JAK2) in PC12 cells, and that pre-incubation of these cells with the JAK2 specific inhibitor AG-490 blocks the nicotine-induced activation of neuroprotective signaling cascades (Shaw et al., J Biol Chem 277: 44920-4 (2002)).

The need for novel compounds that can inhibit inflammatory cascades, promote neurogenesis, or both is emphasized by the lack of effective therapies addressing the chronic insidious inflammation and subsequent neuronal degeneration following a number of brain insults including but not restricted to micro-infarcts, inflammation, infection, trauma, chemotherapy, radiation therapy, and neurodegenerative processes. In this regard, compounds selective for alpha7 nAChRs have been shown previously to ameliorate neuronal death associated with growth factor deprivation, 1-methyl-4-phenyl-1,2,3,6-tetrahydropyridine (MPTP) exposure, glutamate, or beta-amyloid-induced neuronal death.

Background reference may be made to one or more of: Abdipranoto A, Wu S, Stayte S, Vissel B, The role of neurogenesis in neurodegenerative diseases and its implications for therapeutic development CNS Neurol Disord Drug Targets 7:187-210 (2008); Adams C E, Broide R S, Chen Y, Winzer-Serhan U H, Henderson T A, Leslie F M, Freedman R., Development of the alpha7 nicotinic cholinergic receptor in rat hippocampal formation, Brain Res Dev Brain Res 139: 175-87 (2002); Bruel-Jungerman E, Rampon C, Laroche S, Adult hippocampal neurogenesis, synaptic plasticity and memory: facts and hypothesis, Rev Neurosci 18: 93-114 (2007); Caldarone B J, Harrist A, Cleary M A, Beech R D, King S L, Picciotto M R, High-affinity nicotinic acetylcholine receptors are required for antidepressant effects of amitriptyline on behavior and hippocampal cell proliferation, Biological Psychiatry 5: 657-664 (2004); Cooper-Kuhn C M, Winkler J, Kuhn H G, Decreased neurogenesis after cholinergic forebrain lesion in the adult rat, J Neurosci Res 77: 155-65 (2004); de Fiebre N C, de Fiebre C M, Alpha 7 nicotinic acetylcholine receptor-mediated protection against ethanol-induced neurotoxicity, Alcohol 31: 149-53 (2003); Dowling O, Rochelson B, Way K, Al-Abed Y, Metz C N, Nicotine inhibits cytokine production by placenta cells via NF-kappaB: potential role in pregnancy-induced hypertension, Mol Med 13: 576-83 (2007); Ehninger D, Kempermann G, Neurogenesis in the adult hippocampus, Cell Tissue Res 331: 243-50 (2008); Elder G A, Gama Sosa M A, Research update: neurogenesis in adult brain and neuropsychiatric disorders, Mt Sinai J Med 73: 931-40 (2006); Gatto G J, Bohme G A, Caldwell W S, Letchworth S R, Traina V M, Obinu M C, Laville M, Reibaud M, Pradier L, Dunbar G, Bencherif M, TC-1734: An orally active neuronal nicotinic acetylcholine receptor modulator with antidepressant, neuroprotective and long-lasting cognitive effects. CNS Drug Rev 10:147-66 (2004); Harrist A, Beech R D, King S L, Zanardi A, Cleary M A, Caldarone B J, Eisch A, Zoli M, Picciotto M R, Alteration of hippocampal cell proliferation in mice lacking the beta 2 subunit of the neuronal nicotinic acetylcholine receptor, Synapse 54: 200-6 (2004); Kaneko N, Okano H, Sawamoto K, Role of the cholinergic system in regulating survival of newborn neurons in the adult mouse dentate gyrus and olfactory bulb, Genes Cells 11: 1145-59 (2006); Kotani S, Yamauchi T, Teramoto T, Ogura H, Pharmacological evidence of cholinergic involvement in adult hippocampal neurogenesis in rats, Neuroscience 142: 505-14 (2006); Malberg J E, Eisch A J, Nestler E J, Duman R S, Chronic antidepressant treatment increases neurogenesis in adult rat hippocampus, J Neurosci 20:9104-9110 (2000); Marrero M B, Papke R L, Bhatti B S, Shaw S, Bencherif M, The neuroprotective effect of 2-(3-pyridyl)-1-azabicyclo[3.2.2]nonane (TC-1698), a novel alpha7 ligand, is prevented through angiotensin II activation of a tyrosine phosphatase, J Pharmacol Exp Ther 309: 16-27 (2004); Mohapel P, Leanza G, Kokaia M, Lindvall O, Forebrain acetylcholine regulates adult hippocampal neurogenesis and learning, Neurobiol Aging 26: 939-46 (2005); Mode) G, Belluardo N, Mauro A, Fuxe K, Acute intermittent nicotine treatment induces fibroblast growth factor-2 in the subventricular zone of the adult rat brain and enhances neuronal precursor cell proliferation, Neuroscience 145: 470-83 (2007); Nizri E, Wirguin I, Brenner T, The role of cholinergic balance perturbation in neurological diseases, Drug News Perspect. 20: 421-9 (2007); Perera T D, Park S, Nemirovskaya Y, Cognitive role of neurogenesis in depression and antidepressant treatment, Neuroscientist 14: 326-38 (2008); Picciotto M R, Brunzell D H, Caldarone B J, Effect of nicotine and nicotinic receptors on anxiety and depression, Neuroreport 13: 1097-1106 (2002); Santarelli L, Saxe M, Gross C, Surget A, Battaglia F, Dulawa S, et al, Requirement of hippocampal neurogenesis for the behavioral effects of antidepressants, Science 301: 805-809 (2003); Shankaran M, King C, Lee J, Busch R, Wolff M, Hellerstein M K, Discovery of novel hippocampal neurogenic agents by using an in vivo stable isotope labeling technique, J Pharmacol Exp Ther 319:1172-1182 (2006); Shankaran M, Marino M E, Busch R, Keim C, King C, Lee J, Killion S, Awada M, Hellerstein M K, Measurement of brain microglial proliferation rates in vivo in response to neuroinflammatory stimuli: Application to drug discovery, J Neurosci Res 85: 2374-84 (2007); Shaw S, Bencherif M, Marrero M B, Janus kinase 2, an early target of alpha 7 nicotinic acetylcholine receptor-mediated neuroprotection against Abeta-(1-42) amyloid, J Biol Chem 277: 44920-4 (2002); Shytle R D, Silver A A, Lukas R J, Newman M B, Sheehan D V, Sanberg P R, Nicotinic acetylcholine receptors as targets for antidepressants, Mol Psychiatry 7: 525-535 (2002); Shingo A S, Kito S, Effects of nicotine on neurogenesis and plasticity of hippocampal neurons, J Neural Transm 112: 1475-8 (2005); Suzuki T, Hide I, Matsubara A, Hama C, Harada K, Miyano K, Andrä M, Matsubayashi H, Sakai N, Kohsaka S, Inoue K, Nakata Y, Microglial alpha7 nicotinic acetylcholine receptors drive a phospholipase C/IP3 pathway and modulate the cell activation toward a neuroprotective role Neurosci Res 83:1461-70 (2006); Wang N, Orr-Urtreger A, Korczyn A D, The role of neuronal nicotinic acetylcholine receptor subunits in autonomic ganglia: lessons from knockout mice, Prog Neurobiol. 68: 341-60 (2002); Yamashita H, Nakamura S, Nicotine rescues PC12 cells from death induced by nerve growth factor deprivation. Neurosci Lett 213:145-7 (1996); Zipp F, Aktas O, The brain as a target of inflammation: common pathways link inflammatory and neurodegenerative diseases, Trends Neurosci 29: 518-27 (2006); Lewy Body-Like Pathology in Long-Term Embryonic Nigral Transplants in Parkinson's Disease, Nature Medicine 14, 504-506 (1 May 2008)—including citation thereof in NeuroInvestment, January 2009; each of which is incorporated by reference with regard to the background teaching of nAChR modulators.

### SUMMARY OF THE INVENTION

Presented herein is evidence for the direct involvement of alpha7 nAChRs in neurogenesis. Specifically, compounds that selectively activate alpha7 nAChRs demonstrate neurogenesis in vivo using hippocampal progenitor cell proliferation models. These new findings implicate alpha7 nAChRs as modulators of neurogenesis and establish their potential as therapeutic targets for treating diseases and disorders in which stimulation of neurogenesis is ameliorative. Further, there may be an added benefit of alpha7-selective compounds through anti-inflammatory processes mediated by (nuclear factor-kappa B) NFκB and pro-inflammatory pathways (Dowling et al., Mol Med 13: 576-83 (2007)). Potential synergy between the neurogenesis and anti-inflammatory properties of alpha7-selective compounds in the treatment of disease and disorders makes them even more attractive as therapeutic agents. Additionally, as is demonstrated herein, an alpha 7 agonist is believed to provide viability for cell therapy. An alpha 7 agonist may be used in conjunction with stem cell implants for underlying neuroprotection and/or disease modification in order for the implanted cells to remain healthy and become functional.

Because the beta2-containing nAChR subtypes have also been implicated in processes related to cell survival (Harrist et al., Synapse 54: 200-6 (2004)), the potential also exists for achieving additional efficacy with compounds that target both alpha4beta2 and alpha7 pharmacology. The combined effects on neurogenesis and inflammation will provide the potential to minimize deterioration and/or ameliorate symptoms of patients in a number of CNS (related) disease states or conditions, including but not limited to adrenoleukodystrophy (ALD), multiple sclerosis (MS), stroke, Parkinson's disease (PD), ischemia-reperfusion injury (due to peripheral insult), meningitis, autoimmune disease, Alzheimer's disease, brain trauma and injury, radiation-induced cognitive deficits, chemotherapy-induced cognitive deficits, depression and Huntington's disease (HD).

Irradiation of primary and metastatic brain cancer can lead to devastating structural and functional deficits, including vasculopathy, demyelination, gliosis, white matter necrosis and chronic cognitive impairment several months to years after irradiation. Currently, no successful treatments or effective preventive strategies exist to overcome these deficits. It has been suggested that radiation-induced cognitive impairment is due, in part, to acute and chronic inflammation within the brain. Activation of alpha7 nAChRs can improve cognitive performance in rats, rabbits, and monkeys, whereas blockade of those receptors impairs performance. Recent studies indicate that activation of alpha7 nAChRs using agonists prevents the translocation of NF-κB to the nucleus and activates the JAK/signal transducer and activator of transcription STAT-3 pathway, reducing the release of pro-inflammatory cytokines. We now disclose that treating rat brain microvascular endothelial cells using alpha7-selective agonists prevents radiation-induced inflammatory responses.

Of the estimated 17,000 primary brain tumors diagnosed in the United States each year, approximately 60% are gliomas. Glioblastoma multiforme (GBM) is by far the most common and most malignant of the glial tumors and will be used herein as a general term to describe the class of tumors. No significant advancements in the treatment of glioblastoma have occurred in the past 25 years. Without therapy, patients with GBMs uniformly die within 3 months. Patients treated with optimal therapy have a median survival time of approximately 12 months.

Currently, first line therapy for GBM includes surgical ablation, directed radiotherapy, and temozolamide. Targeted radiotherapy produces reactive oxygen species (superoxide ion, hydroxyl radical, hydrogen peroxide) which are believed to be responsible for its cytotoxic effects. Cells that can adapt to an environment of elevated levels of reactive oxygen species through up-regulation of oxidative stress mediation mechanisms can curtail the effects of these reactive oxygen species and improve their chance of survival. For example, the T98G cell line which is resistant to the effects of ionizing radiation has been observed to have 14 times the glutathione concentrations of NB9 cells (which are sensitive to ionizing radiation). Also, U251 human glioblastoma cells exhibit induction of superoxide dismutase and glutathione peroxidase upon exposure to ionizing radiation, illustrating the adaptability of such tumor cell lines to the presence of reactive oxygen species.

In vivo and in vitro data indicate that increasing oxidative stress associated with radiotherapy may prove beneficial in the treatment of GBM. For example, two pilot studies have generated data indicating that pretreatment with pro-oxidant therapy (chloroquine) prior to radiotherapy significantly prolonged survival in patients with GBM, presumably by sensitizing resistant GBM clones to oxidative injury following radiotherapy (Sotelo et al. Annals of Internal Medicine (2006), 144(5), 337-343; Briceno et al. Surgical Neurology (2007), 67(4), 388-391. Toler et al. Neurosurg Focus. 2006 Dec. 15; 21(6):E10.)

The broad spectrum NNR antagonist mecamylamine has been reported to block the ability of nicotine, which is relatively non-selective and binds more tightly to alpha4beta2 receptors than to alpha7, to attenuate oxidative stress in a spinal cord injury model (Ravikumar et al. Molecular Brain Research (2004), 124(2), 188-198). Data presented herein demonstrate that alpha7 NNR agonists decrease the production of reactive oxygen species and ameliorate the up-regulation of pro-inflammatory cytokine (interleukin) IL-6 and intercellular adhesion molecule 1 (ICAM1) mRNA and protein in a radiation injury model, thus offering protection against radiation injury. This suggests that alpha7 receptors are primary mediators of the oxidative stress response following radiation. Therefore, alpha7 NNR antagonists may demonstrate the opposite effect and sensitize cell lines to oxidative stress induced injury and serve as a useful adjunct to directed radiotherapy of GBM. Such adjunct therapy could be accomplished in any of several fashions. For instance, the alpha7 antagonist could be administered systemically, as an adjunct, before, during, or after radiation therapy. Alternatively, an alpha7 NNR antagonists could be applied locally, at the site of tumor excision, during or immediately following surgical ablation. Finally, since alpha7 NNR agonists may protect against radiation injury in healthy areas of the brain, it is conceivable that a combination therapy, in which one administers an alpha7 NNR antagonist locally (to enhance the effectiveness of the radiotherapy) and an alpha7 NNR agonist systemically (to protect healthy tissue) before or during radiotherapy, may be very effective.

One aspect of the present invention includes a method for treating or preventing disorders or conditions susceptible to recruitment of neurogenesis comprising administering a selective alpha7 agonist.

Another aspect of the present invention includes a method for providing neuroprotection comprising administering a selective alpha7 agonist.

Another aspect of the present invention includes inhibiting progression of a central nervous system disorder comprising administering a selective alpha7 agonist.

In one embodiment of these aspects, the alpha7 agonist increases the proliferation of progenitor cells in the hippocampus. In another embodiment, the disorder or condition is selected from learning and memory disorders, epilepsy, psychiatric disorders, depression, bipolar disorder, post traumatic stress disorder, neurodegenerative diseases, Alzheimer's disease, Parkinson's disease, amyotrophic lateral sclerosis, multiple sclerosis, frontotemporal dementia, Huntington's disease, prion disease, substance abuse, addiction, dependency, head trauma, stroke, or physical injury. In one embodiment, the alpha7 agonist is used adjunctively with another therapeutic agent.

Another aspect of the present invention includes a method for treating or preventing induced cognitive deficits comprising administering an alpha7 agonist and an alpha4beta2 agonist.

In one embodiment, the administration is a single compound with dual alpha7 agonist and alpha4beta2 agonist pharmacology. In another embodiment, the induced cognitive deficit is one or more of chemotherapy-induced cognitive deficit, radiation-induced cognitive deficit, ischemia-induced cognitive deficit, autoimmune and inflammatory disease induced cognitive deficit, inflammation-induced cognitive deficit, injury-induced cognitive deficit, and neuroinflammation.

Another aspect of the present invention is a method for treating or preventing a neurobiological disorder selected from depression, major depressive disorder, addiction, physical dependence, psychological dependence, dysregulated food intake, or bipolar disorder comprising administering an alpha7 agonist and an alpha4beta2 antagonist.

In one embodiment, the administration is a single compound with dual alpha7 agonist and alpha4beta2 antagonist pharmacology.

Another aspect of the present invention is a method for treating or preventing glioblastoma multiforme comprising the administration of an alpha7 antagonist.

In one embodiment, the method is adjunctive to radiation therapy.

Another aspect of the present invention is a method for treating or preventing glioblastoma multiforme comprising administering an alpha7 agonist and an alpha7 antagonist.

In one embodiment, the alpha7 agonist is administered systemically. In another embodiment, the alpha7 antagonist is administered locally upon surgical ablation.

Another aspect of the present invention is a method for protecting stem cells against host pathology implanted in a patient comprising administration of a selective alpha 7 agonist. A further aspect includes a method for treating a CNS disorder comprising implanting one or more stem cell; and administering one or more selective alpha 7 agonist. A further aspect is a method for enhancing the survival and differentiation of stem cell implants comprising administering an alpha 7 agonist. Another aspect of the present invention is a method of inducing hippocampal neurogenesis comprising administering an alpha 7 agonist.

In one embodiment of the aforementioned aspects, the methods are useful to treat a CNS disorder. In one embodiment, the CNS disorder or condition is selected from learning and memory disorders, epilepsy, psychiatric disorders, depression, bipolar disorder, post traumatic stress disorder, neurodegenerative diseases, Alzheimer's disease, Parkinson's disease, amyotrophic lateral sclerosis, multiple sclerosis, frontotemporal dementia, Huntington's disease, prion disease, substance abuse, addiction, dependency, head trauma, stroke, or physical injury.

In another embodiment, the method treats a non-CNS disorder. In one embodiment, the disorder or condition is selected from one or more of stem-cell derived organ transplant, hematopoietic stem cell transplantation, bone marrow transplant, skin graft, cancer, neovascularization, angiogenesis, spinal cord injury, heart damage, haematopoiesis, baldness, deafness, blindness, vision impairment, birth defect, diabetes, orthopedics, and wound healing.

The present invention includes combinations of aspects and embodiments, as well as preferences, as herein described throughout the present specification.

### BRIEF DESCRIPTION OF THE FIGURES

The Figures describe results obtained according to particular embodiments of the invention and exemplify aspects of the invention but should not be construed to be limiting.

FIG. 1 is a graphic representation of the effect of anti-depressants on hippocampal progenitor proliferation in mice.

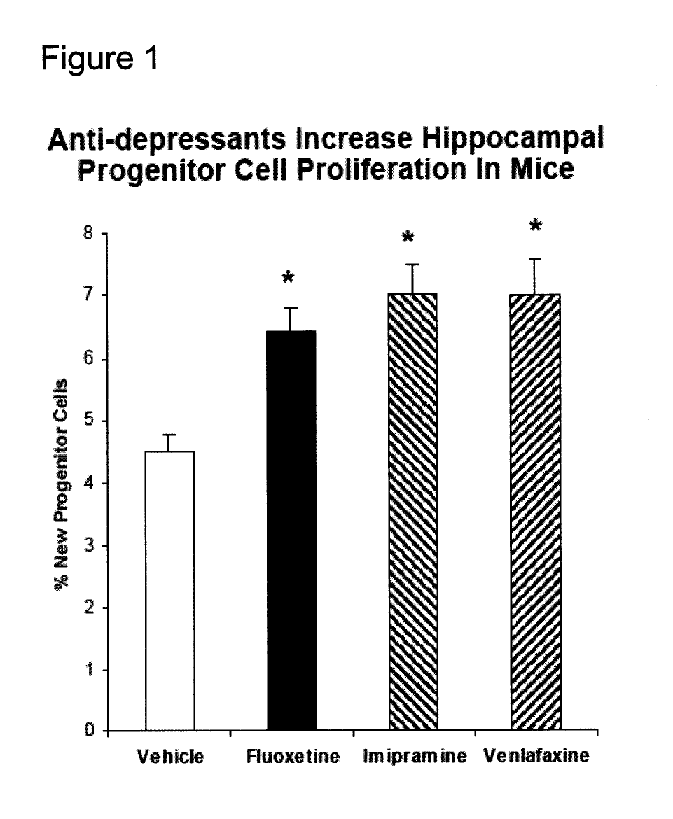

FIG. 2 is a graphic representation of the increase in progenitor cells in the hippocampus of 129SvEv mice following exposure to doses of 0.1, 0.3 or 1 mg/kg orally of the alpha7 nAChR agonist Compound A. This shows that Compound A increases neurogenesis.

FIG. 3 is a graphic representation of the effect of Compound A (1 mg/kg orally) on the incorporation of deuterium from heavy water into the DNA of microglia in c57Bl/6 mice. Based on this measure, Compound A decreased LPS-induced neuro-inflammation.

FIG. 4 shows the results of an RT-PCR analysis confirming the presence of alpha7 nAChRs in the GP 8.3 endothelial cell line.

FIG. 5 shows the dose dependent increase in levels of the pro-inflammatory cytokine IL-6 in GP 8.3 cells exposed to ionizing radiation.

FIG. 6 shows the reversal of radiation-induced increases in IL-6 in GP 8.3 cells by incubation with 10 μM Compound A.

FIG. 7 shows the reversal of radiation-induced increases in ICAM-1 in GP 8.3 cells by incubation with 10 μM Compound A.

FIG. 8 shows the reversal of radiation-induced increases in reactive oxygen species in GP 8.3 cells by incubation with 10 μM Compound A.

FIG. 9 demonstrates that protection from radiation-induced increases in ICAM-1 by Compound A can be reversed by an alpha7 nAChR antagonist, mecamylamine, confirming that the protective effects are receptor-mediated.

FIG. 10 is a graphic representation of the effects of the alpha7-selective Compound B on cell survival in PC-12 cells exposed to lethal amounts of Abeta(1-42), demonstrating that the compound is neuroprotective.

FIG. 11 is a graphic representation of the increase in progenitor cells in the hippocampus of 129SvEv mice following exposure to doses of 1 mg/kg orally of the dual pharmacology alpha7/alpha4beta2-selective nAChR agonist Compound C. This shows that Compound C increases neurogenesis.

FIG. 12 is a graphic representation of the effect of the dual pharmacology alpha7/alpha4beta2-selective nAChR agonist Compound C (0.1 mg/kg orally) on the incorporation of deuterium from heavy water into the DNA of microglia in c57Bl/6 mice. Based on this measure, Compound C decreased LPS-induced neuro-inflammation.

FIG. 13 depicts nicotine stimulation of alpha7 nAChR transduces signals to phosphatidylinositol 3-kinase and Akt via Janus kinase 2 (JAK2) in a cascade, which results in neuroprotection. Exposure to beta-amyloid results in the activation of the apoptotic enzyme caspase-3 and cleavage of the DNA-repairing enzyme poly-(ADP-ribose) polymerase. This cascade is inhibited by nicotine through JAK2 activation, and these effects are blocked by preincubation with the JAK2-specific inhibitor AG-490. Pretreatment of cells with angiotensin II blocks the nicotine-induced activation of JAK2 via the (angiotensin) AT₂ receptor and completely prevents alpha7 nAChR-mediated neuroprotective effects further suggesting a pivotal role for JAK2.

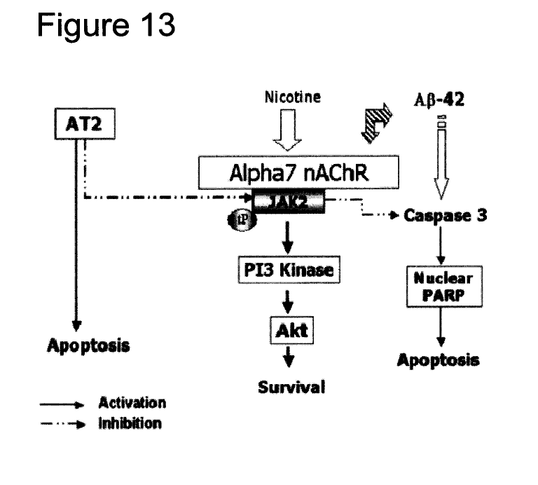

FIG. 14 is a graphic representation of the effect of Compound A on hippocampal progenitor cell proliferation, thereby demonstrating the protection of stem cells with Compound A. FIGS. 15-21 illustrate that the stem cells are functional, through a demonstration of improved cognition.

FIG. 15 demonstrates that ionizing radiation leads to an increased expression in IL-6 and intercellular adhesion molecule 1 (ICAM1) mRNA protein level.

FIG. 16 demonstrates a putative neuroprotective mechanism through anti-inflammation by illustrating activation of nAchR-α7 to abolish radiation-induced upregulation of IL-6 and ICAM1.

FIG. 17 demonstrates a putative neuroprotective mechanism through anti-inflammation by illustrating the effects of preincubation with an α7 antagonist.

FIG. 18 depicts a putative neuroprotective mechanism through anti-inflammation.

FIG. 19 demonstrates a putative neuroprotective mechanism through anti-inflammation by illustrating activation of nAChR-α7 to modulate radiation induced inflammation responses.

FIG. 20 demonstrates a putative neuroprotective mechanism through anti-inflammation by illustrating activation of nAChR-α7 to restore radiation-induced levels of mitochondrial proteins.

FIG. 21 demonstrates improved cognition as illustrated through activation of nAChR-α7.

FIG. 22 depicts the modulation of a radiation-induced inflammatory response.

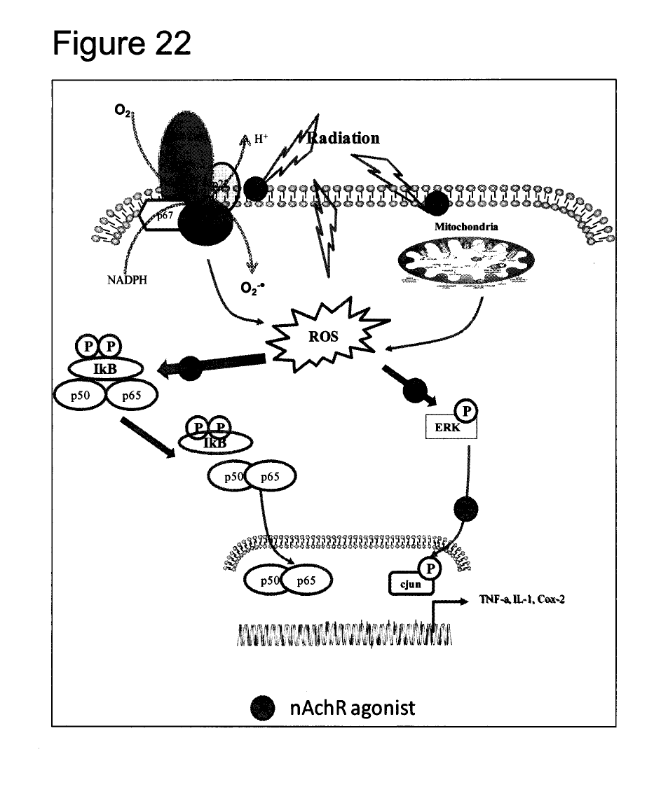

### DETAILED DESCRIPTION OF THE PREFERRED EMBODIMENTS

The following definitions are meant to refine but not limit, the terms defined. If a particular term used herein is not specifically defined, such term should not be considered indefinite. Rather, terms are used within their accepted meanings.

As used throughout this specification, the preferred number of atoms, such as carbon atoms, will be represented by, for example, the phrase “Cₓ—C_{y} alkyl,” which refers to an alkyl group, as herein defined, containing the specified number of carbon atoms. Similar terminology will apply for other preferred terms and ranges as well. One embodiment of the present invention includes so-called ‘lower’ alkyl chains of one to eight, preferably one to six carbon atoms. Thus, for example, C₁-C₆ alkyl represents a lower alkyl chain as hereinabove described. As used herein the term “alkyl” refers to a straight or branched chain hydrocarbon having one to eight carbon atoms, preferably one to six carbon atoms, which may be optionally substituted as herein further described, with multiple degrees of substitution being allowed. Examples of “alkyl” as used herein include, but are not limited to, methyl, ethyl, propyl, isopropyl, isobutyl, n-butyl, tert-butyl, isopentyl, and n-pentyl.

As used herein the term “alkenyl” refers to a straight or branched chain aliphatic hydrocarbon having two to twelve carbon atoms, preferably two to eight carbon atoms, and containing one or more carbon-to-carbon double bonds, which may be optionally substituted as herein further described, with multiple degrees of substitution being allowed. Examples of “alkenyl” as used herein include, but are not limited to, vinyl, and allyl.

As used herein the term “alkynyl” refers to a straight or branched chain aliphatic hydrocarbon having two to twelve carbon atoms, preferably two to eight carbon atoms, and containing one or more carbon-to-carbon triple bonds, which may be optionally substituted as herein further described, with multiple degrees of substitution being allowed. An example of “alkynyl” as used herein includes, but is not limited to, ethynyl.

As used herein, the term “cycloalkyl” refers to a fully saturated optionally substituted three- to twelve-membered, preferably three- to eight-membered, monocyclic, bicyclic, or bridged hydrocarbon ring, with multiple degrees of substitution being allowed. Exemplary “cycloalkyl” groups as used herein include, but are not limited to, cyclopropyl, cyclobutyl, cyclopentyl, cyclohexyl, and cycloheptyl.

Similarly, as used herein, the terms “cycloalkenyl” and “cycloalkynyl” refer to optionally substituted, partially saturated but non-aromatic, three-to-twelve membered, preferably either five- to eight-membered or seven- to ten-membered, monocyclic, bicyclic, or bridged hydrocarbon rings, with one or more degrees of unsaturation, and with multiple degrees of substitution being allowed.

As used herein, the term “heterocycle” or “heterocyclyl” refers to an optionally substituted mono- or polycyclic ring system, optionally containing one or more degrees of unsaturation and also containing one or more heteroatoms, which may be optionally substituted as herein further described, with multiple degrees of substitution being allowed. Exemplary heteroatoms include nitrogen, oxygen, or sulfur atoms, including N-oxides, sulfur oxides, and dioxides. Preferably, the ring is three to twelve-membered, preferably three- to eight-membered and is either fully saturated or has one or more degrees of unsaturation. Such rings may be optionally fused to one or more of another heterocyclic ring(s) or cycloalkyl ring(s). Examples of “heterocyclic” groups as used herein include, but are not limited to, tetrahydrofuran, pyran, 1,4-dioxane, 1,3-dioxane, piperidine, pyrrolidine, morpholine, tetrahydrothiopyran, and tetrahydrothiophene.

As used herein, the term “aryl” refers to a univalent benzene ring or fused benzene ring system, which may be optionally substituted as herein further described, with multiple degrees of substitution being allowed. Examples of “aryl” groups as used include, but are not limited to, phenyl, 2-naphthyl, 1-naphthyl, anthracene, and phenanthrene. Preferable aryl rings have five- to ten-members.

As used herein, a fused benzene ring system encompassed within the term “aryl” includes fused polycyclic hydrocarbons, namely where a cyclic hydrocarbon with less than maximum number of noncumulative double bonds, for example where a saturated hydrocarbon ring (cycloalkyl, such as a cyclopentyl ring) is fused with an aromatic ring (aryl, such as a benzene ring) to form, for example, groups such as indanyl and acenaphthalenyl, and also includes such groups as, for non-limiting examples, dihydronaphthalene and hexahydrocyclopenta-cyclooctene.

As used herein, the term “aralkyl” refers to an “aryl” group as herein defined attached through an alkylene linker.

As used herein, the term “heteroaryl” refers to a monocyclic five to seven membered aromatic ring, or to a fused bicyclic aromatic ring system comprising two of such aromatic rings, which may be optionally substituted as herein further described, with multiple degrees of substitution being allowed. Preferably, such rings contain five- to ten-members. These heteroaryl rings contain one or more nitrogen, sulfur, and oxygen atoms, where N-oxides, sulfur oxides, and dioxides are permissible heteroatom substitutions. Examples of “heteroaryl” groups as used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzoxazole, benzothiophene, indole, indazole, benzimidazole, imidazopyridine, pyrazolopyridine, and pyrazolopyrimidine.

As used herein, the term “heteroaralkyl” refers to an “heteroaryl” group as herein defined attached through an alkylene linker.

As used herein the term “halogen” refers to fluorine, chlorine, bromine, or iodine.

As used herein the term “haloalkyl” refers to an alkyl group, as defined herein, that is substituted with at least one halogen. Examples of branched or straight chained “haloalkyl” groups as used herein include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups such as —CF₃.

As used herein the term “alkoxy” refers to a group —OR^{a}, where R^{a} is alkyl as defined above.

As used herein the term “nitro” refers to a group —NO₂.

As used herein the term “cyano” refers to a group —CN.

As used herein the term “azido” refers to a group —N₃.

As used herein “amino” refers to a group —NR^{a}R^{b}, where each of R^{a} and R^{b} individually is hydrogen, alkyl, alkenyl, alkynyl, cycloalkyl, aryl, heterocylcyl, or heteroaryl. As used herein, when either R^{a} or R^{b} is other than hydrogen, such a group may be referred to as a “substituted amino” or, for example if R^{a} is H and R^{b} is alkyl, as an “alkylamino.”

As used herein, the term “hydroxyl” refers to a group —OH.

The physiologic effects of alpha7 nAChR agonists include neurogenesis and protection against neuro-inflammation and subsequent damage. Thus, one aspect of the present invention is selective alpha7 nAChR agonist compounds for treating or preventing disorders and conditions for which recruitment of neurogenesis is potentially therapeutic. The physiologic effects of alpha4beta2 nAChR agonists include neuroprotection. Thus another aspect of the present invention is a combination of an alpha4beta2 agonist and an alpha7 agonist, or a single agonist with dual alpha7/alpha4beta2 pharmacology for use in prevention or treatment of conditions such as “chemobrain,” chemotherapy-induced cognitive deficits, radiation-induced cognitive deficits, ischemic events, autoimmune CNS disorders, and a variety of other neurodegenerative disorders, especially those that involve neuro-inflammation. Moreover, a combination therapy of an alpha4beta2 antagonist, for correction of hypercholinergic tone, and an alpha7 agonist (for neurogenesis) would be expected to address both the symptoms and the underlying cause of major depressive disorder and brain reward disorder indications. Thus another aspect of the present invention is a combination of an alpha4beta2 antagonist and an alpha7 agonist, or a “dual” compound of similar pharmacology for treatment of major depressive disorder, addictions, dysregulated food intake, and bipolar disorder. Yet another aspect of the invention is the use of alpha7 antagonists in adjunct therapy (with radiation) for treatment of GBM. Yet another aspect of the invention is the use of both an alpha7 agonist (to protect healthy tissue from damage) and an alpha7 antagonist (to enhance the effectiveness of the radiation) in one of various combinations, representing various options for site and timing of administration.

As used herein, the terms “prevention” or “prophylaxis” include any degree of reducing the progression of or delaying the onset of a disease, disorder, or condition. The term includes providing protective effects against a particular disease, disorder, or condition as well as amelioration of the recurrence of the disease, disorder, or condition.

Compound A is (5-methyl-N-[(2S,3R)-2-(pyridin-3-ylmethyl)-1-azabicyclo[2.2.2]oct-3-yl]thiophene-2-carboxamide) or a pharmaceutically acceptable salt thereof, illustrated below.

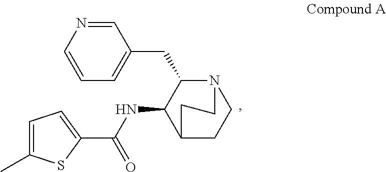

or a pharmaceutically acceptable salt thereof.
   As will be appreciated, different naming conventions provide alternative names. Thus, Compound A may also be referred to as (2S,3R)-N-(2-((3-pyridinyl)methyl)-1-azabicyclo[2.2.2]octan-3-yl)-5-methylthiophene-2-carboxamide. Such naming conventions should not impact the clarity of the present invention.

### Compounds

Compounds useful according to the present invention are alpha7 NNR selective ligands, as exemplified by Compound A herein.

Compound A is a member of a genus of compounds described in U.S. Pat. No. 6,953,855 (incorporated herein by reference in its entirety). U.S. Pat. No. 6,953,855 includes compounds represented by Formula 1.

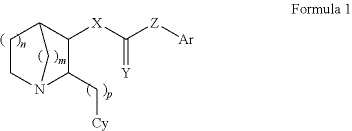

In Formula I, m and n individually can have a value of 1 or 2, and p can have a value of 1, 2, 3 or 4. In the Formula, X is either oxygen or nitrogen (i.e., NR′), Y is either oxygen or sulfur, and Z is either nitrogen (i.e., NR′), a covalent bond, or a linker species, A. A is selected from the group —CR′R″—, —CR′R″—CR′R″—, —CR′═CR′—, and —C₂—, wherein R′ and R″ are as hereinafter defined. When Z is a covalent bond or A, X must be nitrogen. Ar is an aryl group, either carbocyclic or heterocyclic, either monocyclic or fused polycyclic, unsubstituted or substituted; and Cy is a 5- or 6-membered heteroaromatic ring, unsubstituted or substituted. Thus, the invention includes compounds in which Ar is linked to the azabicycle by a carbonyl group-containing functionality, such as an amide, carbamate, urea, thioamide, thiocarbamate, or thiourea functionality. In addition, in the case of the amide and thioamide functionalities, Ar may be bonded directly to the carbonyl (or thiocarbonyl) group or may be linked to the carbonyl (or thiocarbonyl) group through linker A. Furthermore, the invention includes compounds that contain a 1-azabicycle, containing either a 5-, 6-, or 7-membered ring, and having a total of 7, 8 or 9 ring atoms (e.g., 1-azabicyclo[2.2.1]heptane, 1-azabicyclo[3.2.1]octane, 1-azabicyclo[2.2.2]octane, and 1-azabicyclo[3.2.2]nonane).

In one embodiment, the value of p is 1, Cy is 3-pyridinyl or 5-pyrimidinyl, X and Y are oxygen, and Z is nitrogen. In another embodiment, the value of p is 1, Cy is 3-pyridinyl or 5-pyrimidinyl, X and Z are nitrogen, and Y is oxygen. In a third embodiment, the value of p is 1, Cy is 3-pyridinyl or 5-pyrimidinyl, X is nitrogen, Y is oxygen, and Z is a covalent bond (between the carbonyl and Ar). In a fourth embodiment, the value of p is 1, Cy is 3-pyridinyl or 5-pyrimidinyl, X is nitrogen, Y is oxygen, Z is A (a linker species between the carbonyl and Ar).

The compounds of Formula 1 have one or more asymmetric carbons and can therefore exist in the form of racemic mixtures, enantiomers, and diastereomers. Both relative and absolute stereochemistry at asymmetric carbons are variable (e.g., cis or trans, R or S). In addition, some of the compounds exist as E and Z isomers about a carbon-carbon double bond. All these individual isomeric compounds and their mixtures are also intended to be within the scope of Formula 1.

As used in Formula 1, Ar (“aryl”) includes both carbocyclic and heterocyclic aromatic rings, both monocyclic and fused polycyclic, where the aromatic rings can be 5- or 6-membered rings. Representative monocyclic aryl groups include, but are not limited to, phenyl, furanyl, pyrrolyl, thienyl, pyridinyl, pyrimidinyl, oxazolyl, isoxazolyl, pyrazolyl, imidazolyl, thiazolyl, isothiazolyl, and the like. Fused polycyclic aryl groups are those aromatic groups that include a 5- or 6-membered aromatic or heteroaromatic ring as one or more rings in a fused ring system. Representative fused polycyclic aryl groups include naphthalene, anthracene, indolizine, indole, isoindole, benzofuran, benzothiophene, indazole, benzimidazole, benzthiazole, purine, quinoline, isoquinoline, cinnoline, phthalazine, quinazoline, quinoxaline, 1,8-naphthyridine, pteridine, carbazole, acridine, phenazine, phenothiazine, phenoxazine, and azulene.

As used in Formula 1, “Cy” groups are 5- and 6-membered ring heteroaromatic groups. Representative Cy groups include pyridinyl, pyrimidinyl, furanyl, pyrrolyl, thienyl, oxazolyl, isoxazolyl, pyrazolyl, imidazolyl, thiazolyl, isothiazolyl, and the like, where pyridinyl is preferred.

Individually, Ar and Cy can be unsubstituted or can be substituted with 1, 2, or 3 substituents, such as alkyl, alkenyl, heterocyclyl, cycloalkyl, aryl, substituted aryl, arylalkyl, substituted arylalkyl, halo (e.g., F, Cl, Br, or I), —OR′, —NR′R″, —CF₃, —CN, —NO₂, —C₂R′, —SR′, —N₃, —C(═O)NR′R″, —NR′C(═O)R″, —C(═O)R′, —C(═O)OR′, —OC(═O)R′, —O(CR′R″)ᵣC(═O)R′, —O(CR′R″)ᵣNR″C(═O)R′, —O(CR′R″)ᵣNR″SO₂R′, —OC(═O)NR′R″, —NRC(═O)OR″, —SO₂R′, —SO₂NR′R″, and —NR′SO₂R″, where R′ and R″ are individually hydrogen, C₁-C₈ alkyl (e.g., straight chain or branched alkyl, preferably C₁-C₈, such as methyl, ethyl, or isopropyl), cycloalkyl (e.g., C₃₋₅ cyclic alkyl), heterocyclyl, aryl, or arylalkyl (such as benzyl), and r is an integer from 1 to 6. R′ and R″ can also combine to form a cyclic functionality.

Compounds of Formula 1 form acid addition salts which are useful according to the present invention. Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate. The salts may be in some cases hydrates or ethanol solvates.

Representative compounds of Formula 1 include:
2-((3-pyridinyl)methyl)-1-azabicyclo[2.2.2]oct-3-yl N-phenylcarbamate,
2-((3-pyridinyl)methyl)-1-azabicyclo[2.2.2]oct-3-yl N-(4-fluorophenyl)carbamate,
2-((3-pyridinyl)methyl)-1-azabicyclo[2.2.2]oct-3-yl N-(4-chlorophenyl)carbamate,
2-((3-pyridinyl)methyl)-1-azabicyclo[2.2.2]oct-3-yl N-(4-bromophenyl)carbamate,
2-((3-pyridinyl)methyl)-1-azabicyclo[2.2.2]oct-3-yl N-(3-fluorophenyl)carbamate,
2-((3-pyridinyl)methyl)-1-azabicyclo[2.2.2]oct-3-yl N-(3-chlorophenyl)carbamate,
2-((3-pyridinyl)methyl)-1-azabicyclo[2.2.2]oct-3-yl N-(3-bromophenyl)carbamate,
2-((3-pyridinyl)methyl)-1-azabicyclo[2.2.2]oct-3-yl N-(2-fluorophenyl)carbamate,
2-((3-pyridinyl)methyl)-1-azabicyclo[2.2.2]oct-3-yl N-(2-chlorophenyl)carbamate,
2-((3-pyridinyl)methyl)-1-azabicyclo[2.2.2]oct-3-yl N-(2-bromophenyl)carbamate,
2-((3-pyridinyl)methyl)-1-azabicyclo[2.2.2]oct-3-yl N-(3,4-dichlorophenyl)carbamate,
2-((3-pyridinyl)methyl)-1-azabicyclo[2.2.2]oct-3-yl N-(2-methylphenyl)carbamate,
2-((3-pyridinyl)methyl)-1-azabicyclo[2.2.2]oct-3-yl N-(2-biphenyl)carbamate,
2-((3-pyridinyl)methyl)-1-azabicyclo[2.2.2]oct-3-yl N-(3-methylphenyl)carbamate,
2-((3-pyridinyl)methyl)-1-azabicyclo[2.2.2]oct-3-yl N-(3-biphenyl)carbamate,
2-((3-pyridinyl)methyl)-1-azabicyclo[2.2.2]oct-3-yl N-(4-methylphenyl)carbamate,
2-((3-pyridinyl)methyl)-1-azabicyclo[2.2.2]oct-3-yl N-(4-biphenyl)carbamate,
2-((3-pyridinyl)methyl)-1-azabicyclo[2.2.2]oct-3-yl N-(2-cyanophenyl)carbamate,
2-((3-pyridinyl)methyl)-1-azabicyclo[2.2.2]oct-3-yl N-(3-cyanophenyl)carbamate,
2-((3-pyridinyl)methyl)-1-azabicyclo[2.2.2]oct-3-yl N-(4-cyanophenyl)carbamate,
2-((3-pyridinyl)methyl)-1-azabicyclo[2.2.2]oct-3-yl N-(3-trifluoromethylphenyl)carbamate,
2-((3-pyridinyl)methyl)-1-azabicyclo[2.2.2]oct-3-yl N-(4-dimethylaminophenyl)carbamate,
2-((3-pyridinyl)methyl)-1-azabicyclo[2.2.2]oct-3-yl N-(2-methoxyphenyl)carbamate,
2-((3-pyridinyl)methyl)-1-azabicyclo[2.2.2]oct-3-yl N-(2-phenoxyphenyl)carbamate,
2-((3-pyridinyl)methyl)-1-azabicyclo[2.2.2]oct-3-yl N-(2-methylthiophenyl)carbamate,
2-((3-pyridinyl)methyl)-1-azabicyclo[2.2.2]oct-3-yl N-(2-phenylthiophenyl)carbamate,
2-((3-pyridinyl)methyl)-1-azabicyclo[2.2.2]oct-3-yl N-(3-methoxyphenyl)carbamate,
2-((3-pyridinyl)methyl)-1-azabicyclo[2.2.2]oct-3-yl N-(3-phenoxyphenyl)carbamate,
2-((3-pyridinyl)methyl)-1-azabicyclo[2.2.2]oct-3-yl N-(3-methylthiophenyl)carbamate,
2-((3-pyridinyl)methyl)-1-azabicyclo[2.2.2]oct-3-yl N-(3-phenylthiophenyl)carbamate,
2-((3-pyridinyl)methyl)-1-azabicyclo[2.2.2]oct-3-yl N-(4-methoxyphenyl)carbamate,
2-((3-pyridinyl)methyl)-1-azabicyclo[2.2.2]oct-3-yl N-(4-phenoxyphenyl)carbamate,
2-((3-pyridinyl)methyl)-1-azabicyclo[2.2.2]oct-3-yl N-(4-methylthiophenyl)carbamate,
2-((3-pyridinyl)methyl)-1-azabicyclo[2.2.2]oct-3-yl N-(4-phenylthiophenyl)carbamate,
2-((3-pyridinyl)methyl)-1-azabicyclo[2.2.2]oct-3-yl N-(2,4-dimethoxyphenyl)carbamate,
2-((3-pyridinyl)methyl)-1-azabicyclo[2.2.2]oct-3-yl N-(2-thienyl)carbamate,
2-((3-pyridinyl)methyl)-1-azabicyclo[2.2.2]oct-3-yl N-(3-thienyl)carbamate,
2-((3-pyridinyl)methyl)-1-azabicyclo[2.2.2]oct-3-yl N-(3-benzothienyl)carbamate,
2-((3-pyridinyl)methyl)-1-azabicyclo[2.2.2]oct-3-yl N-(1-naphthyl)carbamate, and
2-((3-pyridinyl)methyl)-1-azabicyclo[2.2.2]oct-3-yl N-(2-naphthyl)carbamate,
   or a pharmaceutically acceptable salt thereof.

Other compounds representative of Formula 1 include:
N-phenyl-N′-(2-((3-pyridinyl)methyl)-1-azabicyclo[2.2.2]oct-3-yl)urea,
N-(4-fluorophenyl)-N′-(2-((3-pyridinyl)methyl)-1-azabicyclo[2.2.2]oct-3-yl)urea,
N-(4-chlorophenyl)-N′-(2-((3-pyridinyl)methyl)-1-azabicyclo[2.2.2]oct-3-yl)urea,
N-(4-bromophenyl)-N′-(2-((3-pyridinyl)methyl)-1-azabicyclo[2.2.2]oct-3-yl)urea,
N-(3-fluorophenyl)-N′-(2-((3-pyridinyl)methyl)-1-azabicyclo[2.2.2]oct-3-yl)urea,
N-(3-chlorophenyl)-N′-(2-((3-pyridinyl)methyl)-1-azabicyclo[2.2.2]oct-3-yl)urea,
N-(3-bromophenyl)-N′-(2-((3-pyridinyl)methyl)-1-azabicyclo[2.2.2]oct-3-yl)urea,
N-(2-fluorophenyl)-N′-(2-((3-pyridinyl)methyl)-1-azabicyclo[2.2.2]oct-3-yl)urea,
N-(2-chlorophenyl)-N′-(2-((3-pyridinyl)methyl)-1-azabicyclo[2.2.2]oct-3-yl)urea,
N-(2-bromophenyl)-N′-(2-((3-pyridinyl)methyl)-1-azabicyclo[2.2.2]oct-3-yl)urea,
N-(3,4-dichlorophenyl)-N′-(2-((3-pyridinyl)methyl)-1-azabicyclo[2.2.2]oct-3-yl)urea,
N-(2-methylphenyl)-N′-(2-((3-pyridinyl)methyl)-1-azabicyclo[2.2.2]oct-3-yl)urea,
N-(2-biphenyl)-N′-(2-((3-pyridinyl)methyl)-1-azabicyclo[2.2.2]oct-3-yl)urea,
N-(3-methylphenyl)-N′-(2-((3-pyridinyl)methyl)-1-azabicyclo[2.2.2]oct-3-yl)urea,
N-(3-biphenyl)-N′-(2-((3-pyridinyl)methyl)-1-azabicyclo[2.2.2]oct-3-yl)urea,
N-(4-methylphenyl)-N′-(2-((3-pyridinyl)methyl)-1-azabicyclo[2.2.2]oct-3-yl)urea,
N-(4-biphenyl)-N′-(2-((3-pyridinyl)methyl)-1-azabicyclo[2.2.2]oct-3-yl)urea,
N-(2-cyanophenyl)-N′-(2-((3-pyridinyl)methyl)-1-azabicyclo[2.2.2]oct-3-yl)urea,
N-(3-cyanophenyl)-N′-(2-((3-pyridinyl)methyl)-1-azabicyclo[2.2.2]oct-3-yl)urea,
N-(4-cyanophenyl)-N′-(2-((3-pyridinyl)methyl)-1-azabicyclo[2.2.2]oct-3-yl)urea,
N-(3-trifluoromethylphenyl)-N′-(2-((3-pyridinyl)methyl)-1-azabicyclo[2.2.2]oct-3-yl)urea,
N-(4-dimethylaminophenyl)-N′-(2-((3-pyridinyl)methyl)-1-azabicyclo[2.2.2]oct-3-yl)urea,
N-(2-methoxyphenyl)-N′-(2-((3-pyridinyl)methyl)-1-azabicyclo[2.2.2]oct-3-yl)urea,
N-(2-phenoxyphenyl)-N′-(2-((3-pyridinyl)methyl)-1-azabicyclo[2.2.2]oct-3-yl)urea,
N-(2-methylthiophenyl)-N′-(2-((3-pyridinyl)methyl)-1-azabicyclo[2.2.2]oct-3-yl)urea,
N-(2-phenylthiophenyl)-N′-(2-((3-pyridinyl)methyl)-1-azabicyclo[2.2.2]oct-3-yl)urea,
N-(3-methoxyphenyl)-N′-(2-((3-pyridinyl)methyl)-1-azabicyclo[2.2.2]oct-3-yl)urea,
N-(3-phenoxyphenyl)-N′-(2-((3-pyridinyl)methyl)-1-azabicyclo[2.2.2]oct-3-yl)urea,
N-(3-methylthiophenyl)-N′-(2-((3-pyridinyl)methyl)-1-azabicyclo[2.2.2]oct-3-yl)urea,
N-(3-phenylthiophenyl)-N′-(2-((3-pyridinyl)methyl)-1-azabicyclo[2.2.2]oct-3-yl)urea,
N-(4-methoxyphenyl)-N′-(2-((3-pyridinyl)methyl)-1-azabicyclo[2.2.2]oct-3-yl)urea,
N-(4-phenoxyphenyl)-N′-(2-((3-pyridinyl)methyl)-1-azabicyclo[2.2.2]oct-3-yl)urea,
N-(4-methylthiophenyl)-N′-(2-((3-pyridinyl)methyl)-1-azabicyclo[2.2.2]oct-3-yl)urea,
N-(4-phenylthiophenyl)-N′-(2-((3-pyridinyl)methyl)-1-azabicyclo[2.2.2]oct-3-yl)urea,
N-(2,4-dimethoxyphenyl)-N′-(2-((3-pyridinyl)methyl)-1-azabicyclo[2.2.2]oct-3-yl)urea,
N-(2-thienyl)-N′-(2-((3-pyridinyl)methyl)-1-azabicyclo[2.2.2]oct-3-yl)urea,
N-(3-thienyl)-N′-(2-((3-pyridinyl)methyl)-1-azabicyclo[2.2.2]oct-3-yl)urea,
N-(3-benzothienyl)-N′-(2-((3-pyridinyl)methyl)-1-azabicyclo[2.2.2]oct-3-yl)urea,
N-(1-naphthyl)-N′-(2-((3-pyridinyl)methyl)-1-azabicyclo[2.2.2]oct-3-yl)urea, and
N-(2-naphthyl)-N′-(2-((3-pyridinyl)methyl)-1-azabicyclo[2.2.2]oct-3-yl)urea,
   or a pharmaceutically acceptable salt thereof.

Other compounds representative of Formula 1 include:
N-(2-((3-pyridinyl)methyl)-1-azabicyclo[2.2.2]oct-3-yl)benzamide,
N-(2-((3-pyridinyl)methyl)-1-azabicyclo[2.2.2]oct-3-yl)-2-fluorobenzamide,
N-(2-((3-pyridinyl)methyl)-1-azabicyclo[2.2.2]oct-3-yl)-3-fluorobenzamide,
N-(2-((3-pyridinyl)methyl)-1-azabicyclo[2.2.2]oct-3-yl)-4-fluorobenzamide,
N-(2-((3-pyridinyl)methyl)-1-azabicyclo[2.2.2]oct-3-yl)-2-chlorobenzamide,
N-(2-((3-pyridinyl)methyl)-1-azabicyclo[2.2.2]oct-3-yl)-3-chlorobenzamide,
N-(2-((3-pyridinyl)methyl)-1-azabicyclo[2.2.2]oct-3-yl)-4-chlorobenzamide,
N-(2-((3-pyridinyl)methyl)-1-azabicyclo[2.2.2]oct-3-yl)-2-bromobenzamide,
N-(2-((3-pyridinyl)methyl)-1-azabicyclo[2.2.2]oct-3-yl)-3-bromobenzamide,
N-(2-((3-pyridinyl)methyl)-1-azabicyclo[2.2.2]oct-3-yl)-4-bromobenzamide,
N-(2-((3-pyridinyl)methyl)-1-azabicyclo[2.2.2]oct-3-yl)-3,4-dichlorobenzamide,
N-(2-((3-pyridinyl)methyl)-1-azabicyclo[2.2.2]oct-3-yl)-2-methylbenzamide,
N-(2-((3-pyridinyl)methyl)-1-azabicyclo[2.2.2]oct-3-yl)-3-methylbenzamide,
N-(2-((3-pyridinyl)methyl)-1-azabicyclo[2.2.2]oct-3-yl)-4-methylbenzamide,
N-(2-((3-pyridinyl)methyl)-1-azabicyclo[2.2.2]oct-3-yl)-2-phenylbenzamide,
N-(2-((3-pyridinyl)methyl)-1-azabicyclo[2.2.2]oct-3-yl)-3-phenylbenzamide,
N-(2-((3-pyridinyl)methyl)-1-azabicyclo[2.2.2]oct-3-yl)-4-phenylbenzamide,
N-(2-((3-pyridinyl)methyl)-1-azabicyclo[2.2.2]oct-3-yl)-2-cyanobenzamide,
N-(2-((3-pyridinyl)methyl)-1-azabicyclo[2.2.2]oct-3-yl)-3-cyanobenzamide,
N-(2-((3-pyridinyl)methyl)-1-azabicyclo[2.2.2]oct-3-yl)-4-cyanobenzamide,
N-(2-((3-pyridinyl)methyl)-1-azabicyclo[2.2.2]oct-3-yl)-3-trifluoromethylbenzamide,
N-(2-((3-pyridinyl)methyl)-1-azabicyclo[2.2.2]oct-3-yl)-4-dimethylaminobenzamide,
N-(2-((3-pyridinyl)methyl)-1-azabicyclo[2.2.2]oct-3-yl)-2-methoxybenzamide,
N-(2-((3-pyridinyl)methyl)-1-azabicyclo[2.2.2]oct-3-yl)-3-methoxybenzamide,
N-(2-((3-pyridinyl)methyl)-1-azabicyclo[2.2.2]oct-3-yl)-4-methoxybenzamide,
N-(2-((3-pyridinyl)methyl)-1-azabicyclo[2.2.2]oct-3-yl)-2-phenoxybenzamide,
N-(2-((3-pyridinyl)methyl)-1-azabicyclo[2.2.2]oct-3-yl)-3-phenoxybenzamide,
N-(2-((3-pyridinyl)methyl)-1-azabicyclo[2.2.2]oct-3-yl)-4-phenoxybenzamide,
N-(2-((3-pyridinyl)methyl)-1-azabicyclo[2.2.2]oct-3-yl)-2-methylthiobenzamide,
N-(2-((3-pyridinyl)methyl)-1-azabicyclo[2.2.2]oct-3-yl)-3-methylthiobenzamide,
N-(2-((3-pyridinyl)methyl)-1-azabicyclo[2.2.2]oct-3-yl)-4-methylthiobenzamide,
N-(2-((3-pyridinyl)methyl)-1-azabicyclo[2.2.2]oct-3-yl)-2-phenylthiobenzamide,
N-(2-((3-pyridinyl)methyl)-1-azabicyclo[2.2.2]oct-3-yl)-3-phenylthiobenzamide,
N-(2-((3-pyridinyl)methyl)-1-azabicyclo[2.2.2]oct-3-yl)-4-phenylthiobenzamide,
N-(2-((3-pyridinyl)methyl)-1-azabicyclo[2.2.2]oct-3-yl)-2,4-dimethoxybenzamide,
N-(2-((3-pyridinyl)methyl)-1-azabicyclo[2.2.2]oct-3-yl)-5-bromonicotinamide,
N-(2-((3-pyridinyl)methyl)-1-azabicyclo[2.2.2]oct-3-yl)-6-chloronicotinamide,
N-(2-((3-pyridinyl)methyl)-1-azabicyclo[2.2.2]oct-3-yl)-5-phenylnicotinamide,
N-(2-((3-pyridinyl)methyl)-1-azabicyclo[2.2.2]oct-3-yl)furan-2-carboxamide,
N-(2-((3-pyridinyl)methyl)-1-azabicyclo[2.2.2]oct-3-yl)furan-3-carboxamide,
N-(2-((3-pyridinyl)methyl)-1-azabicyclo[2.2.2]oct-3-yl)thiophene-2-carboxamide,
N-(2-((3-pyridinyl)methyl)-1-azabicyclo[2.2.2]oct-3-yl)-5-bromothiophene-2-carboxamide,
N-(2-((3-pyridinyl)methyl)-1-azabicyclo[2.2.2]oct-3-yl)-5-methylthiothiophene-2-carboxamide,
N-(2-((3-pyridinyl)methyl)-1-azabicyclo[2.2.2]oct-3-yl)-5-phenylthiothiophene-2-carboxamide,
N-(2-((3-pyridinyl)methyl)-1-azabicyclo[2.2.2]oct-3-yl)-5-methylthiophene-2-carboxamide,
N-(2-((3-pyridinyl)methyl)-1-azabicyclo[2.2.2]oct-3-yl)-3-methylthiophene-2-carboxamide,
N-(2-((3-pyridinyl)methyl)-1-azabicyclo[2.2.2]oct-3-yl)-3-bromothiophene-2-carboxamide,
N-(2-((3-pyridinyl)methyl)-1-azabicyclo[2.2.2]oct-3-yl)-3-chlorothiophene-2-carboxamide,
N-(2-((3-pyridinyl)methyl)-1-azabicyclo[2.2.2]oct-3-yl)-5-(2-pyridinyl)thiophene-2-carboxamide,
N-(2-((3-pyridinyl)methyl)-1-azabicyclo[2.2.2]oct-3-yl)-5-acetylthiophene-2-carboxamide,
N-(2-((3-pyridinyl)methyl)-1-azabicyclo[2.2.2]oct-3-yl)-3-ethoxythiophene-2-carboxamide,
N-(2-((3-pyridinyl)methyl)-1-azabicyclo[2.2.2]oct-3-yl)-3-methoxythiophene-2-carboxamide,
N-(2-((3-pyridinyl)methyl)-1-azabicyclo[2.2.2]oct-3-yl)-4-acetyl-3-methyl-5-methylthiothiophene-2-carboxamide,
N-(2-((3-pyridinyl)methyl)-1-azabicyclo[2.2.2]oct-3-yl)thiophene-3-carboxamide,
N-(2-((3-pyridinyl)methyl)-1-azabicyclo[2.2.2]oct-3-yl)-1-methylpyrrole-2-carboxamide,
N-(2-((3-pyridinyl)methyl)-1-azabicyclo[2.2.2]oct-3-yl)pyrrole-3-carboxamide,
N-(2-((3-pyridinyl)methyl)-1-azabicyclo[2.2.2]oct-3-yl)indole-2-carboxamide,
N-(2-((3-pyridinyl)methyl)-1-azabicyclo[2.2.2]oct-3-yl)indole-3-carboxamide,
N-(2-((3-pyridinyl)methyl)-1-azabicyclo[2.2.2]oct-3-yl)-1-methylindole-3-carboxamide,
N-(2-((3-pyridinyl)methyl)-1-azabicyclo[2.2.2]oct-3-yl)-1-benzylindole-3-carboxamide,
N-(2-((3-pyridinyl)methyl)-1-azabicyclo[2.2.2]oct-3-yl)-1H-benzimidazole-2-carboxamide,
N-(2-((3-pyridinyl)methyl)-1-azabicyclo[2.2.2]oct-3-yl)-1-isopropyl-2-trifluoromethyl-1H-benzimidazole-5-carboxamide,
N-(2-((3-pyridinyl)methyl)-1-azabicyclo[2.2.2]oct-3-yl)-1-isopropyl-1H-benzotriazole-5-carboxamide,
N-(2-((3-pyridinyl)methyl)-1-azabicyclo[2.2.2]oct-3-yl)benzo[b]thiophene-2-carboxamide,
N-(2-((3-pyridinyl)methyl)-1-azabicyclo[2.2.2]oct-3-yl)benzo[b]thiophene-3-carboxamide,
N-(2-((3-pyridinyl)methyl)-1-azabicyclo[2.2.2]oct-3-yl)benzofuran-2-carboxamide,
N-(2-((3-pyridinyl)methyl)-1-azabicyclo[2.2.2]oct-3-yl)benzofuran-3-carboxamide,
N-(2-((3-pyridinyl)methyl-1-azabicyclo[2.2.2]oct-3-yl)-3-methylbenzofuran-2-carboxamide,
N-(2-((3-pyridinyl)methyl-1-azabicyclo[2.2.2]oct-3-yl)-5-nitrobenzofuran-2-carboxamide,
N-(2-((3-pyridinyl)methyl-1-azabicyclo[2.2.2]oct-3-yl)-5-methoxybenzofuran-2-carboxamide,
N-(2-((3-pyridinyl)methyl-1-azabicyclo[2.2.2]oct-3-yl)-7-methoxybenzofuran-2-carboxamide,
N-(2-((3-pyridinyl)methyl-1-azabicyclo[2.2.2]oct-3-yl)-7-ethoxybenzofuran-2-carboxamide,
N-(2-((3-pyridinyl)methyl-1-azabicyclo[2.2.2]oct-3-yl)-3-methyl-5-chlorobenzofuran-2-carboxamide,
N-(2-((3-pyridinyl)methyl-1-azabicyclo[2.2.2]oct-3-yl)-6-bromobenzofuran-2-carboxamide,
N-(2-((3-pyridinyl)methyl-1-azabicyclo[2.2.2]oct-3-yl)-4-acetyl-7-methoxybenzofuran-2-carboxamide,
N-(2-((3-pyridinyl)methyl-1-azabicyclo[2.2.2]oct-3-yl)-2-methylbenzofuran-4-carboxamide,
N-(2-((3-pyridinyl)methyl-1-azabicyclo[2.2.2]oct-3-yl)naphtho[2,1-b]furan-2-carboxamide,
N-(2-((3-pyridinyl)methyl)-1-azabicyclo[2.2.2]oct-3-yl)naphthalene-1-carboxamide,
N-(2-((3-pyridinyl)methyl)-1-azabicyclo[2.2.2]oct-3-yl)naphthalene-2-carboxamide,
N-(2-((3-pyridinyl)methyl)-1-azabicyclo[2.2.2]oct-3-yl)-6-aminonaphthalene-2-carboxamide,
N-(2-((3-pyridinyl)methyl-1-azabicyclo[2.2.2]oct-3-yl)-3-methoxynaphthalene-2-carboxamide,
N-(2-((3-pyridinyl)methyl-1-azabicyclo[2.2.2]oct-3-yl)-6-methoxynaphthalene-2-carboxamide,
N-(2-((3-pyridinyl)methyl-1-azabicyclo[2.2.2]oct-3-yl)-1-hydroxynaphthalene-2-carboxamide,
N-(2-((3-pyridinyl)methyl-1-azabicyclo[2.2.2]oct-3-yl)-6-hydroxynaphthalene-2-carboxamide,
N-(2-((3-pyridinyl)methyl-1-azabicyclo[2.2.2]oct-3-yl)-6-acetoxynaphthalene-2-carboxamide,
N-(2-((3-pyridinyl)methyl)-1-azabicyclo[2.2.2]oct-3-yl)-3-phenylprop-2-enamide,
N-(2-((3-pyridinyl)methyl)-1-azabicyclo[2.2.2]oct-3-yl)-3-(3-fluorophenyl)prop-2-enamide,
N-(2-((3-pyridinyl)methyl)-1-azabicyclo[2.2.2]oct-3-yl)-3-(4-methoxyphenyl)prop-2-enamide,
N-(2-((3-pyridinyl)methyl)-1-azabicyclo[2.2.2]oct-3-yl)-2-methyl-3-phenylprop-2-enamide,
N-(2-((3-pyridinyl)methyl)-1-azabicyclo[2.2.2]oct-3-yl)-3-(2-fluorophenyl)prop-2-enamide,
N-(2-((3-pyridinyl)methyl)-1-azabicyclo[2.2.2]oct-3-yl)-3-(3-methylphenyl)prop-2-enamide,
N-(2-((3-pyridinyl)methyl)-1-azabicyclo[2.2.2]oct-3-yl)-3-(4-fluorophenyl)prop-2-enamide,
N-(2-((3-pyridinyl)methyl)-1-azabicyclo[2.2.2]oct-3-yl)-3-(4-methylphenyl)prop-2-enamide,
N-(2-((3-pyridinyl)methyl)-1-azabicyclo[2.2.2]oct-3-yl)-3-(2-furyl)prop-2-enamide,
N-(2-((3-pyridinyl)methyl)-1-azabicyclo[2.2.2]oct-3-yl)-3-(2-methoxyphenyl)prop-2-enamide,
N-(2-((3-pyridinyl)methyl)-1-azabicyclo[2.2.2]oct-3-yl)-3-(3-bromophenyl)prop-2-enamide,
N-(2-((3-pyridinyl)methyl)-1-azabicyclo[2.2.2]oct-3-yl)-3-(3-methoxyphenyl)prop-2-enamide,
N-(2-((3-pyridinyl)methyl)-1-azabicyclo[2.2.2]oct-3-yl)-3-(3-hydroxyphenyl)prop-2-enamide,
N-(2-((3-pyridinyl)methyl)-1-azabicyclo[2.2.2]oct-3-yl)-3-(4-bromophenyl)prop-2-enamide,
N-(2-((3-pyridinyl)methyl)-1-azabicyclo[2.2.2]oct-3-yl)-3-(4-chlorophenyl)prop-2-enamide,
N-(2-((3-pyridinyl)methyl)-1-azabicyclo[2.2.2]oct-3-yl)-3-(4-hydroxyphenyl)prop-2-enamide,
N-(2-((3-pyridinyl)methyl)-1-azabicyclo[2.2.2]oct-3-yl)-3-(4-hydroxy-3-methoxyphenyl)prop-2-enamide,
N-(2-((3-pyridinyl)methyl)-1-azabicyclo[2.2.2]oct-3-yl)-3-(2-thienyl)prop-2-enamide,
N-(2-((3-pyridinyl)methyl)-1-azabicyclo[2.2.2]oct-3-yl)-3-(3-pyridinyl)prop-2-enamide,
N-(2-((3-pyridinyl)methyl)-1-azabicyclo[2.2.2]oct-3-yl)-3-(4-biphenyl)prop-2-enamide,
N-(2-((3-pyridinyl)methyl)-1-azabicyclo[2.2.2]oct-3-yl)-3-(1-naphthyl)prop-2-enamide,
N-(2-((3-pyridinyl)methyl)-1-azabicyclo[2.2.2]oct-3-yl)-3-(3-thienyl)prop-2-enamide,
N-(2-((3-pyridinyl)methyl)-1-azabicyclo[2.2.2]oct-3-yl)-3-(4-isopropylphenyl)prop-2-enamide,
N-(2-((3-pyridinyl)methyl)-1-azabicyclo[2.2.2]oct-3-yl)-3-methyl-3-phenylprop-2-enamide,
N-(2-((3-pyridinyl)methyl)-1-azabicyclo[2.2.2]oct-3-yl)-3-(3-furyl)prop-2-enamide,
N-(2-((3-pyridinyl)methyl)-1-azabicyclo[2.2.2]oct-3-yl)-2-ethyl-3-phenylprop-2-enamide,
N-(2-((3-pyridinyl)methyl)-1-azabicyclo[2.2.2]oct-3-yl)-3-(2-pyridinyl)prop-2-enamide,
N-(2-((3-pyridinyl)methyl)-1-azabicyclo[2.2.2]oct-3-yl)-3-(3,4-dimethylthieno[2,3-b]thiophen-2-yl)prop-2-enamide,
N-(2-((3-pyridinyl)methyl)-1-azabicyclo[2.2.2]oct-3-yl)-3-(3-methylthien-2-yl)prop-2-enamide,
N-(2-((3-pyridinyl)methyl)-1-azabicyclo[2.2.2]oct-3-yl)-3-(2-naphthyl)prop-2-enamide, and
N-(2-((3-pyridinyl)methyl)-1-azabicyclo[2.2.2]oct-3-yl)-3-(4-methylthiophenyl)prop-2-enamide,
   or a pharmaceutically acceptable salt thereof.

A second genus of alpha7 NNR selective ligands (see U.S. application Ser. No. 11/465,914, Pub. No. 2007 00197579 A1; also see published international application WO 2007/024814 A1; each of which is incorporated herein by reference in its entirety), useful according to the present invention, is represented by Formula 2.

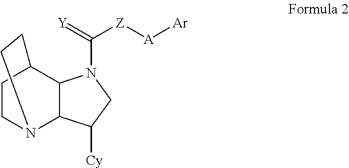

In Formula 2, Y is either oxygen or sulfur, and Z is either nitrogen (i.e., NR′) or a covalent bond. A is either absent or a linker species selected from the group —CR′ R″—, —CR′R″—CR′R″—, —CR′═CR′—, and —C₂—, wherein R′ and R″ are as hereinafter defined. Ar is an aryl group, either carbocyclic or heterocyclic, either monocyclic or fused polycyclic, unsubstituted or substituted; and Cy is a 5- or 6-membered heteroaromatic ring, unsubstituted or substituted. Thus, the invention includes compounds in which Ar is linked to the diazatricycle, at the nitrogen of the depicted pyrrolidine ring, by a carbonyl group-containing functionality, forming an amide or a urea functionality. Ar may be bonded directly to the carbonyl group-containing functionality or may be linked to the carbonyl group-containing functionality through linker A. Furthermore, the invention includes compounds that contain a diazatricycle, containing a 1-azabicyclo[2.2.2]octane. As used in reference to Formula 2, a “carbonyl group-containing functionality” is a moiety of the formula —C(═Y)—Z—, where Y are Z are as defined herein.

In one embodiment, Cy is 3-pyridinyl or 5-pyrimidinyl, Y is oxygen, Z is a covalent bond, and A is absent. In another embodiment, Cy is 3-pyridinyl or 5-pyrimidinyl, Y is oxygen, Z is nitrogen, and A is absent. In a third embodiment, Cy is 3-pyridinyl or 5-pyrimidinyl, Y is oxygen, Z is a covalent bond, and A is a linker species. In a fourth embodiment, Cy is 3-pyridinyl or 5-pyrimidinyl, Y is oxygen, Z is nitrogen, and A is a linker species.

The junction between the azacycle and the azabicycle can be characterized by any of the various relative and absolute stereochemical configurations at the junction sites (e.g., cis or trans, R or S). The compounds have one or more asymmetric carbons and can therefore exist in the form of racemic mixtures, enantiomers and diastereomers. In addition, some of the compounds exist as E and Z isomers about a carbon-carbon double bond. All these individual isomeric compounds and their mixtures are also intended to be within the scope of the present invention.

As used in Formula 2, Ar (“aryl”) includes both carbocyclic and heterocyclic aromatic rings, both monocyclic and fused polycyclic, where the aromatic rings can be 5- or 6-membered rings. Representative monocyclic aryl groups include, but are not limited to, phenyl, furanyl, pyrrolyl, thienyl, pyridinyl, pyrimidinyl, oxazolyl, isoxazolyl, pyrazolyl, imidazolyl, thiazolyl, isothiazolyl and the like. Fused polycyclic aryl groups are those aromatic groups that include a 5- or 6-membered aromatic or heteroaromatic ring as one or more rings in a fused ring system. Representative fused polycyclic aryl groups include naphthalene, anthracene, indolizine, indole, isoindole, benzofuran, benzothiophene, indazole, benzimidazole, benzthiazole, purine, quinoline, isoquinoline, cinnoline, phthalazine, quinazoline, quinoxaline, 1,8-naphthyridine, pteridine, carbazole, acridine, phenazine, phenothiazine, phenoxazine, and azulene.

As used in Formula 2, “Cy” groups are 5- and 6-membered ring heteroaromatic groups. Representative Cy groups include pyridinyl, pyrimidinyl, furanyl, pyrrolyl, thienyl, oxazolyl, isoxazolyl, pyrazolyl, imidazolyl, thiazolyl, isothiazolyl, and the like, where pyridinyl is preferred.

Individually, Ar and Cy can be unsubstituted or can be substituted with 1, 2, or 3 substituents, such as alkyl, alkenyl, heterocyclyl, cycloalkyl, aryl, substituted aryl, arylalkyl, substituted arylalkyl, halo (e.g., F, Cl, Br, or I), —OR′, —NR′R″, —CF₃, —CN, —NO₂, —C₂R′, —SR′, —N₃, —C(═O)NR′R″, —NR′C(═O)R″, —C(═O)R′, —C(═O)OR′, —OC(═O)R′, —O(CR′R″)ᵣC(═O)R′, —O(CR′R″)ᵣNR″C(═O)R′, —O(CR′R″)ᵣNR″SO₂R′, —OC(═O)NR′R″, —NR′C(═O)O R″, —SO₂R′, —SO₂NR′R″, and —NR′SO₂R″, where R′ and R″ are individually hydrogen, C₁-C₅ alkyl (e.g., straight chain or branched alkyl, preferably C₁-C₅, such as methyl, ethyl, or isopropyl), cycloalkyl (e.g., C₃₋₈ cyclic alkyl), heterocyclyl, aryl, or arylalkyl (such as benzyl), and r is an integer from 1 to 6. R′ and R″ can also combine to form a cyclic functionality.

Compounds of Formula 2 form acid addition salts which are useful according to the present invention. Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate. The salts may be in some cases hydrates or ethanol solvates.

Representative compounds of Formula 2 include:
5-benzoyl-3-pyridin-3-yl-1,5-diazatricyclo[5.2.2.0<2,6>]undecane,
5-(2-fluorobenzoyl)-3-pyridin-3-yl-1,5-diazatricyclo[5.2.2.0<2,6>]undecane,
5-(3-fluorobenzoyl)-3-pyridin-3-yl-1,5-diazatricyclo[5.2.2.0<2,6>]undecane,
5-(4-fluorobenzoyl)-3-pyridin-3-yl-1,5-diazatricyclo[5.2.2.0<2,6>]undecane,
5-(2-chlorobenzoyl)-3-pyridin-3-yl-1,5-diazatricyclo[5.2.2.0<2,6>]undecane,
5-(3-chlorobenzoyl)-3-pyridin-3-yl-1,5-diazatricyclo[5.2.2.0<2,6>]undecane,
5-(4-chlorobenzoyl)-3-pyridin-3-yl-1,5-diazatricyclo[5.2.2.0<2,6>]undecane,
5-(2-bromobenzoyl)-3-pyridin-3-yl-1,5-diazatricyclo[5.2.2.0<2,6>]undecane,
5-(3-bromobenzoyl)-3-pyridin-3-yl-1,5-diazatricyclo[5.2.2.0<2,6>]undecane,
5-(4-bromobenzoyl)-3-pyridin-3-yl-1,5-diazatricyclo[5.2.2.0<2,6>]undecane,
5-(2-iodobenzoyl)-3-pyridin-3-yl-1,5-diazatricyclo[5.2.2.0<2,6>]undecane,
5-(3-iodobenzoyl)-3-pyridin-3-yl-1,5-diazatricyclo[5.2.2.0<2,6>]undecane,
5-(4-iodobenzoyl)-3-pyridin-3-yl-1,5-diazatricyclo[5.2.2.0<2,6>]undecane,
5-(2-methylbenzoyl)-3-pyridin-3-yl-1,5-diazatricyclo[5.2.2.0<2,6>]undecane,
5-(3-methylbenzoyl)-3-pyridin-3-yl-1,5-diazatricyclo[5.2.2.0<2,6>]undecane,
5-(4-methylbenzoyl)-3-pyridin-3-yl-1,5-diazatricyclo[5.2.2.0<2,6>]undecane,
5-(2-methoxybenzoyl)-3-pyridin-3-yl-1,5-diazatricyclo[5.2.2.0<2,6>]undecane,
5-(3-methoxybenzoyl)-3-pyridin-3-yl-1,5-diazatricyclo[5.2.2.0<2,6>]undecane,
5-(4-methoxybenzoyl)-3-pyridin-3-yl-1,5-diazatricyclo[5.2.2.0<2,6>]undecane,
5-(2-methylthiobenzoyl)-3-pyridin-3-yl-1,5-diazatricyclo[5.2.2.0<2,6>]undecane,
5-(3-methylthiobenzoyl)-3-pyridin-3-yl-1,5-diazatricyclo[5.2.2.0<2,6>]undecane,
5-(4-methylthiobenzoyl)-3-pyridin-3-yl-1,5-diazatricyclo[5.2.2.0<2,6>]undecane,
5-(2-phenylbenzoyl)-3-pyridin-3-yl-1,5-diazatricyclo[5.2.2.0<2,6>]undecane,
5-(3-phenylbenzoyl)-3-pyridin-3-yl-1,5-diazatricyclo[5.2.2.0<2,6>]undecane,
5-(4-phenylbenzoyl)-3-pyridin-3-yl-1,5-diazatricyclo[5.2.2.0<2,6>]undecane,
5-(2-phenoxybenzoyl)-3-pyridin-3-yl-1,5-diazatricyclo[5.2.2.0<2,6>]undecane,
5-(3-phenoxybenzoyl)-3-pyridin-3-yl-1,5-diazatricyclo[5.2.2.0<2,6>]undecane,
5-(4-phenoxybenzoyl)-3-pyridin-3-yl-1,5-diazatricyclo[5.2.2.0<2,6>]undecane,
5-(2-phenylthiobenzoyl)-3-pyridin-3-yl-1,5-diazatricyclo[5.2.2.0<2,6>]undecane,
5-(3-phenylthiobenzoyl)-3-pyridin-3-yl-1,5-diazatricyclo[5.2.2.0<2,6>]undecane,
5-(4-phenylthiobenzoyl)-3-pyridin-3-yl-1,5-diazatricyclo[5.2.2.0<2,6>]undecane,
5-(2-cyanobenzoyl)-3-pyridin-3-yl-1,5-diazatricyclo[5.2.2.0<2,6>]undecane,
5-(3-cyanobenzoyl)-3-pyridin-3-yl-1,5-diazatricyclo[5.2.2.0<2,6>]undecane,
5-(4-cyanobenzoyl)-3-pyridin-3-yl-1,5-diazatricyclo[5.2.2.0<2,6>]undecane,
5-(2-trifluoromethylbenzoyl)-3-pyridin-3-yl-1,5-diazatricyclo[5.2.2.0<2,6>]undecane,
5-(3-trifluoromethylbenzoyl)-3-pyridin-3-yl-1,5-diazatricyclo[5.2.2.0<2,6>]undecane,
5-(4-trifluoromethylbenzoyl)-3-pyridin-3-yl-1,5-diazatricyclo[5.2.2.0<2,6>]undecane,
5-(2-dimethylaminobenzoyl)-3-pyridin-3-yl-1,5-diazatricyclo[5.2.2.0<2,6>]undecane,
5-(3-dimethylaminobenzoyl)-3-pyridin-3-yl-1,5-diazatricyclo[5.2.2.0<2,6>]undecane,
5-(4-dimethylaminobenzoyl)-3-pyridin-3-yl-1,5-diazatricyclo[5.2.2.0<2,6>]undecane,
5-(2-ethynylbenzoyl)-3-pyridin-3-yl-1,5-diazatricyclo[5.2.2.0<2,6>]undecane,
5-(3-ethynylbenzoyl)-3-pyridin-3-yl-1,5-diazatricyclo[5.2.2.0<2,6>]undecane,
5-(4-ethynylbenzoyl)-3-pyridin-3-yl-1,5-diazatricyclo[5.2.2.0<2,6>]undecane,
5-(3,4-dichlorobenzoyl)-3-pyridin-3-yl-1,5-diazatricyclo[5.2.2.0<2,6>]undecane,
5-(2,4-dimethoxybenzoyl)-3-pyridin-3-yl-1,5-diazatricyclo[5.2.2.0<2,6>]undecane,
5-(3,4,5-trimethoxybenzoyl)-3-pyridin-3-yl-1,5-diazatricyclo[5.2.2.0<2,6>]undecane,
5-(naphth-1-ylcarbonyl)-3-pyridin-3-yl-1,5-diazatricyclo[5.2.2.0<2,6>]undecane,
5-(naphth-2-ylcarbonyl)-3-pyridin-3-yl-1,5-diazatricyclo[5.2.2.0<2,6>]undecane,
5-(thien-2-ylcarbonyl)-3-pyridin-3-yl-1,5-diazatricyclo[5.2.2.0<2,6>]undecane,
5-(thien-3-ylcarbonyl)-3-pyridin-3-yl-1,5-diazatricyclo[5.2.2.0<2,6>]undecane,
5-(furan-2-ylcarbonyl)-3-pyridin-3-yl-1,5-diazatricyclo[5.2.2.0<2,6>]undecane,
5-(benzothien-2-ylcarbonyl)-3-pyridin-3-yl-1,5-diazatricyclo[5.2.2.0<2,6>]undecane,
5-(benzofuran-2-ylcarbonyl)-3-pyridin-3-yl-1,5-diazatricyclo[5.2.2.0<2,6>]undecane,
5-(7-methoxybenzofuran-2-ylcarbonyl)-3-pyridin-3-yl-1,5-diazatricyclo[5.2.2.0<2,6>]undecane, and
5-(1H-indol-3-ylcarbonyl)-3-pyridin-3-yl-1,5-diazatricyclo[5.2.2.0<2,6>]undecane,
   or a pharmaceutically acceptable salt thereof.

Other compounds representative of Formula 2 include:
5-(phenylacetyl)-3-pyridin-3-yl-1,5-diazatricyclo[5.2.2.0<2,6>]undecane,
5-(diphenylacetyl)-3-pyridin-3-yl-1,5-diazatricyclo[5.2.2.0<2,6>]undecane,
5-(2-phenylpropanoyl)-3-pyridin-3-yl-1,5-diazatricyclo[5.2.2.0<2,6>]undecane, and
5-(3-phenylprop-2-enoyl)-3-pyridin-3-yl-1,5-diazatricyclo[5.2.2.0<2,6>]undecane,
   or a pharmaceutically acceptable salt thereof.

Other compounds representative of Formula 2 include:
5-N-phenylcarbamoyl-3-pyridin-3-yl-1,5-diazatricyclo[5.2.2.0<2,6>]undecane,
5-(N-(2-fluorophenyl)carbamoyl)-3-pyridin-3-yl-1,5-diazatricyclo[5.2.2.0<2,6>]undecane,
5-(N-(3-fluorophenyl)carbamoyl)-3-pyridin-3-yl-1,5-diazatricyclo[5.2.2.0<2,6>]undecane,
5-(N-(4-fluorophenyl)carbamoyl)-3-pyridin-3-yl-1,5-diazatricyclo[5.2.2.0<2,6>]undecane,
5-(N-(2-chlorophenyl)carbamoyl)-3-pyridin-3-yl-1,5-diazatricyclo[5.2.2.0<2,6>]undecane,
5-(N-(3-chlorophenyl)carbamoyl)-3-pyridin-3-yl-1,5-diazatricyclo[5.2.2.0<2,6>]undecane,
5-(N-(4-chlorophenyl)carbamoyl)-3-pyridin-3-yl-1,5-diazatricyclo[5.2.2.0<2,6>]undecane,
5-(N-(2-bromophenyl)carbamoyl)-3-pyridin-3-yl-1,5-diazatricyclo[5.2.2.0<2,6>]undecane,
5-(N-(3-bromophenyl)carbamoyl)-3-pyridin-3-yl-1,5-diazatricyclo[5.2.2.0<2,6>]undecane,
5-(N-(4-bromophenyl)carbamoyl)-3-pyridin-3-yl-1,5-diazatricyclo[5.2.2.0<2,6>]undecane,
5-(N-(2-iodophenyl)carbamoyl)-3-pyridin-3-yl-1,5-diazatricyclo[5.2.2.0<2,6>]undecane,
5-(N-(3-iodophenyl)carbamoyl)-3-pyridin-3-yl-1,5-diazatricyclo[5.2.2.0<2,6>]undecane,
5-(N-(4-iodophenyl)carbamoyl)-3-pyridin-3-yl-1,5-diazatricyclo[5.2.2.0<2,6>]undecane,
5-(N-(2-methylphenyl)carbamoyl)-3-pyridin-3-yl-1,5-diazatricyclo[5.2.2.0<2,6>]undecane,
5-(N-(3-methylphenyl)carbamoyl)-3-pyridin-3-yl-1,5-diazatricyclo[5.2.2.0<2,6>]undecane,
5-(N-(4-methylphenyl)carbamoyl)-3-pyridin-3-yl-1,5-diazatricyclo[5.2.2.0<2,6>]undecane,
5-(N-(2-methoxyphenyl)carbamoyl)-3-pyridin-3-O-1,5-diazatricyclo[5.2.2.0<2,6>]undecane,
5-(N-(3-methoxyphenyl)carbamoyl)-3-pyridin-3-yl-1,5-diazatricyclo[5.2.2.0<2,6>]undecane,
5-(N-(4-methoxyphenyl)carbamoyl)-3-pyridin-3-yl-1,5-diazatricyclo[5.2.2.0<2,6>]undecane,
5-(N-(2-methylthiophenyl)carbamoyl)-3-pyridin-3-O-1,5-diazatricyclo[5.2.2.0<2,6>]undecane,
5-(N-(3-methylthiophenyl)carbamoyl)-3-pyridin-3-yl-1,5-diazatricyclo[5.2.2.0<2,6>]undecane,
5-(N-(4-methylthiophenyl)carbamoyl)-3-pyridin-3-yl-1,5-diazatricyclo[5.2.2.0<2,6>]undecane,
5-(N-(2-phenylphenyl)carbamoyl)-3-pyridin-3-yl-1,5-diazatricyclo[5.2.2.0<2,6>]undecane,
5-(N-(3-phenylphenyl)carbamoyl)-3-pyridin-3-yl-1,5-diazatricyclo[5.2.2.0<2,6>]undecane,
5-(N-(4-phenylphenyl)carbamoyl)-3-pyridin-3-yl-1,5-diazatricyclo[5.2.2.0<2,6>]undecane,
5-(N-(2-phenoxyphenyl)carbamoyl)-3-pyridin-3-yl-1,5-diazatricyclo[5.2.2.0<2,6>]undecane,
5-(N-(3-phenoxyphenyl)carbamoyl)-3-pyridin-3-yl-1,5-diazatricyclo[5.2.2.0<2,6>]undecane,
5-(N-(4-phenoxyphenyl)carbamoyl)-3-pyridin-3-yl-1,5-diazatricyclo[5.2.2.0<2,6>]undecane,
5-(N-(2-phenylthiophenyl)carbamoyl)-3-pyridin-3-yl-1,5-diazatricyclo[5.2.2.0<2,6>]undecane,
5-(N-(3-phenylthiophenyl)carbamoyl)-3-pyridin-3-yl-1,5-diazatricyclo[5.2.2.0<2,6>]undecane,
5-(N-(4-phenylthiophenyl)carbamoyl)-3-pyridin-3-yl-1,5-diazatricyclo[5.2.2.0<2,6>]undecane,
5-(N-(2-cyanophenyl)carbamoyl)-3-pyridin-3-yl-1,5-diazatricyclo[5.2.2.0<2,6>]undecane,
5-(N-(3-cyanophenyl)carbamoyl)-3-pyridin-3-yl-1,5-diazatricyclo[5.2.2.0<2,6>]undecane,
5-(N-(4-cyanophenyl)carbamoyl)-3-pyridin-3-yl-1,5-diazatricyclo[5.2.2.0<2,6>]undecane,
5-(N-(2-trifluoromethylphenyl)carbamoyl)-3-pyridin-3-yl-1,5-diazatricyclo[5.2.2.0<2,6>]undecane,
5-(N-(3-trifluoromethylphenyl)carbamoyl)-3-pyridin-3-yl-1,5-diazatricyclo[5.2.2.0<2,6>]undecane,
5-(N-(4-trifluoromethylphenyl)carbamoyl)-3-pyridin-3-yl-1,5-diazatricyclo[5.2.2.0<2,6>]undecane,
5-(N-(2-dimethylaminophenyl)carbamoyl)-3-pyridin-3-yl-1,5-diazatricyclo[5.2.2.0<2,6>]undecane,
5-(N-(3-dimethylaminophenyl)carbamoyl)-3-pyridin-3-yl-1,5-diazatricyclo[5.2.2.0<2,6>]undecane,
5-(N-(4-dimethylaminophenyl)carbamoyl)-3-pyridin-3-yl-1,5-diazatricyclo[5.2.2.0<2,6>]undecane,
5-(N-(2-ethynylphenyl)carbamoyl)-3-pyridin-3-yl-1,5-diazatricyclo[5.2.2.0<2,6>]undecane,
5-(N-(3-ethynylphenyl)carbamoyl)-3-pyridin-3-yl-1,5-diazatricyclo[5.2.2.0<2,6>]undecane,
5-(N-(4-ethynylphenyl)carbamoyl)-3-pyridin-3-yl-1,5-diazatricyclo[5.2.2.0<2,6>]undecane,
5-(N-(3,4-dichlorophenyl)carbamoyl)-3-pyridin-3-yl-1,5-diazatricyclo[5.2.2.0<2,6>]undecane,
5-(N-(2,4-dimethoxyphenyl)carbamoyl)-3-pyridin-3-yl-1,5-diazatricyclo[5.2.2.0<2,6>]undecane,
5-(N-(3,4,5-trimethoxyphenyl)carbamoyl)-3-pyridin-3-yl-1,5-diazatricyclo[5.2.2.0<2,6>]undecane,
5-(N-(1-naphthyl)carbamoyl)-3-pyridin-3-yl-1,5-diazatricyclo[5.2.2.0<2,6>]undecane, and
5-(N-(2-naphthyl)carbamoyl)-3-pyridin-3-yl-1,5-diazatricyclo[5.2.2.0<2,6>]undecane.
   Other compounds representative of Formula 2 include:
5-(N-benzylcarbamoyl)-3-pyridin-3-yl-1,5-diazatricyclo[5.2.2.0<2,6>]undecane,
5-(N-(4-bromobenzyl)carbamoyl)-3-pyridin-3-yl-1,5-diazatricyclo[5.2.2.0<2,6>]undecane,
5-(N-(4-methoxybenzyl)carbamoyl)-3-pyridin-3-yl-1,5-diazatricyclo[5.2.2.0<2,6>]undecane,
5-(N-(1-phenylethyl)carbamoyl)-3-pyridin-3-yl-1,5-diazatricyclo[5.2.2.0<2,6>]undecane, and
5-(N-(diphenylmethyl)carbamoyl)-3-pyridin-3-yl-1,5-diazatricyclo[5.2.2.0<2,6>]undecane,
   or a pharmaceutically acceptable salt thereof.

In each of these compounds, a 3-pyridin-3-yl-1,5-diazatricyclo[5.2.2.0<2,6>]undecane moiety has the structure, with a partial numbering scheme provided, shown below:

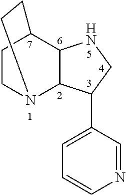

The nitrogen at the position indicated above as the 5-position is the nitrogen involved in the formation of the amides, thioamides, ureas and thioureas described herein.

Compounds useful according to the present invention also include compounds of Formula 3:

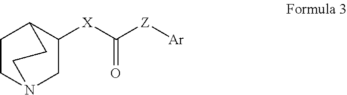

In Formula 3, X is either oxygen or nitrogen (i.e., NR′), and Z is either nitrogen (i.e., NR′), —CR′═CR′— or a covalent bond, provided that X must be nitrogen when Z is —CR′═CR′— or a covalent bond, and further provided that X and Z are not simultaneously nitrogen. Ar is an aryl group, either carbocyclic or heterocyclic, either monocyclic or fused polycyclic, unsubstituted or substituted; R′ is hydrogen, C₁-C₈, alkyl (e.g., straight chain or branched alkyl, preferably C₁-C₅, such as methyl, ethyl, or isopropyl), aryl, or arylalkyl (such as benzyl).

Compounds in which X is oxygen and Z is nitrogen are disclosed as alpha7 selective ligands in, for instance, PCT WO 97/30998 and U.S. Pat. No. 6,054,464, each of which is incorporated herein in its entirety.

Compounds in which X is nitrogen and Z is covalent bond are disclosed as alpha7 selective ligands in, for instance, PCTs WO 02/16355, WO 02/16356, WO 02/16358, WO 04/029050, WO 04/039366, WO 04/052461, WO 07/038,367, and in U.S. Pat. No. 6,486,172, U.S. Pat. No. 6,500,840, U.S. Pat. No. 6,599,916, U.S. Pat. No. 7,001,914, U.S. Pat. No. 7,067,515, and U.S. Pat. No. 7,176,198, each of which is herein incorporated herein in its entirety.

Compounds in which X is nitrogen and Z is —CR′═CR′— are disclosed as alpha7 selective ligands in, for instance, PCT WO 01/036417 and U.S. Pat. No. 6,683,090, each of which is incorporated herein in its entirety.

Particular embodiments according to the general Formula 3 include the following:
N-((3R)-1-azabicyclo[2.2.2]oct-3-yl)-5-phenylthiophene-2-carboxamide;
N-((3R)-1-azabicyclo[2.2.2]oct-3-yl)-2-phenyl-1,3-thiazole-5-carboxamide;
N-((3R)-1-azabicyclo[2.2.2]oct-3-yl)-5-phenyl-1,3-oxazole-2-carboxamide;
N-((3R)-1-azabicyclo[2.2.2]oct-3-yl)-5-phenyl-1,3,4-oxadiazole-2-carboxamide;
N-[(3R)-1-azabicyclo[2.2.2]oct-3-y-l]-4-(4-hydroxyphenoxy)benzamide;
N-[(3R)-1-azabicyclo[2.2.2]oct-3-yl]-4-(-4-acetamidophenoxy)benzamide;
N-[(3R)-1-azabicyclo[2.2.2]oct-3-yl]-4-phenoxybenzamide;
N-[(3R)-1-azabicyclo[2.2.2]oct-3-yl]-4-benzylbenzamide;
N-[(3R)-1-azabicyclo[2.2.2]oct-3-yl]-4-(phenylsulfanyl)benzamide;
N-[(3R)-1-azabicyclo[2.2.2]oct-3-yl]-3-phenoxybenzamide;
N-[(3R)-1-azabicyclo[2.2.2]oct-3-yl]-4-benzoylbenzamide;
N-[(3R)-1-azabicyclo[2.2.2]oct-3-yl]-4-(4-fluorophenoxy)benzamide;
N-[(3R)-1-azabicyclo[2.2.2]oct-3-yl]-4-(2-fluorophenoxy)benzamide;
N-[(3R)-1-azabicyclo[2.2.2]oct-3-yl]-4-(3-fluorophenoxy)benzamide;
N-[(3R)-1-azabicyclo[2.2.2]oct-3-yl]-4-(2-chlorophenoxy)benzamide;
N-[(3R)-1-azabicyclo[2.2.2]oct-3-yl]-4-(3-chlorophenoxy)benzamide;
N-[(3R)-1-azabicyclo[2.2.2]oct-3-yl]-4-(4-chlorophenoxy)benzamide;
N-[(3R)-1-azabicyclo[2.2.2]oct-3-yl]-4-(2-methoxyphenoxy)benzamide;
N-[(3R)-1-azabicyclo[2.2.2]oct-3-yl]-4-(3-methoxyphenoxy)benzamide;
N-[(3R)-1-azabicyclo[2.2.2]oct-3-yl]-4-(4-methoxyphenoxy)benzamide;
N-[(3R)-1-azabicyclo[2.2.2]oct-3-yl]-4-(3-chlorophenylsulfanyl)benzamide;
N-[(3R)-1-azabicyclo[2.2.2]oct-3-yl]-4-(4-methoxyphenoxy)benzamide;
N-[(3R)-1-azabicyclo[2.2.2]oct-3-yl]-4-(3-chlorophenylsulfanyl)benzamide;
N-[(3R)-1-azabicyclo[2.2.2]oct-3-yl]-4-(4-chlorophenylsulfanyl)benzamide;
N-[(3R)-1-azabicyclo[2.2.2]oct-3-yl]-4-(3-methoxyphenylsulfanyl)-benzamide;
N-[(3R)-1-azabicyclo[2.2.2]oct-3-yl]-4(2-methoxyphenylsulfanyl)-benzamide;
N-(2-methyl-1-azabicyclo[2.2.2]oct-3-yl)-4-phenoxybenzamide;
N-((3R)-1-azabicyclo[2.2.2]oct-3-yl)-4-(pyridin-3-yloxy)benzamide;
N-phenylcarbamic acid 1-azabicyclo[2.2.2]octan-3-yl ester;
N-(4-bromophenyl)carbamic acid 1-azabicyclo[2.2.2]octan-3-yl ester;
N-(4-methylphenyl)carbamic acid 1-azabicyclo[2.2.2]octan-3-yl ester;
N-(4-methoxyphenyl)carbamic acid 1-azabicyclo[2.2.2]octan-3-yl ester;
N-(3,4-dichlorophenyl)carbamic acid 1-azabicyclo[2.2.2]octan-3-yl ester;
N-(4-cyanophenyl)carbamic acid 1-azabicyclo[2.2.2]octan-3-yl ester;
N-phenylcarbamic acid 1-azabicyclo[2.2.1]heptan-3-yl ester;
N-(3-methoxyphenyl)carbamic acid 1-azabicyclo[2.2.2]octan-3-yl ester;
N-phenylthiocarbamic acid 1-azabicyclo[2.2.2]octan-3-yl ester;
N-(2-pyridyl)carbamic acid 1-azabicyclo[2.2.2]octan-3-yl ester;
N-(1-naphthyl)carbamic acid 1-azabicyclo[2.2.2]octan-3-yl ester;
N-phenylcarbamic acid (3R)-1-azabicyclo[2.2.2]octan-3-yl ester;
N-phenylcarbamic acid (3S)-1-azabicyclo[2.2.2]octan-3-yl ester;
N-(4-pyridyl)carbamic acid 1-azabicyclo[2.2.2]octan-3-yl ester;
N-(m-biphenyl)carbamic acid 1-azabicyclo[2.2.2]octan-3-yl ester;
N-(3-quinolinyl)carbamic acid 1-azabicyclo[2.2.2]octan-3-yl ester;
N-(1-azabicyclo[2.2.2]oct-3-yl)(E-3-phenylpropenamide); and
N-(1-azabicyclo[2.2.2]oct-3-yl)(3-phenylpropenamide);
   or a pharmaceutically acceptable salt thereof.

Compounds useful according to the present invention also include compounds of Formula 4:

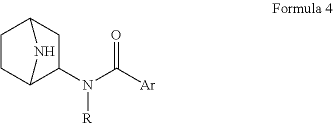

In Formula 4, Ar is an aryl group, either carbocyclic or heterocyclic, either monocyclic or fused polycyclic, unsubstituted or substituted; R is hydrogen, C₁-C₈ alkyl (e.g., straight chain or branched alkyl, preferably C₁-C₅, such as methyl, ethyl, or isopropyl), aryl, or arylalkyl (such as benzyl).

Such compounds are disclosed as alpha7 selective ligands in, for instance, PCTs WO 03/018585, WO 03/018586, WO 03/022856, WO 03/070732, WO 03/072578, WO 04/039815, and WO 04/052348, and U.S. Pat. No. 6,562,816, each of which is incorporated herein in its entirety.

Particular embodiments according to the general Formula 4 include the following:
N-(7-azabicyclo[2.2.1]hept-2-yl)-5-phenylthiophene-2-carbozamide;
N-(7-azabicyclo[2.2.1]hept-2-yl)-5-(2-pyridinyl)thiophene-2-carbozamide; and
N-(7-azabicyclo[2.2.1]hept-2-yl)-5-phenylfuran-2-carbozamide;
   or a pharmaceutically acceptable salt thereof.

Compounds useful according to the present invention also include compounds of Formula 5:

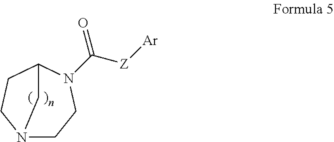

In Formula 5, n is 1 or 2; Ar is an aryl group, either carbocyclic or heterocyclic, either monocyclic or fused polycyclic, unsubstituted or substituted; and Z is oxygen, —C≡C—, —CH═CH—, or a covalent bond.

Such compounds are disclosed as alpha7 selective ligands in, for instance, PCTs WO 00/058311, WO 04/016616, WO 04/016617, 04/061510, WO 04/061511 and WO 04/076453, each of which is incorporated herein in its entirety.

Particular embodiments according to the general Formula 5 include the following:
(1,4-diazabicyclo[3.2.2]non-4-yl)(4-methoxyphenyl)methanone;
(1,4-diazabicyclo[3.2.2]non-4-yl)(5-chlorofuran-2-yl)methanone;
(1,4-diazabicyclo[3.2.2]non-4-yl)(5-bromothiophen-2-yl)methanone;
(1,4-diazabicyclo[3.2.2]non-4-yl)(4-phenoxyphenyl)methanone;
(1,4-diaza bicyclo[3.2.2]non-4-yl)(5-phenylfuran-2-yl)methanone;
(1,4-diazabicyclo[3.2.2]non-4-yl)(5-(3-pyridinyl)thiophen-2-yl)methanone; and
1-(1,4-diazabicyclo[3.2.2]non-4-yl)-3-phenylpropenone;
   or a pharmaceutically acceptable salt thereof.

Compounds useful according to the present invention also include compounds of Formula 6:

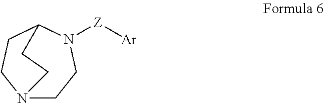

In Formula 6, Ar is a fused polycyclic, heterocyclic aryl group, unsubstituted or substituted; and Z is —CH₂— or a covalent bond.

Such compounds are disclosed as alpha7 selective ligands in, for instance, PCTs WO 03/119837 and WO 05/111038 and U.S. Pat. No. 6,881,734, each of which is herein incorporated by reference in its entirety.

Particular embodiments according to the general Formula 6 include the following:
4-benzoxazol-2-yl-1,4-diazabicyclo[3.2.2]nonane;
4-benzothiazol-2-yl-1,4-diazabicyclo[3.2.2]nonane;
4-benzoxazol-2-yl-1,4-diazabicyclo[3.2.2.]nonane;
4-oxazolo[5,4-b]pyridine-2-yl-1,4-diazabicyclo[3.2.2.]nonane; and
4-(1H-benzimidazol-2-yl-1,4-diazabicyclo[3.2.2]nonane;
   or a pharmaceutically acceptable salt thereof.

Compounds useful according to the present invention also include compounds of Formula 7:

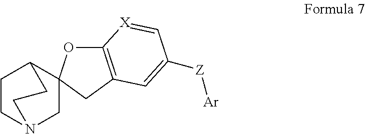

In Formula 7, Ar is an aryl group, either carbocyclic or heterocyclic, either monocyclic or fused polycyclic, unsubstituted or substituted; X is either CH or N; Z is either oxygen, nitrogen (NR) or a covalent bond; and R is H or alkyl. Optionally, “Z—Ar” is absent from Formula 7.

Such compounds are disclosed as alpha7 selective ligands in, for instance, PCTs WO 00/042044, WO 02/096912, WO 03/087102, WO 03/087103, WO 03/087104, WO 05/030778, WO 05/042538 and WO 05/066168, and U.S. Pat. No. 6,110,914, U.S. Pat. No. 6,369,224, U.S. Pat. No. 6,569,865, U.S. Pat. No. 6,703,502, U.S. Pat. No. 6,706,878, U.S. Pat. No. 6,995,167, U.S. Pat. No. 7,186,836 and U.S. Pat. No. 7,196,096, each of which is incorporated herein by reference in its entirety.

Particular embodiments according to the general Formula 7 include the following:
spiro[1-azabicyclo[2.2.2]octane-3,2′-(3′H)-furo[2,3-b]pyridine];
5′-phenylspiro[1-azabicyclo[2.2.2]octane-3,2′-(3′H)-furo[2,3-b]pyridine];
5′-(3-furanyl)spiro[1-azabicyclo[2.2.2]octane-3,2′-(3′H)-furo[2,3-b]pyridine];
5′-(2-thienyl)spiro[1-azabicyclo[2.2.2]octane-3,2′-(3′H)-furo[2,3-b]pyridine];
5′-(N-phenyl-N-methylamino)spiro[1-azabicyclo[2.2.2]octane-3,2′-(3′H)-furo[2,3-b]pyridine];
5′-(N-3-pyridinyl-N-methylamino)spiro[1-azabicyclo[2.2.2]octane-3,2′-(3′H)-furo[2,3-b]pyridine];
5′-(2-benzofuranyl)spiro[1-azabicyclo[2.2.2]octane-3,2′-(3′H)-furo[2,3-b]pyridine];
5′-(2-benzothiazolyl)spiro[1-azabicyclo[2.2.2]octane-3,2′-(3′H)-furo[2,3-b]pyridine]; and
5′-(3-pyridinyl)spiro[1-azabicyclo[2.2.2]octane-3,2′-(3′H)-furo[2,3-b]pyridine];
   or a pharmaceutically acceptable salt thereof.

Compounds useful according to the present invention also include compounds of Formula 8:

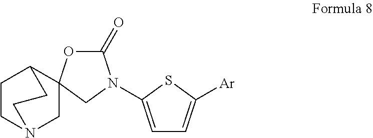

In Formula 8, Ar is an aryl group, either carbocyclic or heterocyclic, either unsubstituted or substituted.

Such compounds are disclosed as alpha7 selective ligands in, for instance, PCTs WO 05/005435 and WO 06/065209, each of which is herein incorporated by reference in its entirety.

Particular embodiments according to the general Formula 8 include the following:
3′-(5-phenylthiophen-2-yl)spiro[1-azabicyclo[2.2.2]octan-3,5′-oxazolidin]-2′-one; and
3′-(5-(3-pyridinyl)thiophen-2-yl)spiro[1-azabicyclo[2.2.2]octan-3,5′-oxazolidin]-2′-one;
   or a pharmaceutically acceptable salt thereof.

Compounds useful according to the present invention also include compounds of Formula 9:

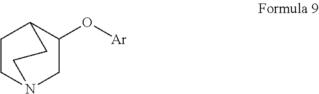

In Formula 9, Ar is an aryl group, either carbocyclic or heterocyclic, either monocyclic or fused polycyclic, unsubstituted or substituted (preferably by aryl or aryloxy substituents).

Such compounds are disclosed as alpha7 selective ligands in, for instance, PCTs WO 04/016608, WO 05/066166, WO 05/066167, WO 07/018,738, and U.S. Pat. No. 7,160,876, each of which is herein incorporated by reference in its entirety.

Particular embodiments according to the general Formula 9 include the following:
2-[4-(1-azabicyclo[2.2.2]oct-3-yloxy)phenyl]-1H-indole;
3-[4-(1-azabicyclo[2.2.2]oct-3-yloxy)phenyl]-1H-indole;
4-[4-(1-azabicyclo[2.2.2]oct-3-yloxy)phenyl]-1H-indole;
5-[4-(1-azabicyclo[2.2.2]oct-3-yloxy)phenyl]-1H-indole;
6-[4-(1-azabicyclo[2.2.2]oct-3-yloxy)phenyl]-1H-indole;
5-[6-(1-azabicyclo[2.2.2]oct-3-yloxy)pyridazin-3-yl]-1H-indole;
4-[6-(1-azabicyclo[2.2.2]oct-3-yloxy)pyridazin-3-yl]-1H-indole;
5-[4-(1-azabicyclo[2.2.2]oct-3-yloxy)phenyl]-3-methyl-1H-indazole;
5-[2-(1-azabicyclo[2.2.2]oct-3-yloxy)pyrimidin-5-yl]-1H-indole; and
6-[4-(1-azabicyclo[2.2.2]oct-3-yloxy)phenyl]-1,3-benzothiazo-3-amine;
   or a pharmaceutically acceptable salt thereof.

Compounds useful according to the present invention also include compounds of Formula 10:

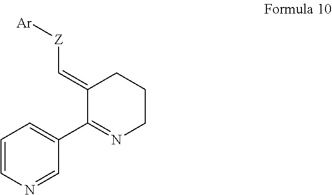

In Formula 10, Ar is an phenyl group, unsubstituted or substituted, and Z is either —CH═CH— or a covalent bond.

Such compounds are disclosed as alpha7 ligands in, for instance, PCTs WO 92/15306, WO 94/05288, WO 99/10338, WO04/019943, WO 04/052365 and WO 06/133303, and U.S. Pat. No. 5,741,802 and U.S. Pat. No. 5,977,144, each of which is herein incorporated by reference in its entirety.

Particular embodiments according to the general Formula 10 include the following:
3-(2,4-dimethoxybenzylidene)anabaseine;
3-(4-hydroxybenzylidene)anabaseine;
3-(4-methoxybenzylidene)anabaseine;
3-(4-aminobenzylidene)anabaseine;
3-(4-hydroxy-2-methoxybenzylidene)anabaseine;
3-(2-hydroxy-4-methoxybenzylidene)anabaseine;
3-(4-isopropoxybenzylidene)anabaseine;
3-(4-acetylaminocinnamylidene)anabaseine;
3-(4-hydroxycinnamylidene)anabaseine;
3-(4-methoxycinnamylidene)anabaseine;
3-(4-hydroxy-2-methoxycinnamylidene)anabaseine;
3-(2,4-dimethoxycinnamylidene)anabaseine; and
3-(4-acetoxycinnamylidene)anabaseine;
   or a pharmaceutically acceptable salt thereof.

Compounds useful according to the present invention also include compounds of Formula 11:

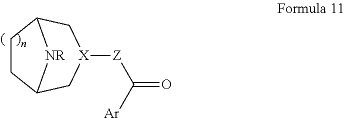

In Formula 11, n is 1 or 2; R is H or alkyl, but most preferably methyl; X is nitrogen or CH; Z is NH or a covalent bond, and when X is nitrogen, Z must be a covalent bond; and Ar is an indolyl, indazolyl, 1,2-benzisoxazolyl or 1,2-benzisothiazolyl moiety, attached in each case via the 3 position to the depicted carbonyl.

Such compounds are disclosed as alpha7 ligands in, for instance, PCT WO 06/001894, herein incorporated by reference in its entirety.

Particular embodiments according to the general Formula 11 include the following:
(8-methyl-8-azabicyclo[3.2.1]oct-3-yl)-6-(2-thienyl)-7H-indazole-3-carboxamide;
3-((3-methyl-3,8-diazabicyclo[3.2.1]oct-8-yl)carbonyl)-7H-indazole;
3-((8-methyl-3,8-diazabicyclo[3.2.1]oct-3-yl)carbonyl)-7H-indazole;
5-methoxy-N-(9-methyl-9-azabicyclo[3.2.1]non-3-yl)-7H-indazole-3-carboxamide; and
6-methoxy-N-(9-methyl-9-azabicyclo[3.2.1]non-3-yl)-1,2-benzisothiazole-3-carboxamide;
   or a pharmaceutically acceptable salt thereof.

As will be appreciated by those skilled in the art the compounds provided may be formulated as pharmaceutical compositions to incorporate a compound of the present invention which, when employed in effective amounts, interacts with relevant nicotinic receptor sites of a subject, and acts as a therapeutic agent to treat and prevent a wide variety of conditions and disorders. The pharmaceutical compositions provide therapeutic benefit to individuals suffering from affected disorders or exhibiting clinical manifestations of affected disorders, in that the compounds within those compositions, when employed in effective amounts, are believed to: (i) exhibit nicotinic pharmacology and affect relevant nicotinic receptors sites, for example by acting as a pharmacological agonist to activate a nicotinic receptor; or (ii) elicit neurotransmitter secretion, and hence prevent and suppress the symptoms associated with those diseases; or both.

The present invention further provides pharmaceutical compositions that include effective amounts of compounds of the formulae of the present invention and salts and solvates, thereof, and one or more pharmaceutically acceptable carriers, diluents, or excipients. The compounds of the formulae of the present invention, including salts and solvates, thereof, are as herein described. The carrier(s), diluent(s), or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the formulae of the present invention, including a salt, solvate, or prodrug thereof, with one or more pharmaceutically acceptable carriers, diluents or excipients.

### Synthetic Examples

### (2S,3R)-3-amino-2-((3-pyridinyl)methyl)-1-azabicyclo[2.2.2]octane di-p-toluoyl-D-tartrate salt

The following large scale synthesis of (2S,3R)-3-amino-2-((3-pyridinyl)methyl)-1-azabicyclo[2.2.2]octane di-p-toluoyl-D-tartrate salt is representative.

### 2-((3-Pyridinyl)methylene)-1-azabicyclo[2.2.2]octan-3-one

3-Quinuclidinone hydrochloride (8.25 kg, 51.0 mol) and methanol (49.5 L) were added to a 100 L glass reaction flask, under an nitrogen atmosphere, equipped with a mechanical stirrer, temperature probe, and condenser. Potassium hydroxide (5.55 kg, 99.0 mol) was added via a powder funnel over an approximately 30 min period, resulting in a rise in reaction temperature from 50° C. to 56° C. Over an approximately 2 h period, 3-pyridinecarboxaldehyde (4.80 kg, 44.9 mol) was added to the reaction mixture. The resulting mixture was stirred at 20° C.±5° C. for a minimum of 12 h, as the reaction was monitored by thin layer chromatography (TLC). Upon completion of the reaction, the reaction mixture was filtered through a sintered glass funnel and the filter cake was washed with methanol (74.2 L). The filtrate was concentrated, transferred to a reaction flask, and water (66.0 L) was added. The suspension was stirred for a minimum of 30 min, filtered, and the filter cake was washed with water (90.0 L) until the pH of the rinse was 7-9. The solid was dried under vacuum at 50° C.±5° C. for a minimum of 12 h to give 8.58 kg (89.3%) of 2-((3-pyridinyl)methylene)-1-azabicyclo[2.2.2]octan-3-one.

### (2S)-2-((3-pyridinyl)methyl)-1-azabicyclo[2.2.2]octan-3-one di-p-toluoyl-D-tartrate salt

2-((3-Pyridinyl)methylene)-1-azabicyclo[2.2.2]octan-3-one (5.40 kg, 25.2 mol) and methanol (40.5 L) were added to a 72 L reaction vessel under an inert atmosphere equipped with a mechanical stirrer, temperature probe, low-pressure gas regulator system, and pressure gauge. The headspace was filled with nitrogen, and the mixture was stirred to obtain a clear yellow solution. To the flask was added 10% palladium on carbon (50% wet) (270 g). The atmosphere of the reactor was evacuated using a vacuum pump, and the headspace was replaced with hydrogen to 10 to 20 inches water pressure. The evacuation and pressurization with hydrogen were repeated 2 more times, leaving the reactor under 20 inches water pressure of hydrogen gas after the third pressurization. The reaction mixture was stirred at 20° C.±5° C. for a minimum of 12 h, and the reaction was monitored via TLC. Upon completion of the reaction, the suspension was filtered through a bed of Celite®545 (1.9 kg) on a sintered glass funnel, and the filter cake was washed with methanol (10.1 L). The filtrate was concentrated to obtain a semi-solid which was transferred, under an nitrogen atmosphere, to a 200 L reaction flask fitted with a mechanical stirrer, condenser, and temperature probe. The semi-solid was dissolved in ethanol (57.2 L), and di-p-toluoyl-D-tartaric acid (DTTA) (9.74 kg, 25.2 mol) was added. The stirring reaction mixture was heated at reflux for a minimum of 1 h, and for an additional minimum of 12 h while the reaction was cooled to between 15° C. and 30° C. The suspension was filtered using a tabletop filter, and the filter cake was washed with ethanol (11.4 L). The product was dried under vacuum at ambient temperature to obtain 11.6 kg (76.2% yield, 59.5% factored for purity) of (2S)-2-((3-pyridinyl)methyl)-1-azabicyclo[2.2.2]octan-3-one di-p-toluoyl-D-tartrate salt.

### (2S,3R)-3-Amino-2-((3-pyridinyl)methyl)-1-azabicyclo[2.2.2]octane di-p-toluoyl-D-tartrate salt

Water (46.25 L) and sodium bicarbonate (4.35 kg, 51.8 mol) were added to a 200 L flask. Upon complete dissolution, dichloromethane (69.4 L) was added. (2S)-2-((3-Pyridinyl)methyl)-1-azabicyclo[2.2.2]octan-3-one di-p-toluoyl-D-tartrate salt (11.56 kg, 19.19 mol) was added, and the reaction mixture was stirred for between 2 min and 10 min. The layers were allowed to separate for a minimum of 2 min (additional water (20 L) was added when necessary to partition the layers). The organic phase was removed and dried over anhydrous sodium sulfate. Dichloromethane (34.7 L) was added to the remaining aqueous phase, and the suspension was stirred for between 2 min and 10 min. The layers were allowed to separate for between 2 min and 10 min. Again, the organic phase was removed and dried over anhydrous sodium sulfate. The extraction of the aqueous phase with dichloromethane (34.7 L) was repeated one more time, as above. Samples of each extraction were submitted for chiral HPLC analysis. The sodium sulfate was removed by filtration, and the filtrates were concentrated to obtain (2S)-2-((3-pyridinyl)methyl)-1-azabicyclo[2.2.2]octan-3-one (4.0 kg) as a solid.

The (2S)-2-((3-pyridinyl)methyl)-1-azabicyclo[2.2.2]octan-3-one (3.8 kg) was transferred to a clean 100 L glass reaction flask, under a nitrogen atmosphere, fitted with a mechanical stirrer and temperature probe. Anhydrous tetrahydrofuran (7.24 L) and (+)-(R)-α-methylbenzylamine (2.55 L, 20.1 mol) were added. Titanium(IV) isopropoxide (6.47 L, 21.8 mol) was added to the stirred reaction mixture over a 1 h period. The reaction was stirred under a nitrogen atmosphere for a minimum of 12 h. Ethanol (36.17 L) was added to the reaction mixture. The reaction mixture was cooled to below −5° C., and sodium borohydride (1.53 kg, 40.5 mol) was added in portions, keeping the reaction temperature below 15° C. (this addition took several hours). The reaction mixture was then stirred at 15° C.±10° C. for a minimum of 1 h. The reaction was monitored by HPLC, and upon completion of the reaction (as indicated by less than 0.5% of (2S)-2-((3-pyridinyl)methyl)-1-azabicyclo[2.2.2]octan-3-one remaining), 2 M sodium hydroxide (15.99 L) was added and the mixture was stirred for a minimum of 10 min. The reaction mixture was filtered through a bed of Celite®545 in a tabletop funnel. The filter cake was washed with ethanol (15.23 L), and the filtrate was concentrated to obtain an oil.

The concentrate was transferred to a clean 100 L glass reaction flask equipped with a mechanical stirrer and temperature probe under an inert atmosphere. Water (1 L) was added, and the mixture was cooled to 0° C.±5° C. 2 M Hydrochloric acid (24 L) was added to the mixture to adjust the pH of the mixture to pH 1. The mixture was then stirred for a minimum of 10 min, and 2 M sodium hydroxide (24 L) was slowly added to adjust the pH of the mixture to pH 14. The mixture was stirred for a minimum of 10 min, and the aqueous phase was extracted with dichloromethane (3×15.23 L). The organic phases were dried over anhydrous sodium sulfate (2.0 kg), filtered, and concentrated to give (2S,3R)-N-((1R)-phenylethyl)-3-amino-2-((3-pyridinyl)methyl))-1-azabicyclo[2.2.2]octane (4.80 kg, 84.7% yield).

The (2S,3R)-N-((1R)-phenylethyl)-3-amino-2-((3-pyridinyl)methyl)-1-azabicyclo[2.2.2]octane was transferred to a 22 L glass flask equipped with a mechanical stirrer and temperature probe under an inert atmosphere. Water (4.8 L) was added, and the stirring mixture was cooled to 5° C.±5° C. Concentrated hydrochloric acid (2.97 L) was slowly added to the reaction flask, keeping the temperature of the mixture below 25° C. The resulting solution was transferred to a 72 L reaction flask containing ethanol (18 L), equipped with a mechanical stirrer, temperature probe, and condenser under an inert atmosphere. To the flask was added 10% palladium on carbon (50% wet) (311.1 g) and cyclohexene (14.36 L). The reaction mixture was heated at near-reflux for a minimum of 12 h, and the reaction was monitored by TLC. Upon completion of the reaction, the reaction mixture was cooled to below 45° C., and it was filtered through a bed of Celite®545 (1.2 kg) on a sintered glass funnel. The filter cake was rinsed with ethanol (3 L) and the filtrate was concentrated to obtain an aqueous phase. Water (500 mL) was added to the concentrated filtrate, and this combined aqueous layer was washed with methyl tert-butyl ether (MTBE) (2×4.79 L). 2 M Sodium hydroxide (19.5 L) was added to the aqueous phase to adjust the pH of the mixture to pH 14. The mixture was then stirred for a minimum of 10 min. The aqueous phase was extracted with chloroform (4×11.96 L), and the combined organic phases were dried over anhydrous sodium sulfate (2.34 kg). The filtrate was filtered and concentrated to obtain (2S,3R)-3-amino-2-((3-pyridinyl)methyl)-1-azabicyclo[2.2.2]octane (3.49 kg, >quantitative yield) as an oil.

The (2S,3R)-3-amino-2-((3-pyridinyl)methyl)-1-azabicyclo[2.2.2]octane was transferred to a clean 100 L reaction flask equipped with a mechanical stirrer, condenser, and temperature probe under an inert atmosphere. Ethanol (38.4 L) and di-p-toluoyl-D-tartaric acid (3.58 kg, 9.27 mol) were added. The reaction mixture was heated at gentle reflux for a minimum of 1 h. The reaction mixture was then stirred for a minimum of 12 h while it was cooled to between 15° C. and 30° C. The resulting suspension was filtered, and the filter cake was washed with ethanol (5.76 L). The filter cake was transferred to a clean 100 L glass reaction flask equipped with a mechanical stirrer, temperature probe, and condenser under an inert atmosphere. A 9:1 ethanol/water solution (30.7 L) was added, and the resulting slurry was heated at gentle reflux for a minimum of 1 h. The reaction mixture was then stirred for a minimum of 12 h while cooling to between 15° C. and 30° C. The mixture was filtered and the filter cake was washed with ethanol (5.76 L). The product was collected and dried under vacuum at 50° C.±5° C. for a minimum of 12 h to give 5.63 kg (58.1% yield) of (2S,3R)-3-amino-2-((3-pyridinyl)methyl)-1-azabicyclo[2.2.2]octane di-p-toluoyl-D-tartrate salt.

### Compound A: (2S,3R)-N-(2-((3-Pyridinyl)methyl)-1-azabicyclo[2.2.2]octan-3-yl)-5-methylthiophene-2-carboxamide

(2S,3R)-3-Amino-2-((3-pyridinyl)methyl)-1-azabicyclo[2.2.2]octane dip-toluoyl-D-tartrate salt (51.0 g, 84.5 mmol), water (125 mL), 2 M sodium hydroxide (150 mL) and chloroform (400 mL) were shaken together in a separatory funnel, and the chloroform layer was drawn off. The aqueous layer was extracted three more times with chloroform (2×200 mL, then 100 mL). The combined chloroform layers were washed with saturated aqueous sodium chloride, dried over anhydrous sodium sulfate and concentrated by rotary evaporation. High vacuum treatment left (2S,3R)-3-amino-2-((3-pyridinyl)methyl)-1-azabicyclo[2.2.2]octane (18.0 g) as an oil.

The (2S,3R)-3-amino-2-((3-pyridinyl)methyl)-1-azabicyclo[2.2.2]octane was transferred to a 1 L glass reaction flask under an inert atmosphere. Dichloromethane (500 mL), triethylamine (40 mL, 0.30 mol), 5-methylthiophene-2-carboxylic acid (13.5 g, 94.9 mmol) and O-(benzotriazol-1-yl)-N,N,N,1-tetramethyluronium hexafluorophosphate (HBTU) (36.0 g, 94.9 mmol) were added to the reaction mixture. The mixture was stirred overnight at ambient temperature, and at which time the reaction was complete by HPLC. Water (200 mL), 2 M sodium hydroxide (200 mL) were added to the reaction, and the resulting mixture was shaken. The dichloromethane layer and a 200 mL dichloromethane extract of the aqueous layer were combined and washed with saturated aqueous sodium chloride (200 mL), dried over anhydrous sodium sulfate and concentrated, by rotary evaporation, to give an oil (quantitative yield). Column chromatographic purification on silica gel, eluting with a methanol in ethyl acetate gradient, gave (2S,3R)-N-(2-((3-pyridinyl)methyl)-1-azabicyclo[2.2.2]octan-3-yl)-5-methylthiophene-2-carboxamide (22.6 g, 78.5% yield) as a powder.

### (2S,3R)-N-(2-((3-Pyridinyl)methyl)-1-azabicyclo[2.2.2]octan-3-yl)-5-(2-pyridinyl)thiophene-2-carboxamide

A sample of (2S,3R)-3-amino-2-((3-pyridinyl)methyl)-1-azabicyclo[2.2.2]octane (5.5 g, 25 mmol), generated as described above from (2S,3R)-3-Amino-2-((3-pyridinyl)methyl)-1-azabicyclo[2.2.2]octane di-p-toluoyl-D-tartrate salt, was transferred to a 500 mL glass reaction flask under an inert atmosphere. Dichloromethane (200 mL), triethylamine (10 mL, 72 mmol), 5-(2-pyridinyl)thiophene-2-carboxylic acid (6.0 g, 29 mmol) and O-(benzotriazol-1-yl)-N,N,N,1-tetramethyluronium hexafluorophosphate (HBTU) (11.1 g, 29.2 mmol) were added to the reaction mixture. The mixture was stirred overnight at ambient temperature, and at which time the reaction was complete by HPLC. Water (100 mL), 2 M sodium hydroxide (100 mL) were added to the reaction, and the resulting mixture was shaken. The dichloromethane layer and two 250 mL dichloromethane extracts of the aqueous layer were combined and washed with saturated aqueous sodium chloride (200 mL), dried over anhydrous sodium sulfate and concentrated, by rotary evaporation, to give an oil (quantitative yield). Column chromatographic purification on silica gel, eluting with a methanol in ethyl acetate gradient, gave (2S,3R)-N-(2-((3-pyridinyl)methyl)-1-azabicyclo[2.2.2]octan-3-yl)-5-(2-pyridinyl)thiophene-2-carboxamide (8.0 g, 80% yield) as a powder.

### (2S,3R)-N-(2-((3-Pyridinyl)methyl)-1-azabicyclo[2.2.2]octan-3-yl)benzofuran-2-carboxamide

Racemic N-(2-((3-pyridinyl)methyl-1-azabicyclo[2.2.2]oct-3-yl)benzofuran-2-carboxamide, a synthesis, and utility in medical treatment, is described in U.S. Pat. No. 6,953,855 to Mazurov et al, herein incorporated by reference.

Particular synthetic steps vary in their amenability to scale-up. Reactions are found lacking in their ability to be scaled-up for a variety of reasons, including safety concerns, reagent expense, difficult work-up or purification, reaction energetics (thermodynamics or kinetics), and reaction yield. Both small scale and large scale synthetic methods are herein described.

The scalable synthesis utilizes both the dynamic resolution of a racemizable ketone (2-((3-pyridinyl)methyl)-1-azabicyclo[2.2.2]octan-3-one) and the stereoselective reduction of the (R)-α-methylbenzylamine imine derivative (reductive amination) of the resolved ketone.

### Small Scale

### 2-((3-Pyridinyl)methylene)-1-azabicyclo[2.2.2]octan-3-one

Potassium hydroxide (56 g, 0.54 mole) was dissolved in methanol (420 mL). 3-Quinuclidinone hydrochloride (75 g, 0.49 mole) was added and the mixture was stirred for 30 min at ambient temperature. 3-Pyridinecarboxaldehyde (58 g, 0.54 mole) was added and the mixture stirred for 16 h at ambient temperature. The reaction mixture became yellow during this period, with solids caking on the walls of the flask. The solids were scraped from the walls and the chunks broken up. With rapid stirring, water (390 mL) was added. When the solids dissolved, the mixture was cooled at 4° C. overnight. The crystals were collected by filtration, washed with water, and air dried to obtain 80 g of yellow solid. A second crop (8 g) was obtained by concentration of the filtrate to −10% of its former volume and cooling at 4° C. overnight. Both crops were sufficiently pure for further transformation (88 g, 82% yield).

### 2-((3-Pyridinyl)methyl)-1-azabicyclo[2.2.2]octan-3-one

2-((3-Pyridinyl)methylene)-1-azabicyclo[2.2.2]octan-3-one (20 g, 93 mmol) was suspended in methanol (200 mL) and treated with 46 mL of 6 M hydrochloric acid. 10% Palladium on carbon (1.6 g) was added and the mixture was shaken under 25 psi hydrogen for 16 h. The mixture was filtered through diatomaceous earth, and the solvent was removed from the filtrate by rotary evaporation. This provided crude 2-((3-pyridinyl)methyl)-1-azabicyclo[2.2.2]octan-3-one hydrochloride, as a white gum (20 g), which was subsequently treated with 2 M sodium hydroxide (50 mL) and chloroform (50 mL) and stirred for an hour. The chloroform layer was separated, and the aqueous phase was treated with 2 M sodium hydroxide (−5 mL, enough to raise the pH to 10) and saturated aqueous Sodium chloride (25 mL). This aqueous mixture was extracted with chloroform (3×10 mL), and the combined chloroform extracts were dried (anhydrous magnesium sulfate) and concentrated by rotary evaporation. The residue (18 g) was dissolved in warm ether (320 mL) and cooled to 4° C. The white solid was filtered off, washed with a small portion of cold ether and air dried. Concentration of the filtrate to −10% of its former volume and cooling at 4° C. produced a second crop. A combined yield 16 g (79%) was obtained.

### 3-Amino-2-((3-pyridinyl)methyl)-1-azabicyclo[2.2.2]octane

To a stirred solution of 2-((3-pyridinyl)methyl)-1-azabicyclo[2.2.2]octan-3-one (3.00 g, 13.9 mmol) in dry methanol (20 mL), under nitrogen, was added a 1M solution of zinc chloride in ether (2.78 mL, 2.78 mmol). After stirring at ambient temperature for 30 min, this mixture was treated with solid ammonium formate (10.4 g, 167 mmol). After stirring another hour at ambient temperature, solid sodium cyanoborohydride (1.75 g, 27.8 mmol) was added in portions. The reaction was then stirred at ambient temperature overnight and terminated by addition of water (−5 mL). The quenched reaction was partitioned between 5 M sodium hydroxide (10 mL) and chloroform (20 mL). The aqueous layer was extracted with chloroform (20 mL), and combined organic layers were dried (sodium sulfate), filtered and concentrated. This left 2.97 g of yellow gum. GCMS analysis indicated that the product was a 1:9 mixture of the cis and trans amines, along with a trace of the corresponding alcohol (98% total mass recovery).

### (2R,3S) and (2S,3R)-3-amino-2-((3-Pyridinyl)methyl)-1-azabicyclo[2.2.2]octane

Di-p-toluoyl-D-tartaric acid (5.33 g, 13.8 mmol) was added to a stirred solution of crude 3-amino-2-((3-pyridinyl)methyl)-1-azabicyclo[2.2.2]octane (6.00 g, 27.6 mmol of 1:9 cis/trans) in methanol (20 mL). After complete dissolution, the clear solution was then concentrated to a solid mass by rotary evaporation. The solid was dissolved in a minimum amount of boiling methanol (−5 mL). The solution was cooled slowly, first to ambient temperature (1 h), then for ˜4 h at 5° C. and finally at −5° C. overnight. The precipitated salt was collected by suction filtration and recrystallized from 5 mL of methanol. Air drying left 1.4 g of white solid, which was partitioned between chloroform (5 mL) and 2 M sodium hydroxide (5 mL). The chloroform layer and a 5 mL chloroform extract of the aqueous layer were combined, dried (anhydrous sodium sulfate) and concentrated to give a colorless oil (0.434 g). The enantiomeric purity of this free base was determined by conversion of a portion into its N-(tert-butoxycarbonyl)-L-prolinamide, which was then analyzed for diastereomeric purity (98%) using LCMS.

The mother liquor from the initial crystallization was made basic (˜pH 11) with 2 M sodium hydroxide and extracted twice with chloroform (10 mL). The chloroform extracts were dried (anhydrous sodium sulfate) and concentrated to give an oil. This amine (3.00 g, 13.8 mmol) was dissolved in methanol (10 mL) and treated with di-p-toluoyl-L-tartaric acid (2.76 g, 6.90 mmol). The mixture was warmed to aid dissolution and then cooled slowly to −5° C., where it remained overnight. The precipitate was collected by suction filtration, recrystallized from methanol and dried. This left 1.05 g of white solid. The salt was converted into the free base (yield=0.364 g), and the enantiomeric purity (97%) was assessed using the prolinamide method, as described above for the other enantiomer.

### Trans isomer 1 of N-(2-((3-pyridinyl)methyl)-1-azabicyclo[2.2.2]octan-3-yl)benzofuran-2-carboxamide

Diphenylchlorophosphate (0.35 mL, 0.46 g, 1.7 mmol) was added drop-wise to a solution of benzofuran-2-carboxylic acid (0.28 g, 1.7 mmol) and triethylamine (0.24 mL, 0.17 g, 1.7 mmol) in dry dichloromethane (5 mL). After stirring at ambient temperature for 30 min, a solution of (2S,3R)-3-amino-2-((3-pyridinyl)methyl)-1-azabicyclo[2.2.2]octane (0.337 g, 1.55 mmol) (that derived from the di-p-toluoyl-D-tartaric acid salt) and triethylamine (0.24 mL, 0.17 g, 1.7 mmol) in dry dichloromethane (5 mL) was added. The reaction mixture was stirred overnight at ambient temperature, and then treated with 10% sodium hydroxide (1 mL). The biphasic mixture was separated, and the organic layer was concentrated on a Genevac centrifugal evaporator. The residue was dissolved in methanol (6 mL) and purified by HPLC on a C18 silica gel column, using an acetonitrile/water gradient, containing 0.05% trifluoroacetic acid, as eluent. Concentration of selected fractions, partitioning of the resulting residue between chloroform and saturated aqueous sodium bicarbonate, and evaporation of the chloroform gave 0.310 g (42% yield) of white powder (95% pure by GCMS). ¹H NMR (300 MHz, CDCl₃) δ 8.51 (d, 1H), 8.34 (dd, 1H), 7.66 (d, 1H), 7.58 (dt, 1H), 7.49 (d, 1H), 7.44 (s, 1H), 7.40 (dd, 1H), 7.29 (t, 1H), 7.13 (dd, 1H), 6.63 (d, 1H), 3.95 (t, 1H), 3.08 (m, 1H), 2.95 (m, 4H), 2.78 (m, 2H), 2.03 (m, 1H), 1.72 (m, 3H), 1.52 (m, 1H).

This material (trans enantiomer 1) was later determined to be identical, by chiral chromatographic analysis, to material whose absolute configuration is 2S,3R (established by x-ray crystallographic analysis).

### Trans isomer 2 of N-(2-((3-pyridinyl)methyl)-1-azabicyclo[2.2.2]octan-3-yl)benzofuran-2-carboxamide

Diphenylchlorophosphate (96 μL, 124 mg, 0.46 mmol) was added drop-wise to a solution of the benzofuran-2-carboxylic acid (75 mg, 0.46 mmol) (that derived from the di-p-toluoyl-L-tartaric acid salt) and triethylamine (64 μL, 46 mg, 0.46 mmol) in dry dichloromethane (1 mL). After stirring at ambient temperature for 45 min, a solution of (2R,3S)-3-amino-2-((3-pyridinyl)methyl)-1-azabicyclo[2.2.2]octane (0.10 g, 0.46 mmol) and triethylamine (64 μL, 46 mg, 0.46 mmol) in dry dichloromethane (1 mL) was added. The reaction mixture was stirred overnight at ambient temperature, and then treated with 10% sodium hydroxide (1 mL). The biphasic mixture was separated, and the organic layer and a chloroform extract (2 mL) of the aqueous layer was concentrated by rotary evaporation. The residue was dissolved in methanol and purified by HPLC on a C18 silica gel column, using an acetonitrile/water gradient, containing 0.05% trifluoroacetic acid, as eluent.

Concentration of selected fractions, partitioning of the resulting residue between chloroform and saturated aqueous sodium bicarbonate, and evaporation of the chloroform gave 82.5 mg (50% yield) of a white powder. The NMR spectrum was identical to that obtained for the (2S,3R) isomer.

Since the immediate precursor of this material (trans enantiomer 2) is enantiomeric to the immediate precursor of 2S,3R compound (trans enantiomer 1), the absolute configuration of trans enantiomer 2 is presumed to be 2R,3S.

### Large Scale

### 2-((3-Pyridinyl)methylene)-1-azabicyclo[2.2.2]octan-3-one

3-Quinuclidinone hydrochloride (8.25 kg, 51.0 mol) and methanol (49.5 L) were added to a 100 L glass reaction flask, under an nitrogen atmosphere, equipped with a mechanical stirrer, temperature probe, and condenser. Potassium hydroxide (5.55 kg, 99.0 mol) was added via a powder funnel over an approximately 30 min period, resulting in a rise in reaction temperature from 50° C. to 56° C. Over an approximately 2 h period, 3-pyridinecarboxaldehyde (4.80 kg, 44.9 mol) was added to the reaction mixture. The resulting mixture was stirred at 20° C.±5° C. for a minimum of 12 h, as the reaction was monitored by thin layer chromatography (TLC). Upon completion of the reaction, the reaction mixture was filtered through a sintered glass funnel and the filter cake was washed with methanol (74.2 L). The filtrate was concentrated, transferred to a reaction flask, and water (66.0 L) was added. The suspension was stirred for a minimum of 30 min, filtered, and the filter cake was washed with water (90.0 L) until the pH of the rinse was 7-9. The solid was dried under vacuum at 50° C.±5° C. for a minimum of 12 h to give 8.58 kg (89.3%) of 2-((3-pyridinyl)methylene)-1-azabicyclo[2.2.2]octan-3-one.

### (2S)-2-((3-pyridinyl)methyl)-1-azabicyclo[2.2.2]octan-3-one di-p-toluoyl-D-tartrate salt

2-((3-Pyridinyl)methylene)-1-azabicyclo[2.2.2]octan-3-one (5.40 kg, 25.2 mol) and methanol (40.5 L) were added to a 72 L reaction vessel under an inert atmosphere equipped with a mechanical stirrer, temperature probe, low-pressure gas regulator system, and pressure gauge. The headspace was filled with nitrogen, and the mixture was stirred to obtain a clear yellow solution. To the flask was added 10% palladium on carbon (50% wet) (270 g). The atmosphere of the reactor was evacuated using a vacuum pump, and the headspace was replaced with hydrogen to 10 to 20 inches water pressure. The evacuation and pressurization with hydrogen were repeated 2 more times, leaving the reactor under 20 inches water pressure of hydrogen gas after the third pressurization. The reaction mixture was stirred at 20° C.±5° C. for a minimum of 12 h, and the reaction was monitored via TLC. Upon completion of the reaction, the suspension was filtered through a bed of Celite®545 (1.9 kg) on a sintered glass funnel, and the filter cake was washed with methanol (10.1 L). The filtrate was concentrated to obtain a semi-solid which was transferred, under an nitrogen atmosphere, to a 200 L reaction flask fitted with a mechanical stirrer, condenser, and temperature probe. The semi-solid was dissolved in ethanol (57.2 L), and di-p-toluoyl-D-tartaric acid (DTTA) (9.74 kg, 25.2 mol) was added. The stirring reaction mixture was heated at reflux for a minimum of 1 h, and for an additional minimum of 12 h while the reaction was cooled to between 15° C. and 30° C. The suspension was filtered using a tabletop filter, and the filter cake was washed with ethanol (11.4 L). The product was dried under vacuum at ambient temperature to obtain 11.6 kg (76.2% yield, 59.5% factored for purity) of (2S)-2-((3-pyridinyl)methyl)-1-azabicyclo[2.2.2]octan-3-one di-p-toluoyl-D-tartrate salt.

### (2S,3R)-3-Amino-2-((3-pyridinyl)methyl)-1-azabicyclo[2.2.2]octane di-p-toluoyl-D-tartrate salt

Water (46.25 L) and sodium bicarbonate (4.35 kg, 51.8 mol) were added to a 200 L flask. Upon complete dissolution, dichloromethane (69.4 L) was added. (2S)-2-((3-Pyridinyl)methyl)-1-azabicyclo[2.2.2]octan-3-one di-p-toluoyl-D-tartrate salt (11.56 kg, 19.19 mol) was added, and the reaction mixture was stirred for between 2 min and 10 min. The layers were allowed to separate for a minimum of 2 min (additional water (20 L) was added when necessary to partition the layers). The organic phase was removed and dried over anhydrous sodium sulfate. Dichloromethane (34.7 L) was added to the remaining aqueous phase, and the suspension was stirred for between 2 min and 10 min. The layers were allowed to separate for between 2 min and 10 min. Again, the organic phase was removed and dried over anhydrous sodium sulfate. The extraction of the aqueous phase with dichloromethane (34.7 L) was repeated one more time, as above. Samples of each extraction were submitted for chiral HPLC analysis. The sodium sulfate was removed by filtration, and the filtrates were concentrated to obtain (2S)-2-((3-pyridinyl)methyl)-1-azabicyclo[2.2.2]octan-3-one (4.0 kg) as a solid. The (2S)-2-((3-pyridinyl)methyl)-1-azabicyclo[2.2.2]octan-3-one (3.8 kg) was transferred to a clean 100 L glass reaction flask, under a nitrogen atmosphere, fitted with a mechanical stirrer and temperature probe. Anhydrous tetrahydrofuran (7.24 L) and (+)-(R)-α-methylbenzylamine (2.55 L, 20.1 mol) were added. Titanium(IV) isopropoxide (6.47 L, 21.8 mol) was added to the stirred reaction mixture over a 1 h period. The reaction was stirred under a nitrogen atmosphere for a minimum of 12 h. Ethanol (36.17 L) was added to the reaction mixture. The reaction mixture was cooled to below −5° C., and sodium borohydride (1.53 kg, 40.5 mol) was added in portions, keeping the reaction temperature below 15° C. (this addition took several hours). The reaction mixture was then stirred at 15° C.±10° C. for a minimum of 1 h. The reaction was monitored by HPLC, and upon completion of the reaction (as indicated by less than 0.5% of (2S)-2-((3-pyridinyl)methyl)-1-azabicyclo[2.2.2]octan-3-one remaining), 2 M sodium hydroxide (15.99 L) was added and the mixture was stirred for a minimum of 10 min. The reaction mixture was filtered through a bed of Celite®545 in a tabletop funnel. The filter cake was washed with ethanol (15.23 L), and the filtrate was concentrated to obtain an oil.

The concentrate was transferred to a clean 100 L glass reaction flask equipped with a mechanical stirrer and temperature probe under an inert atmosphere. Water (1 L) was added, and the mixture was cooled to 0° C.±5° C. 2 M Hydrochloric acid (24 L) was added to the mixture to adjust the pH of the mixture to pH 1. The mixture was then stirred for a minimum of 10 min, and 2 M sodium hydroxide (24 L) was slowly added to adjust the pH of the mixture to pH 14. The mixture was stirred for a minimum of 10 min, and the aqueous phase was extracted with dichloromethane (3×15.23 L). The organic phases were dried over anhydrous sodium sulfate (2.0 kg), filtered, and concentrated to give (2S,3R)-N-((1R)-phenylethyl)-3-amino-2-((3-pyridinyl)methyl))-1-azabicyclo[2.2.2]octane (4.80 kg, 84.7% yield).

The (2S,3R)-N-((1R)-phenylethyl)-3-amino-2-((3-pyridinyl)methyl)-1-azabicyclo[2.2.2]octane was transferred to a 22 L glass flask equipped with a mechanical stirrer and temperature probe under an inert atmosphere. Water (4.8 L) was added, and the stirring mixture was cooled to 5° C.±5° C. Concentrated hydrochloric acid (2.97 L) was slowly added to the reaction flask, keeping the temperature of the mixture below 25° C. The resulting solution was transferred to a 72 L reaction flask containing ethanol (18 L), equipped with a mechanical stirrer, temperature probe, and condenser under an inert atmosphere. To the flask was added 10% palladium on carbon (50% wet) (311.1 g) and cyclohexene (14.36 L). The reaction mixture was heated at near-reflux for a minimum of 12 h, and the reaction was monitored by TLC. Upon completion of the reaction, the reaction mixture was cooled to below 45° C., and it was filtered through a bed of Celite®545 (1.2 kg) on a sintered glass funnel. The filter cake was rinsed with ethanol (3 L) and the filtrate was concentrated to obtain an aqueous phase. Water (500 mL) was added to the concentrated filtrate, and this combined aqueous layer was washed with methyl tert-butyl ether (MTBE) (2×4.79 L). 2 M Sodium hydroxide (19.5 L) was added to the aqueous phase to adjust the pH of the mixture to pH 14. The mixture was then stirred for a minimum of 10 min. The aqueous phase was extracted with chloroform (4×11.96 L), and the combined organic phases were dried over anhydrous sodium sulfate (2.34 kg). The filtrate was filtered and concentrated to obtain (2S,3R)-3-amino-2-((3-pyridinyl)methyl)-1-azabicyclo[2.2.2]octane (3.49 kg, >quantitative yield) as an oil.

The (2S,3R)-3-amino-2-((3-pyridinyl)methyl)-1-azabicyclo[2.2.2]octane was transferred to a clean 100 L reaction flask equipped with a mechanical stirrer, condenser, and temperature probe under an inert atmosphere. Ethanol (38.4 L) and di-p-toluoyl-D-tartaric acid (3.58 kg, 9.27 mol) were added. The reaction mixture was heated at gentle reflux for a minimum of 1 h. The reaction mixture was then stirred for a minimum of 12 h while it was cooled to between 15° C. and 30° C. The resulting suspension was filtered, and the filter cake was washed with ethanol (5.76 L). The filter cake was transferred to a clean 100 L glass reaction flask equipped with a mechanical stirrer, temperature probe, and condenser under an inert atmosphere. A 9:1 ethanol/water solution (30.7 L) was added, and the resulting slurry was heated at gentle reflux for a minimum of 1 h. The reaction mixture was then stirred for a minimum of 12 h while cooling to between 15° C. and 30° C. The mixture was filtered and the filter cake was washed with ethanol (5.76 L). The product was collected and dried under vacuum at 50° C.±5° C. for a minimum of 12 h to give 5.63 kg (58.1% yield) of (2S,3R)-3-amino-2-((3-pyridinyl)methyl)-1-azabicyclo[2.2.2]octane di-p-toluoyl-D-tartrate salt.

### (2S,3R)-N-(2-((3-Pyridinyl)methyl)-1-azabicyclo[2.2.2]octan-3-yl)benzofuran-2-carboxamide

(2S,3R)-3-Amino-2-((3-pyridinyl)methyl)-1-azabicyclo[2.2.2]octane di-p-toluoyl-D-tartrate salt (3.64 kg, 5.96 mol) and 10% aqueous sodium chloride solution (14.4 L, 46.4 mol) were added to a 72 L glass reaction flask equipped with a mechanical stirrer under an inert atmosphere. 5 M Sodium hydroxide (5.09 L) was added to the stirring mixture to adjust the pH of the mixture to pH 14. The mixture was then stirred for a minimum of 10 min. The aqueous solution was extracted with chloroform (4×12.0 L), and the combined organic layers were dried over anhydrous sodium sulfate (1.72 kg). The combined organic layers were filtered, and the filtrate was concentrated to obtain (2S,3R)-3-amino-2-((3-pyridinyl)methyl)-1-azabicyclo[2.2.2]octane (1.27 kg) as an oil.

The (2S,3R)-3-amino-2-((3-pyridinyl)methyl)-1-azabicyclo[2.2.2]octane was transferred to a 50 L glass reaction flask equipped with a mechanical stirrer under an inert atmosphere. Dichloromethane (16.5 L), triethylamine (847 mL, 6.08 mol), benzofuran-2-carboxylic acid (948 g, 5.85 mol) and O-(benzotriazol-1-yl)-N,N,N,1-tetramethyluronium hexafluorophosphate (HBTU) (2.17 kg, 5.85 mol) were added to the reaction mixture. The mixture was stirred for a minimum of 4 h at ambient temperature, and the reaction was monitored by HPLC. Upon completion of the reaction, 10% aqueous potassium carbonate (12.7 L, 17.1 mol) was added to the reaction mixture and the mixture was stirred for a minimum of 5 min. The layers were separated and the organic phase was washed with 10% brine (12.7 L). The layers were separated and the organic phase was cooled to 15° C.±10° C. 3 M Hydrochloric acid (8.0 L) was slowly added to the reaction mixture to adjust the pH of the mixture to pH 1. The mixture was then stirred for a minimum of 5 min, and the layers were allowed to partition for a minimum of 5 min. The solids were filtered using a table top filter. The layers of the filtrate were separated, and the aqueous phase and the solids from the funnel were transferred to the reaction flask. 3 M Sodium hydroxide (9.0 L) was slowly added to the flask in portions to adjust the pH of the mixture to pH 14. The aqueous phase was extracted with dichloromethane (2×16.5 L). The combined organic phases were dried over anhydrous sodium sulfate (1.71 kg). The mixture was filtered, and the filtrate was concentrated to give (2S,3R)-N-(2-((3-pyridinyl)methyl)-1-azabicyclo[2.2.2]octan-3-yl)benzofuran-2-carboxamide (1.63 kg, 77.0% yield) as a yellow solid.

### (2S,3R)-N-(2-((3-pyridinyl)methyl)-1-azabicyclo[2.2.2]oct-3-yl)benzofuran-2-carboxamide p-toluenesulfonate

(2S,3R)-N-(2-((3-Pyridinyl)methyl)-1-azabicyclo[2.2.2]octan-3-yl)benzofuran-2-carboxamide (1.62 kg, 4.48 mol) and dichloromethane (8.60 kg) were added into a carboy. The weight/weight percent of the material in solution was determined through HPLC analysis. The solution was concentrated to an oil, acetone (4 L) was added, and the mixture was concentrated to an oily solid. Additional acetone (12 L) was added to the oily solid in the rotary evaporator bulb, and the resulting slurry was transferred to a 50 L glass reaction flask with a mechanical stirrer, condenser, temperature probe, and condenser under an inert atmosphere. The reaction mixture was heated to 50° C.±5° C. Water (80.7 g) was added to the solution, and it was stirred for a minimum of 10 min. p-Toluenesulfonic acid (853 g, 4.44 mol) was added to the reaction mixture in portions over approximately 15 min. The reaction mixture was heated to reflux and held at that temperature for a minimum of 30 min to obtain a solution. The reaction was cooled to 40° C.±5° C. over approximately 2 h. Isopropyl acetate (14.1 L) was added over approximately 1.5 h. The reaction mixture was slowly cooled to ambient temperature over a minimum of 10 h. The mixture was filtered and the filter cake was washed with isopropyl acetate (3.5 L). The isolated product was dried under vacuum at 105° C.±5° C. for between 2 h and 9 h to give 2.19 kg (88.5% yield) of (2S,3R)-N-(2-((3-pyridinyl)methyl)-1-azabicyclo[2.2.2]oct-3-yl)benzofuran-2-carboxamide p-toluenesulfonate, mp 226-228° C. ¹H NMR (500 MHz, D₂O) δ 8.29 (s, 1H), 7.78 (m, J=5.1, 1H), 7.63 (d, J=7.9, 1H), 7.54 (d, J=7.8, 1H), 7.49 (d, J=8.1, 2H), 7.37 (m, J=8.3, 1H), 7.33 (m, J=8.3, 6.9, 1.0, 1H), 7.18 (m, J=7.8, 6.9, 1.0, 1H), 7.14 (d, J=8.1, 2H), 7.09 (s, 1H), 6.99 (dd, J=7.9, 5.1, 1H), 4.05 (m, J=7.7, 1H), 3.74 (m, 1H), 3.47 (m, 2H), 3.28 (m, 1H), 3.22 (m, 1H), 3.15 (dd, J=13.2, 4.7, 1H), 3.02 (dd, J=13.2, 11.5, 1H), 2.19 (s, 3H), 2.02 (m, 2H), 1.93 (m, 2H), 1.79 (m, 1H). ¹³C NMR (126 MHz, D₂O) δ 157.2, 154.1, 150.1, 148.2, 146.4, 145.2, 138.0, 137.0, 130.9, 128.2 (2), 126.9, 126.8, 125.5 (2), 123.7, 123.3, 122.7, 111.7, 100.7, 61.3, 50.2, 48.0, 40.9, 33.1, 26.9, 21.5, 20.8, 17.0.

Samples of this material were converted into free base (for use in salt selection studies) by treatment with aqueous sodium hydroxide and extraction with chloroform. Thorough evaporation of the chloroform left an off-white powder, mp 167-170° C., with the following spectral characteristics: Positive ion electrospray MS [M+H]⁺ ion m/z=362. ¹H NMR (500 MHz, DMSO-d₆) δ 8.53 (d, J=7.6 Hz, 1H), 8.43 (d, J=1.7 Hz, 1H), 8.28 (dd, J=1.6, 4.7 Hz, 1H), 7.77 (d, J=7.7 Hz, 1H), 7.66 (d, J=8.5 Hz, 1H), 7.63 (dt, J=1.7, 7.7 Hz, 1H), 7.52 (s, 1H), 7.46 (m, J=8.5, 7.5 Hz, 1H), 7.33 (m, J=7.7, 7.5 Hz, 1H), 7.21 (dd, J=4.7, 7.7 Hz, 1H), 3.71 (m, J=7.6 Hz, 1H), 3.11 (m, 1H), 3.02 (m, 1H), 2.80 (m, 2H), 2.69 (m, 2H), 2.55 (m, 1H), 1.80 (m, 1H), 1.77 (m, 1H), 1.62 (m, 1H), 1.56 (m, 1H), 1.26 (m, 1H). ¹³C NMR (126 MHz, DMSO-d₆) δ 158.1, 154.1, 150.1, 149.1, 146.8, 136.4, 135.4, 127.1, 126.7, 123.6, 122.9, 122.6, 111.8, 109.3, 61.9, 53.4, 49.9, 40.3, 35.0, 28.1, 26.1, 19.6.

The monohydrochloride salt (see Example 5) was submitted for x-ray crystallographic analysis. The resulting crystal structure established the 2S,3R absolute configuration.

### Example 5

### Synthesis of (2S,3R)-N-(2-((3-pyridinyl)methyl)-1-azabicyclo[2.2.2]oct-3-yl)benzofuran-2-carboxamide hydrochloride salt

A hydrochloric acid/THF solution was prepared by adding of concentrated hydrochloric acid (1.93 mL of 12M, 23.2 mmol) drop-wise to 8.5 mL of chilled THF. The solution was warmed to ambient temperature. To a round bottom flask was added (2S,3R)-N-(2-((3-pyridinyl)methyl)-1-azabicyclo[2.2.2]oct-3-yl)benzofuran-2-carboxamide (8.49 g, 23.5 mmol) and acetone (85 mL). The mixture was stirred and heated at 45-50° C. until a complete solution was obtained. The hydrochloric acid/THF solution prepared above was added drop-wise over a 5 min period, with additional THF (1.5 mL) used in the transfer. Granular, white solids began to form during the addition of the acid solution. The mixture was cooled to ambient temperature, and stirred overnight (16 h). The solids were collected by suction filtration, the filter cake was washed with acetone (10 mL), and the solid was air-dried with suction for 30 min. The solid was further dried in a vacuum oven at 75° C. for 2 h to give 8.79 g of the fine white crystals (94% yield), mp 255-262° C. Chiral LC analysis gave a purity of 98.8% (270 nm). ¹H-NMR (DMSO-d₆) shows no residual solvents and confirms mono stoichiometry. ¹H NMR (300 MHz, DMSO-d₆) δ 10.7 (broad s, 1H—quaternary ammonium), 8.80 (broad s, 1H—amide H), 8.54 (s, 1H), 8.23 (d, 1H), 7.78 (d, 1H), 7.74 (d, 1H), 7.60 (d, 1H), 7.47 (m, 2H), 7.33 (m, 1H), 7.19 (m, 1H), 4.19 (m, 1H), 4.08 (m, 1H), 3.05-3.55 (m, 6H), 2.00-2.10 (m, 3H), 1.90 (m, 1H), 1.70 (m, 1H). An x-ray crystallographic analysis of this salt established stereochemical assignment and stoichiometry.

### Biological Examples

As used herein, an “agonist” is a substance that stimulates its binding partner, typically a receptor. Stimulation is defined in the context of the particular assay, or may be apparent in the literature from a discussion herein that makes a comparison to a factor or substance that is accepted as an “agonist” or an “antagonist” of the particular binding partner under substantially similar circumstances as appreciated by those of skill in the art. Stimulation may be defined with respect to an increase in a particular effect or function that is induced by interaction of the agonist or partial agonist with a binding partner and can include allosteric effects.

As used herein, an “antagonist” is a substance that inhibits its binding partner, typically a receptor. Inhibition is defined in the context of the particular assay, or may be apparent in the literature from a discussion herein that makes a comparison to a factor or substance that is accepted as an “agonist” or an “antagonist” of the particular binding partner under substantially similar circumstances as appreciated by those of skill in the art. Inhibition may be defined with respect to a decrease in a particular effect or function that is induced by interaction of the antagonist with a binding partner, and can include allosteric effects.

As used herein, a “partial agonist” or a “partial antagonist” is a substance that provides a level of stimulation or inhibition, respectively, to its binding partner that is not fully or completely agonistic or antagonistic, respectively. It will be recognized that stimulation, and hence, inhibition is defined intrinsically for any substance or category of substances to be defined as agonists, antagonists, or partial agonists.

As used herein, “intrinsic activity” or “efficacy” relates to some measure of biological effectiveness of the binding partner complex. With regard to receptor pharmacology, the context in which intrinsic activity or efficacy should be defined will depend on the context of the binding partner (e.g., receptor/ligand) complex and the consideration of an activity relevant to a particular biological outcome. For example, in some circumstances, intrinsic activity may vary depending on the particular second messenger system involved. See Hoyer, D. and Boddeke, H., Trends Pharmacol. Sci. 14(7): 270-5 (1993), herein incorporated by reference with regard to such teaching. Where such contextually specific evaluations are relevant, and how they might be relevant in the context of the present invention, will be apparent to one of ordinary skill in the art.

As used herein, modulation of a receptor includes agonism, partial agonism, antagonism, partial antagonism, or inverse agonism of a receptor.

As used herein, neurotransmitters whose release is mediated by the compounds described herein include, but are not limited to, acetylcholine, dopamine, norepinephrine, serotonin and glutamate, and the compounds described herein function as modulators at the alpha7 or alpha4beta2 or both subtype of the CNS NNRs.

### CNS Disorders

As is appreciated, based upon the nAChR pharmacology of the compounds herein described, the compounds and their pharmaceutical compositions are useful in the treatment or prevention of a variety of CNS disorders, including neurodegenerative disorders, neuropsychiatric disorders, neurologic disorders, and addictions. The compounds and their pharmaceutical compositions can be used to treat or prevent cognitive deficits and dysfunctions, age-related and otherwise; attention disorders and dementias, including those due to infectious agents or metabolic disturbances; to provide neuroprotection; to treat convulsions and multiple cerebral infarcts; to treat mood disorders, compulsions and addictive behaviors; to provide analgesia; to control inflammation, such as mediated by cytokines and nuclear factor kappa B; to treat inflammatory disorders; to provide pain relief; and to treat infections, as anti-infectious agents for treating bacterial, fungal, and viral infections. Among the disorders, diseases and conditions that the compounds and pharmaceutical compositions of the present invention can be used to treat or prevent are: age-associated memory impairment (AAMI), mild cognitive impairment (MCI), age-related cognitive decline (ARCD), pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism including Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, tardive dyskinesia, hyperkinesia, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), and chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, oppositional defiant disorder, Tourette's syndrome, autism, drug and alcohol addiction, tobacco addiction, obesity, cachexia, psoriasis, lupus, acute cholangitis, aphthous stomatitis, ulcers, asthma, ulcerative colitis, inflammatory bowel disease, Crohn's disease, spastic dystonia, diarrhea, constipation, pouchitis, viral pneumonitis, arthritis, including, rheumatoid arthritis and osteoarthritis, endotoxaemia, sepsis, atherosclerosis, idiopathic pulmonary fibrosis, acute pain, chronic pain, neuropathies, urinary incontinence, diabetes, and neoplasias.

Cognitive impairments or dysfunctions may be associated with psychiatric disorders or conditions, such as schizophrenia and other psychotic disorders, including but not limited to psychotic disorder, schizophreniform disorder, schizoaffective disorder, delusional disorder, brief psychotic disorder, shared psychotic disorder, and psychotic disorders due to a general medical conditions, dementias and other cognitive disorders, including but not limited to mild cognitive impairment, pre-senile dementia, Alzheimer's disease, senile dementia, dementia of the Alzheimer's type, age-related memory impairment, Lewy body dementia, vascular dementia, AIDS dementia complex, dyslexia, Parkinsonism including Parkinson's disease, cognitive impairment and dementia of Parkinson's Disease, cognitive impairment of multiple sclerosis, cognitive impairment caused by traumatic brain injury, dementias due to other general medical conditions, anxiety disorders, including but not limited to panic disorder without agoraphobia, panic disorder with agoraphobia, agoraphobia without history of panic disorder, specific phobia, social phobia, obsessive-compulsive disorder, post-traumatic stress disorder, acute stress disorder, generalized anxiety disorder and generalized anxiety disorder due to a general medical condition, mood disorders, including but not limited to major depressive disorder, dysthymic disorder, bipolar depression, bipolar mania, bipolar I disorder, depression associated with manic, depressive or mixed episodes, bipolar II disorder, cyclothymic disorder, and mood disorders due to general medical conditions, sleep disorders, including but not limited to dyssomnia disorders, primary insomnia, primary hypersomnia, narcolepsy, parasomnia disorders, nightmare disorder, sleep terror disorder and sleepwalking disorder, mental retardation, learning disorders, motor skills disorders, communication disorders, pervasive developmental disorders, attention-deficit and disruptive behavior disorders, attention deficit disorder, attention deficit hyperactivity disorder, feeding and eating disorders of infancy, childhood, or adults, tic disorders, elimination disorders, substance-related disorders, including but not limited to substance dependence, substance abuse, substance intoxication, substance withdrawal, alcohol-related disorders, amphetamine or amphetamine-like-related disorders, caffeine-related disorders, cannabis-related disorders, cocaine-related disorders, hallucinogen-related disorders, inhalant-related disorders, nicotine-related disorders, opioid-related disorders, phencyclidine or phencyclidine-like-related disorders, and sedative-, hypnotic- or anxiolytic-related disorders, personality disorders, including but not limited to obsessive-compulsive personality disorder and impulse-control disorders.

The above conditions and disorders are discussed in further detail, for example, in the American Psychiatric Association: Diagnostic and Statistical Manual of Mental Disorders, Fourth Edition, Text Revision, Washington, D.C., American Psychiatric Association, 2000; incorporated herein by reference with regard to defining such conditions and disorders. This Manual may also be referred to for greater detail on the symptoms and diagnostic features associated with substance use, abuse, and dependence.

### Inflammation

The nervous system, primarily through the vagus nerve, is known to regulate the magnitude of the innate immune response by inhibiting the release of macrophage tumor necrosis factor (TNF). This physiological mechanism is known as the “cholinergic anti-inflammatory pathway” (see, for example, Tracey, “The inflammatory reflex,” Nature 420: 853-9 (2002)). Excessive inflammation and tumor necrosis factor synthesis cause morbidity and even mortality in a variety of diseases. These diseases include, but are not limited to, endotoxemia, rheumatoid arthritis, osteoarthritis, psoriasis, asthma, atherosclerosis, idiopathic pulmonary fibrosis, and inflammatory bowel disease.

Inflammatory conditions that can be treated or prevented by administering the compounds described herein include, but are not limited to, chronic and acute inflammation, psoriasis, endotoxemia, gout, acute pseudogout, acute gouty arthritis, arthritis, rheumatoid arthritis, osteoarthritis, allograft rejection, chronic transplant rejection, asthma, atherosclerosis, mononuclear-phagocyte dependent lung injury, idiopathic pulmonary fibrosis, atopic dermatitis, chronic obstructive pulmonary disease, adult respiratory distress syndrome, acute chest syndrome in sickle cell disease, inflammatory bowel disease, Crohn's disease, ulcerative colitis, acute cholangitis, aphteous stomatitis, pouchitis, glomerulonephritis, lupus nephritis, thrombosis, and graft vs. host reaction.

Inflammatory Response Associated with Bacterial and/or Viral Infection

Many bacterial and/or viral infections are associated with side effects brought on by the formation of toxins, and the body's natural response to the bacteria or virus and/or the toxins. As discussed above, the body's response to infection often involves generating a significant amount of TNF and/or other cytokines. The over-expression of these cytokines can result in significant injury, such as septic shock (when the bacteria is sepsis), endotoxic shock, urosepsis and toxic shock syndrome.

Cytokine expression is mediated by NNRs, and can be inhibited by administering agonists or partial agonists of these receptors. Those compounds described herein that are agonists or partial agonists of these receptors can therefore be used to minimize the inflammatory response associated with bacterial infection, as well as viral and fungal infections. Examples of such bacterial infections include anthrax, botulism, and sepsis. Some of these compounds may also have antimicrobial properties.

These compounds can also be used as adjunct therapy in combination with existing therapies to manage bacterial, viral and fungal infections, such as antibiotics, antivirals and antifungals. Antitoxins can also be used to bind to toxins produced by the infectious agents and allow the bound toxins to pass through the body without generating an inflammatory response. Examples of antitoxins are disclosed, for example, in U.S. Pat. No. 6,310,043 to Bundle et al., incorporated herein by reference. Other agents effective against bacterial and other toxins can be effective and their therapeutic effect can be complemented by co-administration with the compounds described herein.

### Pain

The compounds can be administered to treat and/or prevent pain, including acute, neurologic, inflammatory, neuropathic and chronic pain. The analgesic activity of compounds described herein can be demonstrated in models of persistent inflammatory pain and of neuropathic pain, performed as described in U.S. Published Patent Application No. 20010056084 A1 (Allgeier et al.) (e.g., mechanical hyperalgesia in the complete Freund's adjuvant rat model of inflammatory pain and mechanical hyperalgesia in the mouse partial sciatic nerve ligation model of neuropathic pain).

The analgesic effect is suitable for treating pain of various genesis or etiology, in particular in treating inflammatory pain and associated hyperalgesia and/or allodynia, neuropathic pain and associated hyperalgesia and/or allodynia, chronic pain (e.g., severe chronic pain, post-operative pain and pain associated with various conditions including cancer, angina, renal or biliary colic, menstruation, migraine and gout). Inflammatory pain may be of diverse genesis, including arthritis and rheumatoid disease, teno-synovitis and vasculitis. Neuropathic pain includes trigeminal or herpetic neuralgia, diabetic neuropathy pain, causalgia, low back pain and deafferentation syndromes such as brachial plexus avulsion.

### Neovascularization

The alpha7 NNR is associated with neovascularization. Inhibition of neovascularization, for example, by administering antagonists (or at certain dosages, partial agonists) of the alpha7 NNR can treat or prevent conditions characterized by undesirable neovascularization or angiogenesis. Such conditions can include those characterized by inflammatory angiogenesis and/or ischemia-induced angiogenesis. Neovascularization associated with tumor growth can also be inhibited by administering those compounds described herein that function as antagonists or partial agonists of alpha7 NNR.

Specific antagonism of alpha7 NNR-specific activity reduces the angiogenic response to inflammation, ischemia, and neoplasia. Guidance regarding appropriate animal model systems for evaluating the compounds described herein can be found, for example, in Heeschen, C. et al., “A novel angiogenic pathway mediated by non-neuronal nicotinic acetylcholine receptors,” J. Clin. Invest 110(4):527-36 (2002), incorporated herein by reference regarding disclosure of alpha7-specific inhibition of angiogenesis and cellular (in vitro) and animal modeling of angiogenic activity relevant to human disease, especially the Lewis lung tumor model (in vivo, in mice—see, in particular, pages 529, and 532-533).

Representative tumor types that can be treated using the compounds described herein include NSCLC, ovarian cancer, pancreatic cancer, breast carcinoma, colon carcinoma, rectum carcinoma, lung carcinoma, oropharynx carcinoma, hypopharynx carcinoma, esophagus carcinoma, stomach carcinoma, pancreas carcinoma, liver carcinoma, gallbladder carcinoma, bile duct carcinoma, small intestine carcinoma, urinary tract carcinoma, kidney carcinoma, bladder carcinoma, urothelium carcinoma, female genital tract carcinoma, cervix carcinoma, uterus carcinoma, ovarian carcinoma, choriocarcinoma, gestational trophoblastic disease, male genital tract carcinoma, prostate carcinoma, seminal vesicles carcinoma, testes carcinoma, germ cell tumors, endocrine gland carcinoma, thyroid carcinoma, adrenal carcinoma, pituitary gland carcinoma, skin carcinoma, hemangiomas, melanomas, sarcomas, bone and soft tissue sarcoma, Kaposi's sarcoma, tumors of the brain, tumors of the nerves, tumors of the eyes, tumors of the meninges, astrocytomas, gliomas, glioblastomas, glioblastoma multiforme, including giant cell glioblastoma and gliosarcoma, retinoblastomas, neuromas, neuroblastomas, Schwannomas, meningiomas, solid tumors arising from hematopoietic malignancies (such as leukemias, chloromas, plasmacytomas and the plaques and tumors of mycosis fungoides and cutaneous T-cell lymphoma/leukemia), and solid tumors arising from lymphomas.

The compounds can also be administered in conjunction with other forms of anti-cancer treatment, including co-administration with antineoplastic antitumor agents such as cis-platin, adriamycin, daunomycin, and the like, and/or anti-VEGF (vascular endothelial growth factor) agents, as such are known in the art.

The compounds can be administered in such a manner that they are targeted to the tumor site. For example, the compounds can be administered in microspheres, microparticles or liposomes conjugated to various antibodies that direct the microparticles to the tumor. Additionally, the compounds can be present in microspheres, microparticles or liposomes that are appropriately sized to pass through the arteries and veins, but lodge in capillary beds surrounding tumors and administer the compounds locally to the tumor. Such drug delivery devices are known in the art.

### Other Disorders

In addition to treating CNS disorders, inflammation, and undesirable neovascularization, and pain, the compounds of the present invention can be also used to prevent or treat certain other conditions, diseases, and disorders in which NNRs play a role. Examples include autoimmune disorders such as Lupus, disorders associated with cytokine release, cachexia secondary to infection (e.g., as occurs in AIDS, AIDS related complex and neoplasia), obesity, pemphitis, urinary incontinence, retinal diseases, infectious diseases, myasthenia, Eaton-Lambert syndrome, hypertension, osteoporosis, vasoconstriction, vasodilatation, cardiac arrhythmias, type I diabetes, bulimia, anorexia as well as those indications set forth in published PCT application WO 98/25619, herein incorporated by reference with regard to such disorders. The compounds of this invention can also be administered to treat convulsions such as those that are symptomatic of epilepsy, and to treat conditions such as syphillis and Creutzfeld-Jakob disease.

As presented, alpha7 compounds may be used in the treatment of a variety of disorders and conditions and, as such, may be used in combination with a variety of other suitable therapeutic agents useful in the treatment or prophylaxis of those disorders or conditions. Thus, one embodiment of the present invention includes the administration with other therapeutic compounds. For example, the compound of the present invention can be used in combination with other NNR ligands (such as varenicline), allosteric modulators of NNRs, antioxidants (such as free radical scavenging agents), antibacterial agents (such as penicillin antibiotics), antiviral agents (such as nucleoside analogs, like zidovudine and acyclovir), anticoagulants (such as warfarin), anti-inflammatory agents (such as NSAIDs), anti-pyretics, analgesics, anesthetics (such as used in surgery), acetylcholinesterase inhibitors (such as donepezil and galantamine), antipsychotics (such as haloperidol, clozapine, olanzapine, and quetiapine), immuno-suppressants (such as cyclosporin and methotrexate), neuroprotective agents, steroids (such as steroid hormones), corticosteroids (such as dexamethasone, predisone, and hydrocortisone), vitamins, minerals, nutraceuticals, anti-depressants (such as imipramine, fluoxetine, paroxetine, escitalopram, sertraline, venlafaxine, and duloxetine), anxiolytics (such as alprazolam and buspirone), anticonvulsants (such as phenyloin and gabapentin), vasodilators (such as prazosin and sildenafil), mood stabilizers (such as valproate and aripiprazole), anti-cancer drugs (such as anti-proliferatives), antihypertensive agents (such as atenolol, clonidine, amlopidine, verapamil, and olmesartan), laxatives, stool softeners, diuretics (such as furosemide), anti-spasmotics (such as dicyclomine), anti-dyskinetic agents, and anti-ulcer medications (such as esomeprazole).

Such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration in combination of a compound of the present invention with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second. Such sequential administration may be close in time or remote in time.

Another aspect of the present invention includes combination therapy comprising administering to the subject a therapeutically or prophylactically effective amount of a therapeutic agent according to the present invention and one or more other therapy including chemotherapy, radiation therapy, gene therapy, stem cell therapy, or immunotherapy.

Compounds A and B are alpha7-selective ligands. For example, Compounds A and B are alpha7 agonists with Ki values=1-2 nM in displacement studies using ³H-MLA in rat hippocampal tissues.

Compound A exhibits poor affinity for other nicotinic receptors, namely Ki>1000 nM, including alpha4beta2. Compound B is 2-(3-pyridinyl)-1-azabicyclo[3.2.2]nonane; it binds to alpha4beta2, but is not functionally agonistic at that receptor. In functional studies, Compounds A and B exhibited Eₘₐₓ values >50% in an electrophysiology functional assay in Xenopus laevis oocytes transiently expressing human alpha7 nicotinic receptors. The IC₅₀s for Compounds A and B are >10 micromolar at more than 60 targets in a receptor profile screen.

Compound C is nicotine, which exhibits dual pharmacology. It binds with high affinity to both alpha7 and alpha4beta2 nAChRs, based on displacement of [³H]-MLA and [³H]-nicotine binding, respectively.

Physiological Effects of Selective alpha7 nAChR Agonists

### Neurogenesis

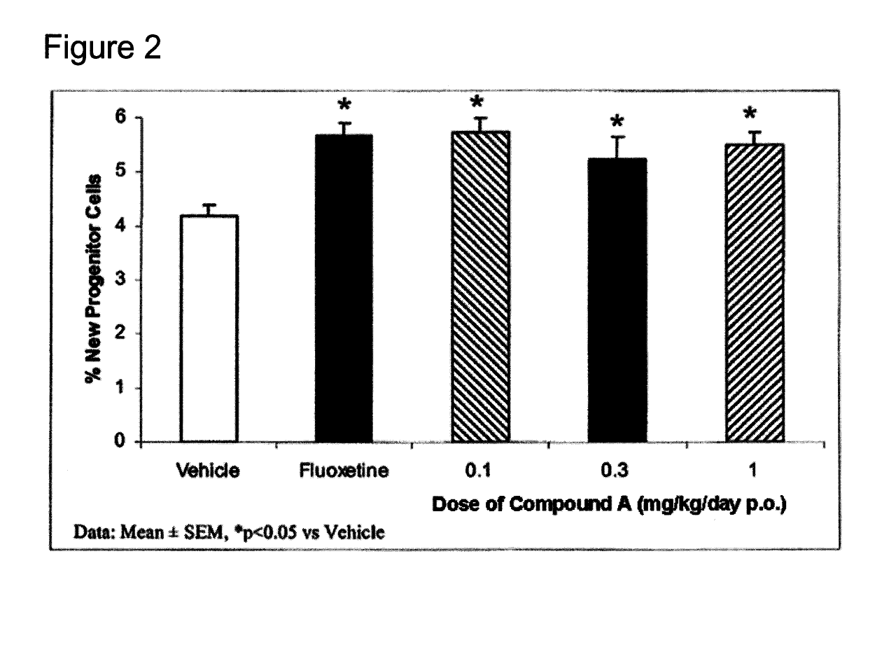

Neurogenesis in adult mammals occurs in specific brain regions, particularly in the subventricular and subgranular zones of the hippocampus. These newly generated granule cells, particularly those in the dentate gyrus of the hippocampus are believed to play a role in hippocampus-dependent learning and memory. Alterations in this process appear to be involved in the pathophysiology and treatment of mood and cognitive disorders. Using methods described by Shankaran et al. J Pharmacol Exp Ther 319: 1172-1182 (2006), hippocampal progenitor cell proliferation was assessed. Repeat administration of Compound A (0.1-1 mg/kg/day; p.o.) was shown to increase the proliferation of progenitor cells in the hippocampus of 129SvEv mice (FIG. 2).

As such, selective alpha7 compounds such as Compound A are believed useful in the treatment or prevention of disorders and conditions susceptible to amelioration through neurogenesis, namely through recruitment of neurogenesis including but not limited to learning and memory disorders, epilepsy, psychiatric disorders, including depression, bipolar disorder, and post traumatic stress disorder, and neurodegenerative diseases, including Alzheimer's disease, Parkinson's disease, amyotrophic lateral sclerosis, multiple sclerosis, frontotemporal dementia, Huntington's disease, and prion disease, as well as drug abuse or addiction and head trauma, such as stroke or physical injury, as well as the additional disorders and conditions herein described.

### Neuroinflammation

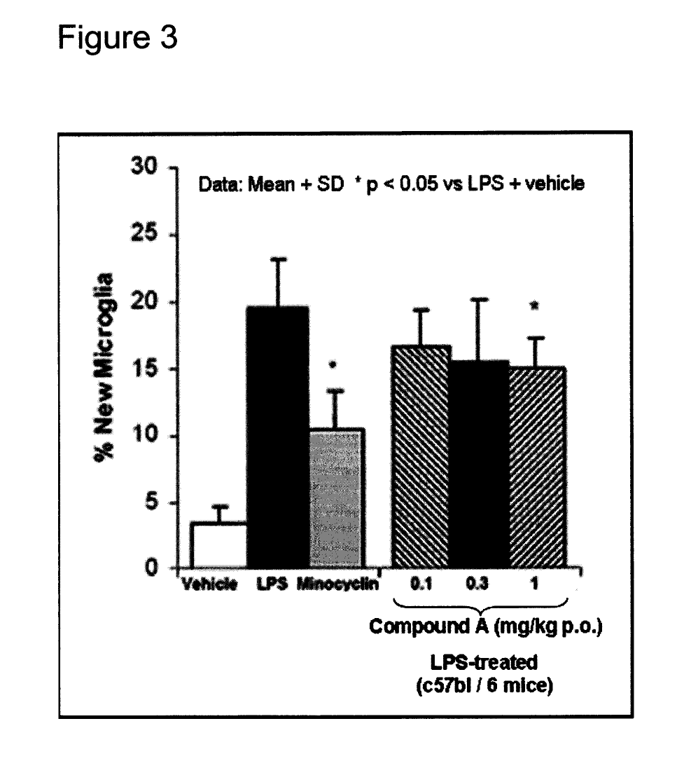

The proliferation of microglial cells is an early event in the activation process in animal models of neuroinflammation. Microglial activation is thought to contribute to the pathology of many CNS neuro-inflammatory and neurodegenerative disorders. It can be quantified by assessing the incorporation of deuterium from heavy water into the DNA of microglia (Shankaran et al., 2007). Compound A (1 mg/kg; p.o.) decreased LPS-induced neuroinflammation as measured by microglial proliferation in mice (FIG. 3).

As such, selective alpha7 compounds such as Compound A are believed useful in the treatment or prevention of the wide variety of CNS neuroinflammatory and neurodegenerative diseases, disorders, and conditions herein described.

Protection of Cells from Ionizing Radiation

The effects of the alpha7 nAChR-selective Compound A on ionizing radiation damage to rat brain vasculature endothelial cells (GP8.3 cell line) were studied. Cells were cultured in α-MEM/Ham's F10, 10% FBS, 50 IU/mL penicillin, 50 μg/mL streptomycin, 200 mM L-glutamine, and 250 μg/mL Geneticin and maintained in a humidified atmosphere containing 5% CO₂ at 37° C. Intracellular reactive oxygen species (ROS) generation was measured in GP8.3 cells using 2′7′ dichlorodihydrofluorescein diacetate (DCFH-DA). Cells were incubated with 20 μM DCFH-DA in phosphate buffered saline (PBS) for 30 min prior to irradiation. The fluorescence intensity was measured at excitation wavelength 485 nm and emission wavelength 530 nm using a Bio-Tek FL500 microplate fluorescence reader. Western blot analysis: GP8.3 cells were collected following treatment and protein was separated by polyacrylamide gel electrophoresis (PAGE) on a 12% gel. Primary goat anti-ICAM-1 and mouse anti-β-actin were used. The secondary antibody was horse radish peroxidase (HRP)-conjugated secondary antibodies. Northern blot analysis: Total RNA was isolated using Tri-ZOL reagent. cDNA probes were labeled with α32P-dCTP by the random primer extension method. The cDNA of rat ICAM-1 was synthesized. Cell viability was determined using a modified 3-(4,5-Dimethylthiazol-2-yl)-2,5-Diphenyltetrazolium Bromide (MTT) assay. Briefly, 5,000 cells/well were plated in 24-well plates and incubated overnight. Cells were then treated with 0-20 μg/mL either Compound A or mecamylamine for 72 h. At the end of the follow-up period, cells were incubated with MTT in PBS for 4 h. Cell lysis detergent (20% SDS and 50% dimethylformamide, pH 4.7, in PBS) was then added and the plates incubated overnight at 37° C. A 100 μl aliquot of the soluble fraction was then transferred to 96-well microplates, and the absorbance at 570 nm measured using an Enzyme-Linked ImmunoSorbent Assay (ELISA) plate reader.

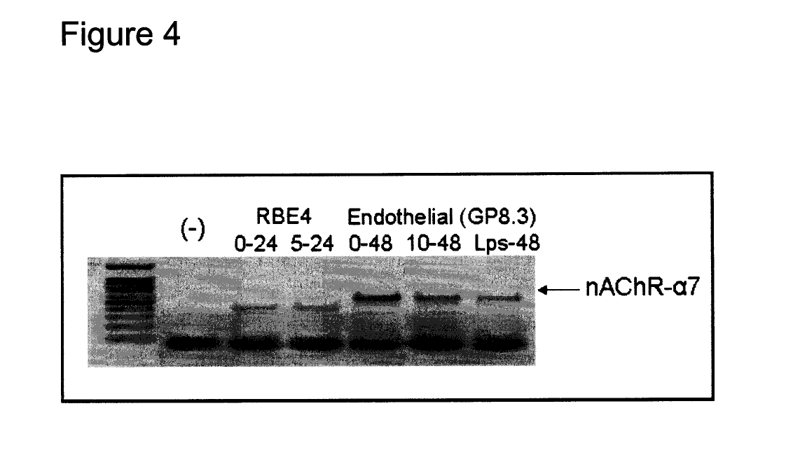

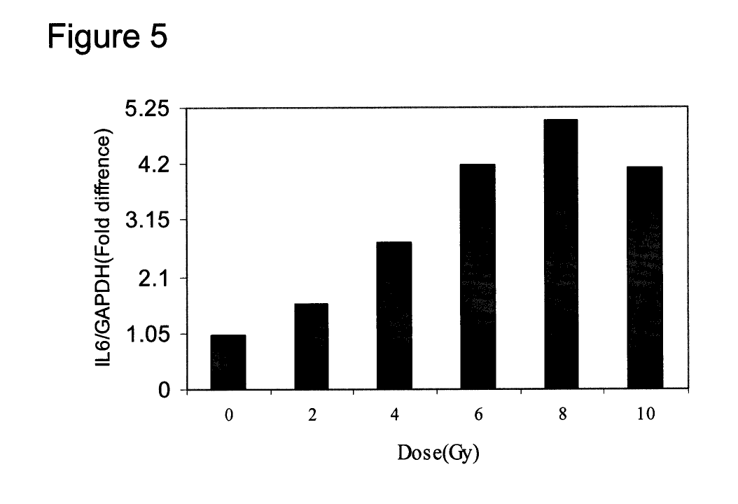

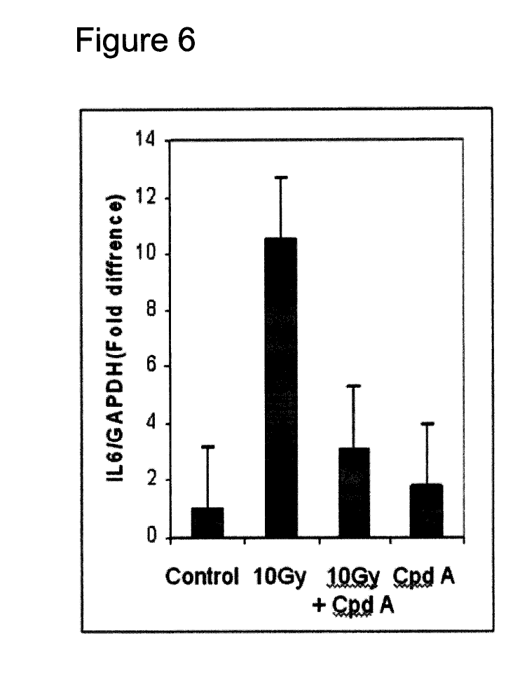

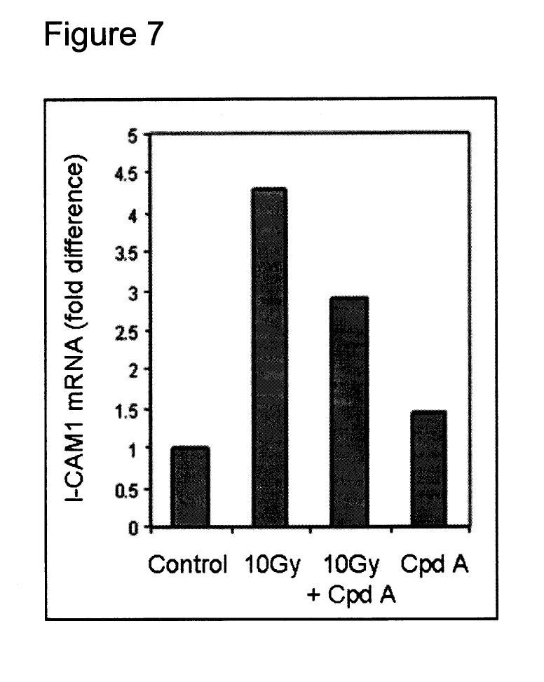

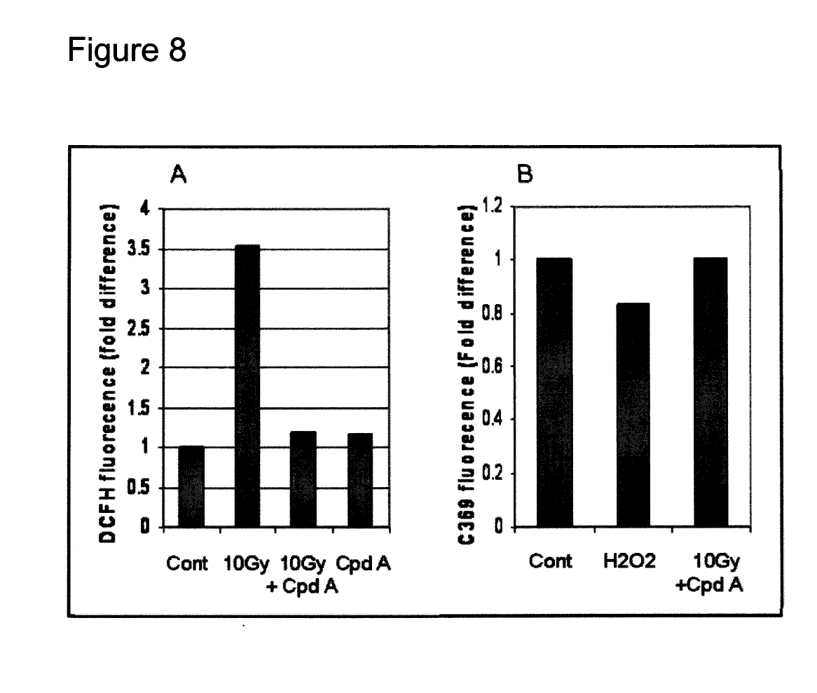

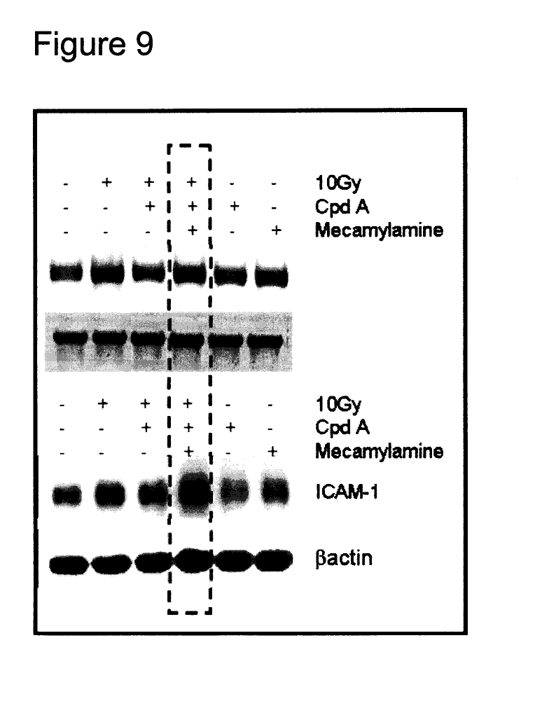

Reverse transcriptase polymerase chain reaction (RT-PCR) confirmed the presence of alpha7 nAChR subunits in the cultured endothelial cells (GP8.3) (FIG. 4). Ionizing radiation increased the expression of IL-6 and intercellular adhesion molecule-1 (ICAM-1) mRNA (protein) levels (FIGS. 5 and 9). Pre-incubating cells with 10 μM of the alpha7 ligand Compound A abolished the radiation-induced up-regulation of the pro-inflammatory cytokine IL-6 mRNA and protein (FIG. 6). Pre-incubation of cells with Compound A ameliorated radiation-induced up-regulation of 1-CAM1 mRNA and protein (FIG. 7). Pre-incubation of cells with Compound A also abolished radiation-induced up-regulation of ROS (FIG. 8). Finally, all of the aforementioned changes were reversed by an alpha7 nAChR antagonist mecamylamine, confirming that the effects were receptor-mediated (FIG. 9).

As such, a selective alpha7 agonist such as Compound A is believed to protect against radiation injury. These data suggest that a selective alpha7 antagonist may demonstrate the opposite effect and sensitize the cell lines to oxidative stress induced injury, thus providing alpha7 antagonists as a useful adjunct to directed radiotherapy.

Alternatively, an alpha7 antagonist may be applied locally, at the site of tumor excision, during or immediately following surgical ablation. Furthermore, since an alpha7 agonist is believed to protect against radiation injury, a combination therapy of an alpha7 antagonist locally to enhance the effectiveness of radiotherapy and an alpha7 systemically to protect healthy tissues before or during radiotherapy is believed to present a novel approach in the treatment and prevention of GBM.

### Protection of Stem Cell Implants

Cells in the hippocampus of the brain continue to proliferate and develop into mature neurons throughout the lifespan of animals and humans. Alterations of this process appear to be involved in the pathophysiology and the treatment of mood and cognitive disorders. Since new neurons develop from the progenitor cells in the hippocampus, measuring the proliferation of this cell population may be used as an indicator of the full neurogenesis process. Hippocampal neurogenesis can be evaluated by measuring the incorporation of deuterium from heavy water into the DNA of progenitor cells and the rate of label incorporation reflects the rate of cellular proliferation. Fluoxetine, a known neurogenic antidepressant, was used as the positive control and as a comparator to the nicotinic ligands.

Methods: 10 week old male 129SvEv mice (N=6 per group)
Duration of drug treatment: 3 weeks with Vehicle, Fluoxetine (10 mg/kg, po), or different doses of α7 compounds administered by oral gavage.
²H₂O labeling: Animals received a priming intraperitoneal bolus of 49 ml/kg>99% ²H₂O (Spectra Stable Isotopes, Columbia, Md.) containing 0.9% NaCl and were maintained on 10% ²H₂O in drinking water for the duration of the labeling period. Mice were labeled for the last 10-11 days of the drug treatment period.
Tissue processing and analysis: At the end of treatment and label, mice were sacrificed, the hippocampus was dissected from the brain and digested with papain and progenitor cells were isolated by Percoll fractionation. DNA was purified from the isolated progenitor cells using a DNEasy tissue kit (Qiagen, Valencia, Calif.), then processed and analyzed by GC/MS. DNA was enzymatically hydrolyzed to free deoxyribonucleosides, and the deoxyribose moiety of purine deoxyribonucleosides was converted to the pentafluorobenzyl triacetate derivative. GC/MS analysis was performed in negative chemical ionization mode using an Agilent (Palo Alto, Calif.) model 5973 mass spectrometer and a 6890 gas chromatograph fitted with a db-225 column. Selected ion monitoring was performed on ions with mass-to-charge ratios (m/z) 435 and 436, representing M₀ and M₁ mass isotopomers, respectively. Incorporation of ²H into purine deoxyribose was quantified as the molar excess fraction M₁ (EM₁), i.e. the increase over natural abundance (background), determined from the fractional M₁ value in unlabeled DNA standards from calf thymus.

$${EM}_{1} = \frac{{( abundance m \text{/} z 436 )}_{sample}}{{( abundance m \text{/} z 435 + 436 )}_{sample}} - \frac{{( abundance m \text{/} z 436 )}_{standard}}{{( abundance m \text{/} z 435 + 436 )}_{standard}}$$

Concurrent analysis of DNA from bone marrow cells, which are fully turned over in 1 week provides an internal reference factor in each animal to correct for variations in body water ²H enrichment. The fraction of newly divided progenitor cells was calculated as the ratio of EM₁ in purine deoxyribose from progenitor cell DNA to the corresponding EM₁ from enrichment in bone marrow DNA.

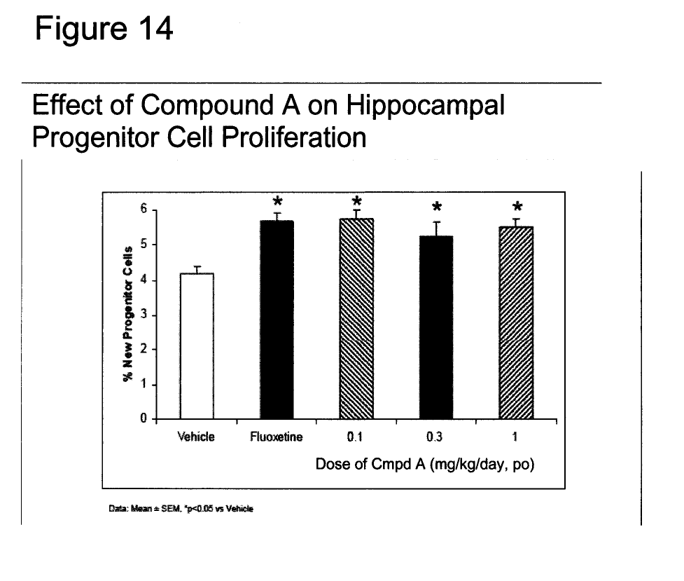

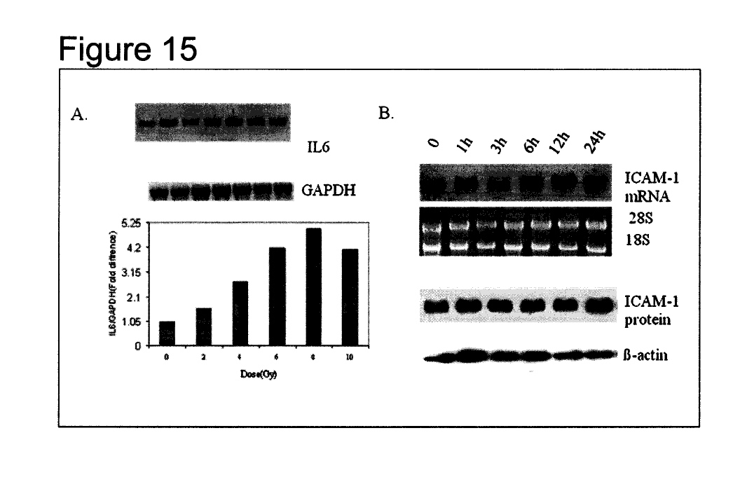

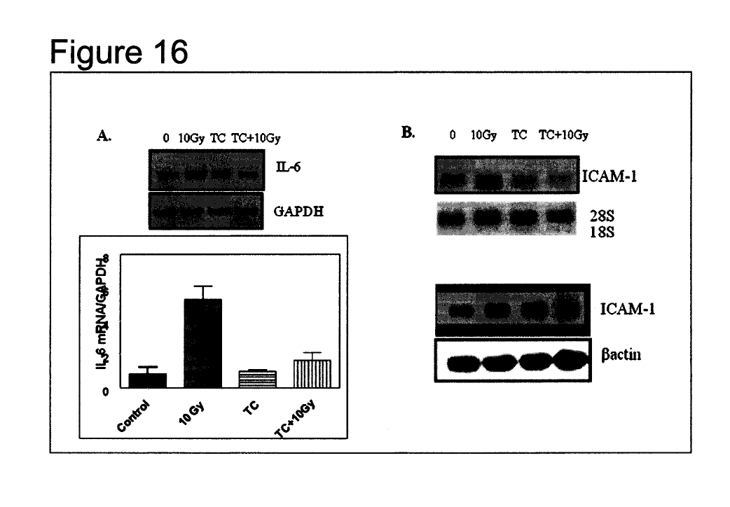

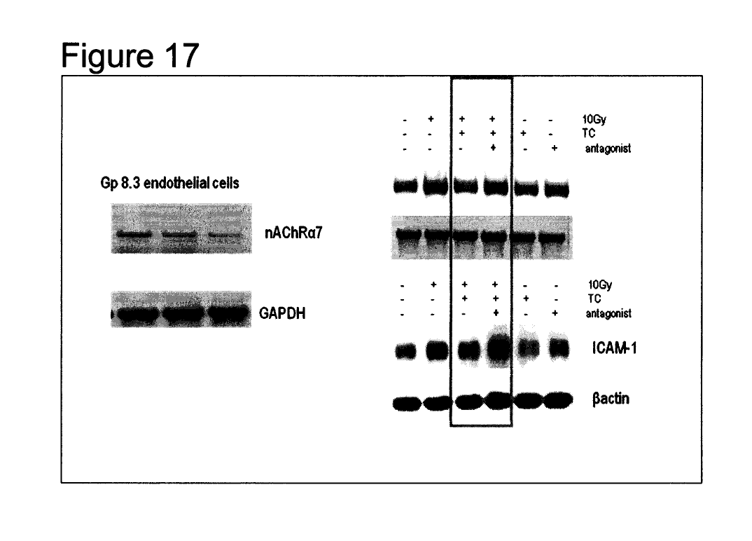

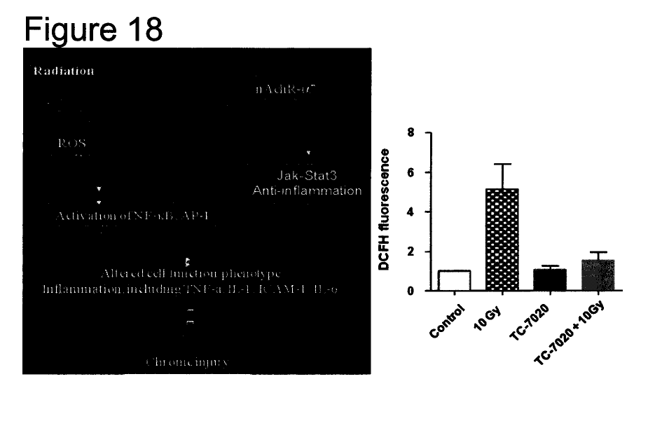

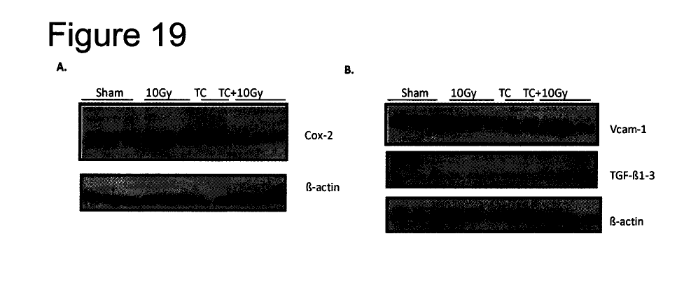

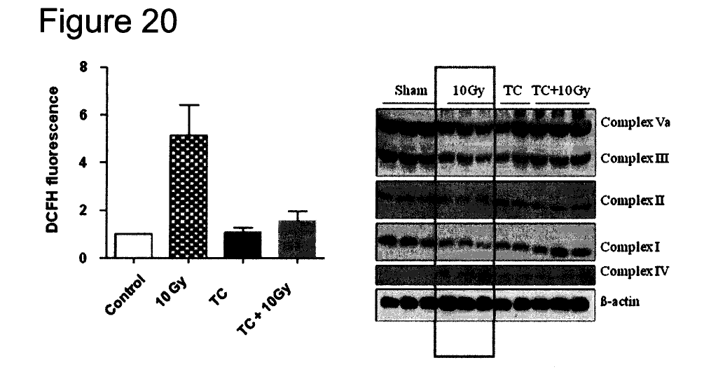

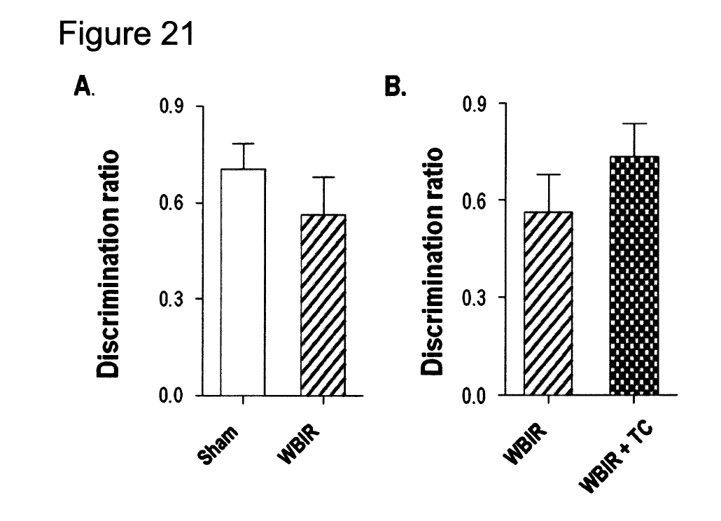

FIG. 14 demonstrates an effect of Compound A on hippocampal neurogenesis. Chronic treatment with Compound A produced an increase in the proliferation of hippocampal progenitor cells at all the doses tested in this study. One way ANOVA revealed a significant (p<0.05) difference among the treatment groups. Post-hoc comparison procedures (Holm-Sidak method) revealed a significant (*p<0.05) difference for the Compound A treatment groups compared to Vehicle. The magnitude of increase produced by 0.1, 0.3 and 1 mg/kg doses of Compound A was 36%, 24% and 30% respectively. The positive control fluoxetine also produced a 35% increase in the proliferation of hipocampal progenitor cells. Data represent mean±SEM of 15 mice per group. Compound A increased the proliferation of hippocampal progenitor cells in a dose-dependent manner, whereas an alpha4beta2-selective compound was without effect. As illustrated in FIGS. 15-21, these data demonstrate the neurogenic activity of nicotinic receptor ligands with potential therapeutic efficacy in mood and cognitive disorders.

### Neuroprotection

Several articles suggest a role for neuronal nicotinic acetylcholine receptors for neuroprotection. See. for example, Picciotto et al., Neuroprotection via nAChRs, Front BioSci., 2008 Jan. 1, 492-504; Quik et al., Nicotine Neuroprotection Against Nigrostriatal Damage, Trends Pharamcol Sci., 2007 May, 28(5), 229-35, Epub 2007 April; and O'Neill et al., The Role of Neuronal Nicotinic Acetylcholine Receptors in Acute and Chronic Neurodegeneration, Curr Drug Targets CNS Neurol Disord., 2002 August, 1(4), 399-411, each incorporated herein with regard to such teaching.

It has been shown that nicotine-induced complex formation between the alpha7 nicotinic acetylcholine receptor (nAChR) and the tyrosine-phosphorylated enzyme Janus kinase 2 (JAK2) results in subsequent activation of phosphatidylinositol-3-kinase (PI-3-K) and Akt. Nicotine interaction with the alpha7 nAChR inhibits Aβ (1-42) interaction with the same receptor, and Aβ (1-42)-induced apoptosis is prevented through nicotine-induced activation of JAK2. These effects can be shown by measuring markers of cytotoxicity, including the cleavage of the nuclear protein poly(ADP-ribose) polymerase (PARP), the induction of caspase 3, or cell viability.

PC12, rat pheochromocytoma cells, were maintained in proliferative growth phase in Dulbecco's modified Eagle's medium supplemented with 10% horse serum, 5% fetal calf serum, and antibiotics (penicillin/streptomycin). Apoptosis was determined by assessing the cleavage of the DNA-repairing enzyme PARP using a Western blot assay. PARP (116 kDa) is an endogenous substrate for caspase-3, which is cleaved to a typical 85-kDa fragment during various forms of apoptosis. PC12 cells were treated with 0.1 uM AR for 8 h in the presence or absence of Compound B and/or AG-490. The cells were collected, washed with PBS, and lysed in 1 ml of SDS-PAGE sample buffer boiled for 10 min. Total cell lysates (30 ug of protein) were separated by SDS-PAGE and transferred to nitrocellulose membranes. The membranes were blocked for 1 h at 25° C. with 5% nonfat dry milk in TBST (25 mM Tris-HCl, pH 7.5, 0.5 M NaCl, and 0.05% Tween 20). Membranes were incubated with primary PARP antibody specific for the 85-kDa fragments for 2 to 3 h at 25° C., rinsed with TBST, and incubated with secondary antibody for 1 h at 25° C. Immuno-detection was performed with appropriate antibody using an enhanced chemiluminescence system. Caspase 3 enzyme activity was determined with a fluorogenic substrate for caspase-3 in crude PC12 cell extracts. The caspase 3 fluorogenic peptide Ac-DEVD-AMC contains the specific caspase 3 cleavage sequence (DEVD) coupled at the C-terminal to the fluorochrome 7-amino-4-methyl coumarin. The substrate emits a blue fluorescence when excited at a wavelength of 360 nm. When cleaved from the peptide by the caspase 3 enzyme activity in the cell lysate, free 7-amino-4-methyl Coumarin is released and can be detected by its yellow/green emission at 460 nm. Appropriate controls included a reversible aldehyde inhibitor of caspase 3 to assess the specific contribution of the caspase 3 enzyme activity. Fluorescence units were normalized relative to total protein concentration of the cell extract.

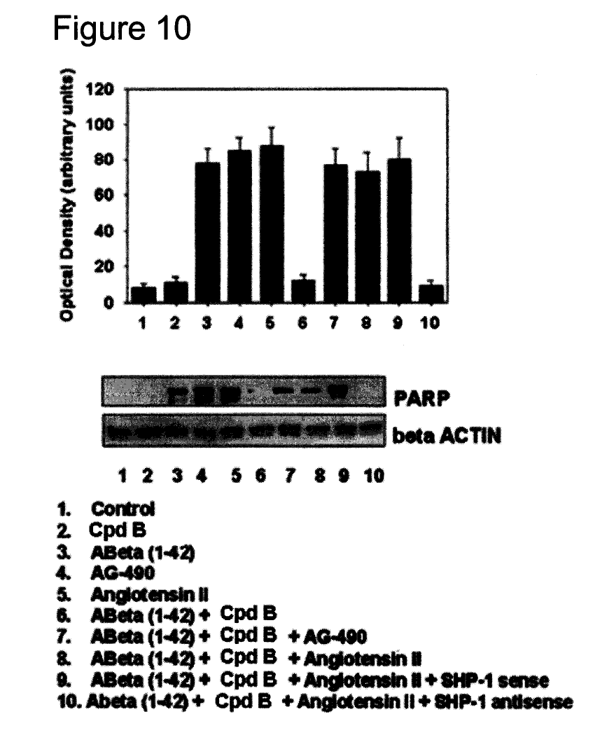

It was found that Compound B, a novel alpha7-selective agonist, exerts neuroprotective effects via activation of the JAK2/PI-3K cascade, which can be neutralized through activation of the angiotensin II (Ang II) AT2 receptor (FIG. 10). Vanadate not only augmented the Compound B-induced tyrosine phosphorylation of JAK2 but also blocked the Ang II neutralization of Compound B-induced neuroprotection against Aβ (1-42)-induced cleavage of PARP. Furthermore, when SHP-1 was neutralized via antisense transfection, the Ang II inhibition of Compound B-induced neuroprotection against Aβ (1-42) was prevented. These results support the hypothesis that JAK2 plays a central role in the nicotinic alpha7 receptor-induced activation of the JAK2-PI-3K cascade in PC12 cells, which ultimately contribute to nAChR-mediated neuroprotection.

Physiological Effects of Dual alpha4beta2/alpha7 Agonists

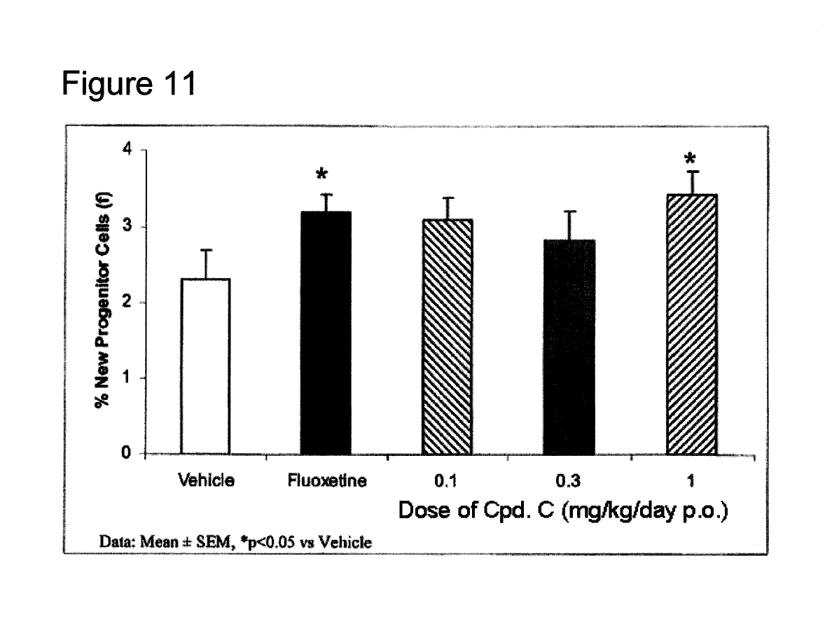

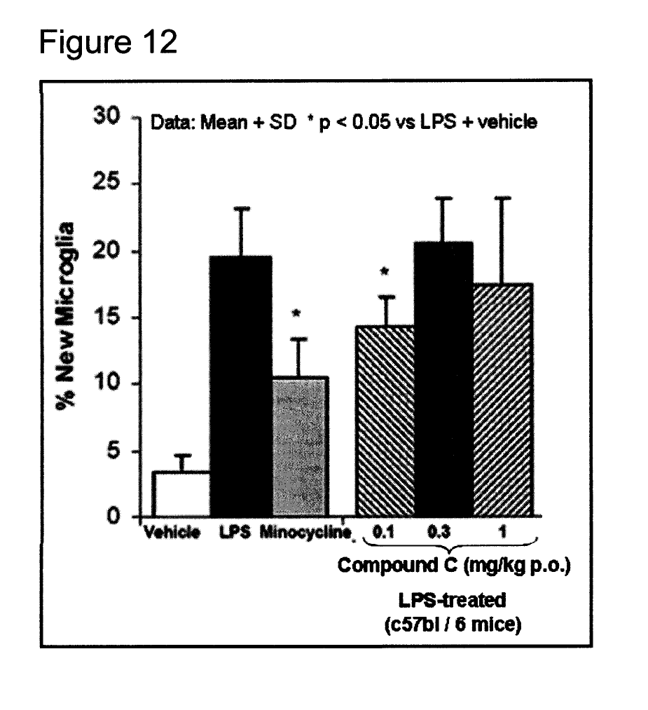

Repeat administration of the dual pharmacology, alpha4beta2/alpha7-selective Compound C (1 mg/kg/day; p.o.) was shown to increase the proliferation of progenitor cells in the hippocampus of 129SvEv mice (FIG. 11). Compound C (0.1 mg/kg; p.o.) also decreased LPS-induced neuroinflammation as measured by microglial proliferation in mice (FIG. 12).

As such, dual pharmacology compounds such as Compound C are believed to be useful in the treatment or prevention of the wide variety of CNS neuroinflammatory and neurodegenerative diseases, disorders, and conditions herein described. Dual pharmacology agonists are believed to minimize neuronal damage. Thus, a combination of an alpha4beta2 agonist and an alpha7 agonist, or a dual agonist, is believed useful in the prevention or treatment of “chemobrain” (chemotherapy-induced cognitive deficits), radiation-induced cognitive deficits, ischemic events, autoimmune CNS disorders, and a variety of other neurodegenerative disorders, especially those that involve neuro-inflammation.

These data provide, in addition, for a combination therapy of an alpha4beta2 antagonist, for correction of hypercholinergic tone, and an alpha7 agonist, for neurogenesis. This combination would be expected to address both the symptoms and the underlying cause of major depressive disorder and brain reward disorder indications. Thus, a combination of an alpha4beta2 antagonist and an alpha7 agonist, or a dual compound of similar pharmacology, is believed useful in the prevention or treatment of major depressive disorder, addictions, dysregulated food intake, bipolar disorder, and other similar disorders and conditions.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A method for treating or preventing disorders or conditions susceptible to recruitment of neurogenesis comprising administering a selective alpha7 agonist.

2. A method for providing neuroprotection comprising administering a selective alpha7 agonist.

3. (canceled)

4. (canceled)

5. The method of claim 1, wherein the alpha7 agonist increases the proliferation of progenitor cells in the hippocampus.

6. The method of claim 1, wherein the disorder or condition is selected from learning and memory disorders, epilepsy, psychiatric disorders, depression, bipolar disorder, post traumatic stress disorder, neurodegenerative diseases, Alzheimer's disease, Parkinson's disease, amyotrophic lateral sclerosis, multiple sclerosis, frontotemporal dementia, Huntington's disease, prion disease, substance abuse, addiction, dependency, head trauma, stroke, or physical injury.

7. The method of claim 1, wherein the disorder or condition is induced cognitive deficits and the selective alpha7agonist is administered in combination with an alpha4beta2 agonist.

8. The method of claim 7, wherein the administration is a single compound with dual alpha7 agonist and alpha4beta2 agonist pharmacology.

9. The method of claim 7, wherein the induced cognitive deficit is one or more of chemotherapy-induced cognitive deficit, radiation-induced cognitive deficit, ischemia-induced cognitive deficit, autoimmune and inflammatory disease induced cognitive deficit, inflammation-induced cognitive deficit, injury-induced cognitive deficit, and neuroinflammation.

10. The method of claim 1, wherein the disorder or condition is a neurobiological disorder selected from depression, major depressive disorder, addiction, physical dependence, psychological dependence, dysregulated food intake, or bipolar disorder and the selective alpha7 agonist is administered in combination with an alpha4beta2 antagonist.

11. The method of claim 10, wherein the administration is a single compound with dual alpha7 agonist and alpha4beta2 antagonist pharmacology.

12. The method of claim 1, wherein the disorder or condition is glioblastoma multiforme, and the selective alpha7 agonist is administered in combination with an alpha7 antagonist.

13. The method of claim 12, wherein the alpha7 agonist is administered systemically.

14. The method of claim 13, wherein the alpha7 antagonist is administered locally.

15. The method of claim 14, wherein the alpha7 antagonist is administered topically.

16. The method of claim 15, wherein the alpha7 antagonist is administered upon surgical ablation.

17. A method for protecting stem cells against host pathology implanted in a patient comprising administration of an alpha 7 agonist.

18. The method of claim 17, wherein the method of protecting includes the steps of:
implanting one or more stem cell; and
administering one or more alpha 7 agonist.

19. The method of claim 17, wherein the administration of the alpha7 agonist enhances survival and differentiation of stem cell implants.

20. The method of claim 17, wherein the administration of the alpha7 agonist induces hippocampal neurogenesis.

21. The method of claim 17, wherein the alpha7 agonist is a selective alpha7 agonist.

22. (canceled)

23. The method of claim 17, wherein the method treats one or more of stem-cell derived organ transplant, hematopoietic stem cell transplantation, bone marrow transplant, skin graft, cancer, neovascularization, angiogenesis, spinal cord injury, heart damage, haematopoiesis, baldness, deafness, blindness, vision impairment, birth defect, diabetes, orthopedics, and wound healing.

24. The method of claim 1, wherein the alpha7 agonist is administered adjunctively with one or more therapy.

25. The method of claim 24, wherein the therapy is a therapeutic agent.

26. The method of claim 24, wherein the therapy is radiation therapy, gene therapy, stem cell therapy, or immunotherapy.

27. The method of claim 24, wherein the alpha7 agonist is administered adjunctively with an SSRI for treating depression.

28. The method of claim 1, wherein the alpha7 agonist is a compound having a structure of Formula 1: 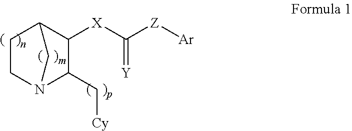
wherein
m is 2;
n is 1;
p is 1, 2, 3 or 4;
X is oxygen or NR′;
Y is oxygen or sulfur;
Z is NR′, a covalent bond or a linker species A;
A is selected from the group —CR′R″—, —CR′R″—CR′R″—, —CR′═CR′—, and —C═C—;
when Z is a covalent bond or A, X must be nitrogen;
Ar is an unsubstituted or substituted, carbocyclic or heterocyclic, monocyclic or fused polycyclic aryl group;
Cy is an unsubstituted or substituted 5- or 6-membered heteroaromatic ring; and substituents are selected from the group consisting of alkyl, alkenyl, heterocyclyl, cycloalkyl, aryl, substituted aryl, arylalkyl, substituted arylalkyl, halo, —OR′, —NR′R″, —CF₃, —CN, —NO₂, R′, —SR′, —N₃, —C(═O)NR′R″, —NR′C(═O)R″, —C(═O)R′, —C(═O)OR′, —OC(═O)R′, —O(CR′R″)ᵣC(═O)R′, —O(CR′R″)ᵣNR″C(═O)R′, —O(CR′R″)ᵣNR″SO₂R′, —OC(═O)NR′R″, —NR′C(═O)OR″, —SO₂R′, —SO₂NR′R″, and —NR′SO₂R″;
wherein each of R′ and R″ individually is hydrogen, C₁-C₈ alkyl, C₃₋₈ cycloalkyl, heterocyclyl, aryl, or arylalkyl; or
R′ and R″ can combine to form a 3 to 8 membered ring; and
r is 1, 2, 3, 4, 5, or 6,
or a pharmaceutically acceptable salt thereof.

29. (canceled)

30. (canceled)
